SEQUENCE LISTING (19 sequences)

SEQ ID NO: 1 - DNA, 1014 bp, Artificial Sequence
  Description of Artificial Sequence Synthetic construct
taatacgactcactataggggaattgtgagcggataacaattcccctctagaaataattttgtttaactttaagaaggagatataccatgggcagcagccatcatcatcatcatcacagcagcggcctggtgccgcgcggcagccatatggaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagtcgacaagcttgcggccgcactcgagcaccaccaccaccaccactgagat

SEQ ID NO: 2 - protein, 291 aa, Artificial Sequence
  Description of Artificial Sequence Synthetic construct
MGSSHHHHHHSSGLVPRGSHMETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 3 - DNA, 33 bp, Artificial Sequence
  Description of Artificial Sequence Synthetic primer
gctgacacatatggaaactgcagatctcaaatc

SEQ ID NO: 4 - DNA, 32 bp, Artificial Sequence
  Description of Artificial Sequence Synthetic primer
gtgactgtcgactcagtacatgtccctgtaga

SEQ ID NO: 5 - DNA, 1017 bp, Artificial Sequence
  Description of Artificial Sequence Synthetic construct
taatacgactcactataggggaattgtgagcggataacaattcccctctagaaataattttgtttaactttaagaaggagatataccatgggcagcagccatcatcatcatcatcacagcagcggcctggtgccgcgcggcagccatatggagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacatgtgcagctactgcaggtgatcaagaagacggagacagacatgagtcttcacccgctcctgcaggagatctacaaggacttgtactaggtcgacaagcttgcggccgcactcgagcaccaccaccaccaccactgagat

SEQ ID NO: 6 - protein, 292 aa, Artificial Sequence
  Description of Artificial Sequence Synthetic construct
MGSSHHHHHHSSGLVPRGSHMESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 7 - DNA, 33 bp, Artificial Sequence
  Description of Artificial Sequence Synthetic primer
gctcagacatatggagtccgctgacctccgggc

SEQ ID NO: 8 - DNA, 32 bp, Artificial Sequence
  Description of Artificial Sequence Synthetic primer
gtgactgtcgacctagtacaagtccttgtaga

SEQ ID NO: 9 - DNA, 1089 bp, Artificial Sequence
  Description of Artificial Sequence Synthetic construct
taatacgactcactataggggaattgtgagcggataacaattcccctctagaaataattttgtttaactttaagaaggagatataccatgaaaaaaggtcaccaccatcaccatcacggatcccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaagtcgaccaccaccaccaccaccactgagatccggctggccctactggccgaaaggaattcgaggccagcagggccaccgctgagcaataactagcataaccccttggggcctctaaacgggtcttgaggggttttttg

SEQ ID NO: 10 - protein, 287 aa, Artificial Sequence
  Description of Artificial Sequence Synthetic construct
MKKGHHHHHHGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 11 - DNA, 29 bp, Artificial Sequence
  Description of Artificial Sequence Synthetic primer
gttggatcccagtacaacccacaggtggc

SEQ ID NO: 12 - DNA, 32 bp, Artificial Sequence
  Description of Artificial Sequence Synthetic primer
gtgactgtcgacttagtacatgtccttgtaga

SEQ ID NO: 13 - protein, 25 aa, Artificial Sequence
  Description of Artificial Sequence Synthetic peptide
DGTPPPQEAEEPSLLKKLLLAPANT

SEQ ID NO: 14 - DNA, 10049 bp, Homo sapiens
gcgccgcctccttcggcgttcgccccacggaccggcaggcggcggaccgcggcccaggctgaagctcagggccctgtctgctctgtggactcaacagtttgtggcaagacaagctcagaactgagaagctgtcaccacagttctggaggctgggaagttcaagatcaaagtgccagcagattcagtgtcatgtgaggacgtgcttcctgcttcatagataagagtagcttggagctcggcggcacaaccagcaccatctggtcgcgatggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagttccttcagatcagccacaccttttccaggagttctgaagctgacagcactacaaaggagacgggggagcagcacgattttgcacaaatatccaccactttaaccttagagcttggacagtctgagctgtaggtaaccggcatattattccatatctttgttttaaccagtacttctaagagcatagaactcaaatgctgggggtaggtggctaatctcaggactgggaagattacggcgaattatgctcaatggtctgattttaactcacccgatgttaatcaatgcacattgctttagatcacattcgtgatttaccatttaattaactggtaacctcaaaattcgtggcctgtcttcccattcaccccgcttttgactattgtgctcctttataattctgaaaactaatcagcactttttaacaatgtttataatcctataagtctagatgtatccaaaggtgaagtatgtaaaaagcagcaaaatatttatttcaaagacttcacttctgtttcctgaatctaaagaaagacaacatgctgctttttaatcataggatggagaattttaaagaactgtttgggccaggcacagtcgctcatacttgtaatcccagcactttgggaggccgaggcgggtggatcacaaggtcagcagatcgagaccatcctggccaacatggtgaaaccctgtctctactaaaaatacaaaaattagccgggtgtggtggcacatgcctgtaatcccagctactcgggaagctgaggcaggagaattgcttgaaccagggagttggaggttgcagtgagctaagactgcaccactgcactccagcctggtgacagaacgagactctgtcttaaaaacaaacaaacaaaaaaaaaatctgttagataagctatcaaaatgcagctgttgttttgtttttggctcactgttttcgtggttgtaactaatatgtggaaaggcccatttccaggtttgcgtagaagagcccagaaaacagagtctcaagacccccgctctggactgtcataagctagcacccgtggtaagcgggacgagacaagctcccgaagcccgccagcttcctgctccactcagctccgtccagtcaacctgaacccacccagtccagctgtctgtgggaatggtggtgttcttagggacagactgacaccttacttgtcagtgttcctccgggccccatttggcagctcccgtatcttttgttatgttgcttttaaagatatgatgttttattgttttaactcttggtgacagtagatgctctctggagcgcagacgaggcacatgtgtcttcatagcctgggctgggtgggagccagtcaccctgcggatcgagagagggggtagagtcttcttcaaatggcagttttacttcaaatggcagatttcacaagagttggttattttttacaatggtttaggttgttaagtctcctttgtatgtaaggtagttttttcaacatctaaaatttttgttttagccttcaaaaccaacttaccaacctcagtccagctgggaaggcagcgttgattatggtagtttgtcaagaatatatggacctggaaacactttctctctctgtccacctggtagataaattgtcctgttgagaatttttagatctggactggaactgccaggaccaccgcctccagggagtcgctgggcacctggaggtatcgtcgatgcctctcccccatctttagaaaatttggctcttctgaggtcattattattttaagaatgattaggattgataagggtcccatgaccagcattatgaaaatgcgagagtgggaaggacacagtgtgagacttccactagaaaaaagtgaaagttagggttaggacatccttttttaaaaattacaaatttagtccgttttggtttttgtaatcaggctaggcacagtggctcacacatggaatcccagcactttgggaggccgaggtgggaggatcacttgagcccaggagttcgagaccagcctaggcaacatagcaagaccctgtctgtacacaaaatttaaaaattagttcatcggggtggcacacatcagtagtcccagctactctgcaggctgaggtgggaggattgcttgaacccaggaggtcgaggctgcagtgagctgtgatctcaccactgcattccagcctgggtgacagagttagattccaccctctcccaccccggcaaaaaaaaaaaaaaaagatgcaatcaaaggggctgttggccagcaatggcagcagcagcggcgggcagtctgcccaagtgtcttaggaaccaaaagcaaataaaagtgtttccatatatgccaccagccaagtggccatcctaattcagaaagaagctagcctttgagtgtctgtcatggtgcatccgtttcagtattatttcctaaaatgagaagcccctgtgtcaacaagatccaggggctggagcccaatgccaagcctgtgttgtccccagcgaccctgcagctgctcgctctgatgtaccctgtgccattcaaggagatgtggtccaggaaagtgagcctcatggttttcagagaagtcattgttctgtttacattttcataaaacctgtttaaaatagctccccgtctcaggctttcagcagtaacagtgagctgactggcaagttcgatgttagctcccgggacactcagcagcgatggtgagcattttggtttccttaaggcccagcaagacttccagggacatctctggtgaagccagaatggagacacccgtgacctcaggctgaaagtcactcgacattggtctcttgtgttgatagggaaggaaatcaggcattcctatttctttaaataacaaaaccactaattgccactcaatgctggaatattttgggtcacctaatcatagatttctcagggcatcaatactcaaatataggctgattatgccccagttcaaatgggaactattaacagagtgcatttcttgcttgctgggtttcaacagacatcagccaaaagaacaaaagagatgtcaggacagattccaggagtgtcggagcacatgtgtggcacccgctccctctggcagcgaatgtaggaagtcgccaaatttacccactcttcaacaagtcattgtttaaacacggtttttcattttctcaacttttaatagcaaaaagtgccaaagtcctcagagacctaacagccttggtctaccgtgctgaccagggtgaaggcacggcgagggactcctcccagacgtgcctcttgtgtgccagctggctgtggctcgggagcagacgcaggcctctccattgtccaggggagcctggcggcgcatccctcctctcccacctcctggcacttccagctgggtgtcccacatgttggattccgtccccaccacacttccagagaccggagaactgtgcagggcctaaggccgtttggatgaattgtcaaaacaagatgcttccagttacagcggcaggagcgggactgggagcacgggctgacggctgctggtgcctttcttcccacctcgcttgcctgtttccgcttgacccttcctccagctccgatgagaagagtataaagcatcttcctaacgggtgtgtttgctatacgaacataatggacgtgaagtggggcagaaacccagaactcagcattcaaggatgcccaggagagctgtccctgttttaaagagctgtgttttgttttgtttcgcatttagagagcagacaaggcacccttctgctgcgctgatacgtttcttacactgggccattttagacccccagggaaacagccttcctggagcgttgtctggaggttccagggacagggcagcctcccagagccgagcaagagctcaaggtacaaatgagagatttgctataccgtgagaagtcaacaacttagccaccacttccccgcaatggaccatgtaacaaatacctcagcaggccctgcaaaaggccatgctagagctgaggcgcacagcctgtggcctctgtagttagggcaggtgggatggagactccttgagtgcacacacctgagcctgcccacacacaggggagcagcatctcgtatgacgtctggaaggaacttcggttgtgtaaagggagccttgaagatacgtgcaaaaggtgctaccccaatttggtgaaactgacattgggcacgtcttgggcttaggagaagcggccgatggtcccggcctgcagtgacaaacccccctccccgcaccgcccccagcaccccctctcctcttcacctcttcctgctggccacgaggaagccacttcctcagagagaccctaccagatgcggatggaaacagatgcaccaaagcaagccctgatgaaaccgcgacttcctaaggtctgtctcctctgaacttgcacctgggcctctctgtgtttggttccaagcacttcccacctcaaactcccattttcaaaccactgtatctctgcgcacatctgctacttaccagccgcatacatgatggagggttttttggtcctgatccagtggccacacctgtctttgaaatgtctcactgaactccagttttaaaatagattcattgcttcaacacagcaagcccaatgcacccagctaagactggcttgaccgacagcctggcctttggtggggggcttcctggggcctggggaaagctggccaccttcaacagctggtacctcttcaacagtgtggcctttcaaaatgcagatgccaccaggagaacatgcccacagctcaccacctatggatgccatggctctgggcagctttcaaagcaggttcctgtggtctcctcagctgtttgagggggtaacagcaaatcagcctccattttaaaatgaaaacaccagcctccagatgtagggcctgctgggtgttgctagccgctggtccccaggcacggtgcactttctccacctcctgcagcctccctgttgtttctagactcttgcacctggtgagtgcaaggataggtgacccaggggcctgcagccttgtcctcagctcccatctcctggactgccagcctcaccctctgcagttagcatggttggcctgatgcagggatcccgagggattactttttagaccttctttcacattcagaaaagtagtatagattcaggagaggcaagaaaattatgctgtccatagaagtcacccatgaagactgatgccaccacctgaaggctcatgattgttaaaaatgtccacgggaacctctcgtccacaggaggtttgtctcaacacttcccatttttacggcattggcattgccaagcatggggaagtatctgctcttctcatgttaaaagtggcccagcttttcttaactcagtccaagctgacttgtttagctgcactggaatttcttaccaaccaaatatttgcatcgagcaaagggggctgtgtgcacctccctaatggcagcgatgatggctgctgtcattcaagcccatcttcagacgtcacagtctggaagtgaaatgtccacaaacatctgtggcagaaaaggctatacggaccacccagttgtgctgcagctttacagagcaaggaagggttgtggcaaataaatgattaacctgcctcgactgtgctgagggcaacaaaggccatctcaccaaaggattattcgatgccattaaatcatcccgtgaccttcctgcttccgagtccatggcctttgcccagggcatgtactcccctgagaggccttctgcctagaaagatctatgactgggttccaaagttgaggcctaggtttttgctgggatttagatattttcaggcaccattttgacagcattcaggaaaacggttattgaccccatagactagggtaagaataaaggcaataaatttggtctgactcagaatataggagatccatatatttctctggaaaccacagtgtacactaaaatgtgaaattgaaggttttgttaaaaagaaaaagataatgagcttcatgctttgtttaattacataatgatttccattacgctatttctgtgaaatgcagcaggttcttaaacgttatttcagtggcatgggctggaagcttatcacaaaaagccatgtgtgtggccttatcagaacagaaagagacaggctggtgcccaaggctgctgcctgctccaccttttgccagctctggacatctgaggacgtcccggcagatctggaatggggccctcaactgaccatttgcttctcagaatttcagtttgagacatgagaggtataatcagttacttttctccccccagagaaacccttttgtgaggggagaggagctatggtatgtggttcagctgaaacacatacaactgcatccttttggagtcctttgccaacaaaaacagaccaacagaccagatggtgtccatgttcaatatcatgtcttgatggacgcagctgatgacctcaaatacttgagtggtctcatggctgttagatggattatttgaaaaaaaaaaaaaaaaaagagagaaaaaataattgatttttacatcagagatagcaaactaagacctggggaggggggtcagcttttattttattttattttttttaagtttgctagttgggtcaaatgtgaggaggagggagtctacctgccacctcttctcttgcccctcttctgcccacacatccagcatccaaaatccattcatttaatgaattgataaagtgccgtgcaaactggtgcacaaacaggcccccagtccacgcagcctggctcctaggaaaagtggtgaccgggcgtgggggggcatgccgcagccctgggacacagtcgggcaccttccccggacccccaggccttggctgtgcctcaagtcagagagggtcagccttcaggccccggagacgagtgactggccgatcatttcacaataaaatcactcacttttggcaacttcactttttttaaggcacagtcagttccttttctcatgtacctcacaaaagatgaagaccatgtagtactctttttggtaaagttacagtgttcatgttaaatatcacttttttctacattgtgtggtaaaaagaactacgttaatagctatatcttaaatactgtgatttgactttttgaaaaatatcctaatacaaatattttactaacttacaatcactcatttaataagaaacatttggattcttttgaaatcagtgttaattgactcatattcttaaaagcctggctcttgaccctattggaaacacaaaggaagctgaaatcaaacatctaaaatacactgcgtacacgtgtgcgtgcacacacacacacacacacacacacacacagctcttcatttctcctgagccatgcagaatttactttcaatgtggaaatctgttccctttaccacactgtatatgcacagagcacaagagaggctatctctagtcacttccaccagcgaggccttagactccgtattagaggccaccgatttcatacaacagtgtttcgctaaagacccttcactattcttgtttagtaaatagctgtctgctcttcagggaactgttacctatgggttattaccaaagaacgctggcaattggaaatgtcctgatggaaattctttgcacgtgccggttctctggcatcctccaggtggcccaacccaaagcagaaagcagaaaccacagaccccgtgagtctccccataccttgtttccaataacttggcaaaacttcttggtgcatattggttacaccctctgggattcataatgccattaggctaaaaccctaagagagagggttgacagaaacacacgcgagaatgaggcagatcccagagcaaggactgggcccagactctccacatgtgctctactagtgagtgccttatactctcagtattttggggcttacagcttcttatttgtgctaaaaaggtgcagttccaaagtaggaactgccacacaggccccagcatcctctctccaacttcatacctctctcctggtggggggagcgggcatccaggacctccggaatcaaggatgtgcagagaagagcgaaagtaatttttctagtcacatgaactgattggttccaggcaattagaaaatggctataaaataaccttaattttaaaaaaaaatcttgggtcttcgttttcctattaggagactgaactgaccacatgtattgatttatatcctgaatatatgggaacttctgtgtttgggatgtcctactgtaagactgatgaatgtacagagttaatttcagggtacagttttgccttaatggttttaaaaaataaactattttttaaaatttt

SEQ ID NO: 15 - protein, 468 aa, Homo sapiens
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 16 - DNA, 1863 bp, Homo sapiens
actgatgtcttgactcatgggtgtattcacaaattctgttacttcaagtctttttcttttaacggattgatcttttgctagatagagacaaaatatcagtgtgaattacagcaaacccctattccatgctgttatgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaaaaaaaaaaaaaaa

SEQ ID NO: 17 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 18 - DNA, 3734 bp, Homo sapiens
gcggagcgtgtgacgctgcggccgccgcggacctggggattaatgggaaaagttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagcccttgagcacccggcctggagcagcagagtcccacgatcgccctcagacacatgacacccacggcctctggctccctgtgccctctctcccgcttcctccagccagctctcttcctgtctttgttgtctccctctttctcagttcctctttcttttctaattcctgttgctctgtttcttcctttctgtaggtttctctcttcccttctcccttgccctccctttctctctccaccccccacgtctgtcctcctttcttattctgtgagatgttttgtattatttcaccagcagcatagaacaggacctctgcttttgcacaccttttccccaggagcagaagagagtggggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtgggcaggaggccccaggctgagagccagatgcctccccaagactgtcattgcccctccgatgctgaggccacccactgacccaactgatcctgctccagcagcacacctcagccccactgacacccagtgtccttccatcttcacactggtttgccaggccaatgttgctgatggccccctgcactggccgctggacggcactctcccagcttggaagtaggcagggttccctccaggtgggcccccacctcactgaagaggagcaagtctcaagagaaggaggggggattggtggttggaggaagcagcacacccaattctgcccctaggactcggggtctgagtcctggggtcaggccagggagagctcggggcaggccttccgccagcactcccactgcccccctgcccagtagcagccgcccacattgtgtcagcatccagggccagggcctggcctcacatccccctgctcctttctctagctggctccacgggagttcaggccccactccccctgaagctgcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaa

SEQ ID NO: 19 - protein, 441 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

(19) United States
(12) Patent Application Publication
(10) Pub. No.: US 2008/0234349 A1
(43) Pub. Date: Sep. 25, 2008

(54) PPAR active compounds

(76) Inventors: Jack Lin, Hercules, CA (US); Shenghua Shi, San Diego, CA (US); Chao Zhang, Moraga, CA (US); Rebecca Zuckerman, Alameda, CA (US); Dean R. Artis, Kensington, CA (US); Prabha N. Ibrahim, Mountain View, CA (US); Byunghun Lee, Marina, CA (US)

Correspondence Address: FOLEY & LARDNER LLP, P.O. BOX 80278, SAN DIEGO, CA, 92138-0278, US

(21) Appl. No.: 11/517,010

(22) Filed: Sep. 6, 2006

Related U.S. Application Data
(60) Provisional application No. 60/715,259, filed on Sep. 7, 2005.

(51) Int. Cl. A61K 31/4155; C07D 409/12; A61K 31/4025; A61P 25/00; A61P 3/04; C07D 409/14

(52) U.S. Cl. 514/406; 548/465; 514/414; 548/364.7

(57) ABSTRACT

Compounds are described that are active on at least one of PPARα, PPARδ, and PPARγ, which are useful for therapeutic and/or prophylactic methods involving modulation of at least one of PPARα, PPARδ, and PPARγ.

### RELATED PATENT APPLICATIONS

This application claims the benefit of U.S. Prov. App. No. 60/715,259, filed Sep. 7, 2005, which is incorporated herein by reference in its entirety and for all purposes.

### FIELD OF THE INVENTION

The present invention relates to the field of modulators for members of the family of nuclear receptors identified as peroxisome proliferator-activated receptors.

### BACKGROUND OF THE INVENTION

The following description is provided solely to assist the understanding of the reader. None of the references cited or information provided is admitted to be prior art to the present invention. Each of the references cited herein is incorporated by reference in its entirety, to the same extent as if each reference were individually indicated to be incorporated by reference herein in its entirety.

The peroxisome proliferator-activated receptors (PPARs) form a subfamily in the nuclear receptor superfamily. Three isoforms, encoded by separate genes, have been identified thus far: PPARγ, PPARα, and PPARδ.

There are two PPARγ isoforms expressed at the protein level in mouse and human, γ1 and γ2. They differ only in that the latter has 30 additional amino acids at its N terminus due to differential promoter usage within the same gene, and subsequent alternative RNA processing. PPARγ2 is expressed primarily in adipose tissue, while PPARγ1 is expressed in a broad range of tissues.

Murine PPARα was the first member of this nuclear receptor subclass to be cloned; it has since been cloned from humans. PPARα is expressed in numerous metabolically active tissues, including liver, kidney, heart, skeletal muscle, and brown fat. It is also present in monocytes, vascular endothelium, and vascular smooth muscle cells. Activation of PPARα induces hepatic peroxisome proliferation, hepatomegaly, and hepatocarcinogenesis in rodents. These toxic effects are not observed in humans, although the same compounds activate PPARα across species.

Human PPARδ was cloned in the early 1990s and subsequently cloned from rodents. PPARδ is expressed in a wide range of tissues and cells with the highest levels of expression found in the digestive tract, heart, kidney, liver, adipose, and brain.

The PPARs are ligand-dependent transcription factors that regulate target gene expression by binding to specific peroxisome proliferator response elements (PPREs) in enhancer sites of regulated genes. PPARs possess a modular structure composed of functional domains that include a DNA binding domain (DBD) and a ligand binding domain (LBD). The DBD specifically binds PPREs in the regulatory region of PPAR-responsive genes. The DBD, located in the C-terminal half of the receptor contains the ligand-dependent activation domain, AF-2. Each receptor binds to its PPRE as a heterodimer with a retinoid X receptor (RXR). Upon binding an agonist, the conformation of a PPAR is altered and stabilized such that a binding cleft, made up in part of the AF-2 domain, is created and recruitment of transcriptional coactivators occurs. Coactivators augment the ability of nuclear receptors to initiate the transcription process. The result of the agonist-induced PPAR-coactivator interaction at the PPRE is an increase in gene transcription. Downregulation of gene expression by PPARs appears to occur through indirect mechanisms. (Bergen & Wagner, 2002, Diabetes Tech. & Ther., 4:163-174).

The first cloning of a PPAR (PPARα) occurred in the course of the search for the molecular target of rodent hepatic peroxisome proliferating agents. Since then, numerous fatty acids and their derivatives, including a variety of eicosanoids and prostaglandins, have been shown to serve as ligands of the PPARs. Thus, these receptors may play a central role in the sensing of nutrient levels and in the modulation of their metabolism. In addition, PPARs are the primary targets of selected classes of synthetic compounds that have been used in the successful treatment of diabetes and dyslipidemia. As such, an understanding of the molecular and physiological characteristics of these receptors has become extremely important to the development and utilization of drugs used to treat metabolic disorders.

Kota et al., 2005, Pharmacological Research 51: 85-94, provides a review of biological mechanisms involving PPARs that includes a discussion of the possibility of using PPAR modulators for treating a variety of conditions, including chronic inflammatory disorders such as atherosclerosis, arthritis and inflammatory bowel syndrome, retinal disorders associated with angiogenesis, increased fertility, and neurodegenerative diseases.

Yousef et al., 2004, Journal of Biomedicine and Biotechnology 2004(3):156-166, discusses the anti-inflammatory effects of PPARα, PPARγ and PPARδ agonists, suggesting that PPAR agonists may have a role in treating neuronal diseases such as Alzheimer's disease, and autoimmune diseases such as inflammatory bowel disease and multiple sclerosis. A potential role for PPAR agonists in the treatment of Alzheimer's disease has been described in Combs et al., 2000, Journal of Neuroscience 20(2): 558, and such a role for PPAR agonists in Parkinson's disease is discussed in Breidert et al. Journal of Neurochemistry, 2002, 82: 615. A potential related function of PPAR agonists in treatment of Alzheimer's disease, that of regulation of the APP-processing enzyme BACE, has been discussed in Sastre et al. Journal of Neuroscience, 2003, 23(30):9796. These studies collectively indicate PPAR agonists may provide advantages in treating a variety of neurodegenerative diseases by acting through complementary mechanisms.

Discussion of the anti-inflammatory effects of PPAR agonists is also available in Feinstein, Drug Discovery Today: Therapeutic Strategies , 2004, 1(1):29-34 in relation to multiple sclerosis and Alzheimer's disease; Patel et al., The Journal of Immunology, 2003, 170:2663-2669 in relation to chronic obstructive pulmonary disease (COPD) and asthma; Lovett-Racke et al., The Journal of Immunology, 2004, 172:5790-5798 in relation to autoimmune disease; Malhotra et al., Expert Opinions in Pharmacotherapy, 2005, 6(9):1455-1461 in relation to psoriasis; and Storer et al., Journal of Neuroimmunology, 2005, 161:113-122 in relation to multiple sclerosis.

This wide range of roles for the PPARs that have been discovered suggest that PPARα, PPARγ and PPARδ may play a role in a wide range of events involving the vasculature, including atherosclerotic plaque formation and stability, thrombosis, vascular tone, angiogenesis, cancer, pregnancy, pulmonary disease, autoimmune disease, and neurological disorders.

Among the synthetic ligands identified for PPARs are thiazolidinediones (TZDs). These compounds were originally developed on the basis of their insulin-sensitizing effects in animal pharmacology studies. Subsequently, it was found that TZDs induced adipocyte differentiation and increased expression of adipocyte genes, including the adipocyte fatty acid-binding protein aP2. Independently, it was discovered that PPARγ interacted with a regulatory element of the aP2 gene that controlled its adipocyte-specific expression. On the basis of these seminal observations, experiments were performed that determined that TZDs were PPARγ ligands and agonists and demonstrate a definite correlation between their in vitro PPARγ activities and their in vivo insulin-sensitizing actions. (Bergen & Wagner, supra).

Several TZDs, including troglitazone, rosiglitazone, and pioglitazone, have insulin-sensitizing and anti-diabetic activity in humans with type 2 diabetes and impaired glucose tolerance. Farglitazar is a very potent non-TZD PPAR-γ-selective agonist that was recently shown to have antidiabetic as well as lipid-altering efficacy in humans. In addition to these potent PPARγ ligands, a subset of the non-steroidal antiinflammatory drugs (NSAIDs), including indomethacin, fenoprofen, and ibuprofen, have displayed weak PPARγ and PPARα activities. (Bergen & Wagner, supra).

The fibrates, amphipathic carboxylic acids that have been proven useful in the treatment of hypertriglyceridemia, are PPARα ligands. The prototypical member of this compound class, clofibrate, was developed prior to the identification of PPARs, using in vivo assays in rodents to assess lipid-lowering efficacy. (Bergen & Wagner, supra).

Fu et al., Nature, 2003, 425:9093, demonstrated that the PPARα binding compound, oleylethanolamide, produces satiety and reduces body weight gain in mice.

Clofibrate and fenofibrate have been shown to activate PPARα with a 10-fold selectivity over PPARγ. Bezafibrate acts as a pan-agonist that shows similar potency on all three PPAR isoforms. Wy-14643, the 2-arylthioacetic acid analogue of clofibrate, is a potent murine PPARα agonist as well as a weak PPARγ agonist. In humans, all of the fibrates must be used at high doses (200-1,200 mg/day) to achieve efficacious lipid-lowering activity.

TZDs and non-TZDs have also been identified that are dual PPARγ/α agonists. By virtue of the additional PPARα agonist activity, this class of compounds has potent lipid-altering efficacy in addition to antihyperglycemic activity in animal models of diabetes and lipid disorders. KRP-297 is an example of a TZD dual PPARγ/α agonist (Fajas, 1997, J. Biol. Chem., 272:18779-18789); furthermore DRF-2725 and AZ-242 are non-TZD dual PPARγ/α agonists. (Lohray, et al., 2001, J. Med. Chem., 44:2675-2678; Cronet, et al., 2001, Structure (Camb.) 9:699-706).

In order to define the physiological role of PPARδ, efforts have been made to develop novel compounds that activate this receptor in a selective manner. Amongst the α-substituted carboxylic acids previously described, the potent PPARδ ligand L-165041 demonstrated approximately 30-fold agonist selectivity for this receptor over PPARγ, and it was inactive on murine PPARα (Liebowitz, et al., 2000, FEBS Lett., 473:333-336). This compound was found to increase high-density lipoprotein levels in rodents. It was also reported that GW501516 was a potent, highly-selective PPARδ agonist that produced beneficial changes in serum lipid parameters in obese, insulin-resistant rhesus monkeys. (Oliver et al., 2001, Proc. Natl. Acad. Sci., 98:5306-5311).

In addition to the compounds discussed above, certain thiazole derivatives active on PPARs have been described. (Cadilla et al., Internat. Appl. PCT/US01/149320, Internat. Publ. WO 02/062774, incorporated herein by reference in its entirety.)

Some tricyclic-α-alkyloxyphenylpropionic acids have been described as dual PPARα/γ agonistsin Sauerberg et al., 2002, J. Med. Chem. 45:789-804.

A group of compounds that are stated to have equal activity on PPARα/γ/δ is described in Morgensen et al., 2002, Bioorg. & Med. Chem. Lett. 13:257-260.

Oliver et al., describes a selective PPARδ agonist that promotes reverse cholesterol transport. (Oliver et al., supra)

Yamamoto et al., U.S. Pat. No. 3,489,767 describes “-(phenylsulfonyl)-indolyl aliphatic acid derivatives” that are stated to have “antiphlogistic, analgesic and antipyretic actions.” (Col. 1, lines 16-19.)

Kato et al., European patent application 94101551.3, Publication No. 0 610 793 A1, describes the use of 3-(5-methoxy-1-p-toluenesulfonylindol-3-yl)propionic acid (page 6) and 1-(2,3,6-triisopropylphenylsulfonyl)-indole-3-propionic acid (page 9) as intermediates in the synthesis of particular tetracyclic morpholine derivatives useful as analgesics.

Accordingly, there is a need for safer, more effective PPAR agonists for the treatment of a variety of diseases, including PPARα, PPARγ or PPARδ selective agonists as well as agonists selective for any two or all three of PPARα, PPARγ and PPARδ.

### SUMMARY OF THE INVENTION

The present invention relates to compounds active on PPARs, which are useful for therapeutic and/or prophylactic methods involving modulation of at least one of PPARα, PPARδ, and PPARγ. Included are compounds that have significant pan-activity across the PPAR family (PPARα, PPARδ, and PPARγ), as well as compounds that have significant specificity (at least 5-, 10-, 20-, 50-, or 100-fold greater activity) on a single PPAR, or on two of the three PPARs.

In one aspect, the invention includes compounds of Formula I as follows:

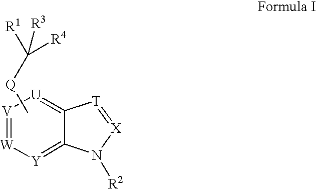

all salts, prodrugs, tautomers and isomers thereof,
wherein:
T, W, X, and Y are selected from N or CR⁵, one of U and V is C bound to Q and the other is either N or CR⁵, wherein at most two of W, Y and U or V is N and at most one of T and X is N;
Q is —O—, —S—, —NR⁶—, or —CR⁷R⁸—;
R¹ is selected from the group consisting of —C(O)OR¹⁷, —C(O)NR¹⁸R¹⁹, and a carboxylic acid isostere;
R² is selected from the group consisting of optionally substituted lower alkenyl, optionally substituted lower alkynyl, optionally substituted cycloalkyl, optionally substituted heterocycloalkyl, optionally substituted aryl, optionally substituted heteroaryl, —C(Z)NR⁹R¹⁰, —C(Z)R¹¹, —S(O)₂NR⁹R¹⁰, and —S(O)₂R¹²;
R³, R⁴, R⁷ and R⁸ are independently selected from the group consisting of hydrogen, optionally substituted lower alkyl, optionally substituted cycloalkyl, optionally substituted heterocycloalkyl, optionally substituted aryl, and optionally substituted heteroaryl; or
any two of R³, R⁴, R⁷ and R⁸ may combine to form optionally substituted 3-7 membered monocyclic cycloalkyl or optionally substituted 3-7 membered monocyclic heterocycloalkyl;
R⁵ at each occurrence is independently selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OR¹³, —SR¹⁴, —NR¹⁵R¹⁶, —C(Z)NR⁹R¹⁰, —C(Z)R¹¹, —S(O)₂NR⁹R¹⁰, and —S(O)ₙR¹²;
R⁶ is selected from the group consisting of hydrogen, optionally substituted lower alkyl, optionally substituted lower alkenyl, provided, however, that when R⁶ is optionally substituted lower alkenyl, no alkene carbon thereof is bound to the N of NR⁶, optionally substituted lower alkynyl, provided, however, that when R⁶ is optionally substituted lower alkynyl, no alkyne carbon thereof is bound to the N of NR⁶, optionally substituted cycloalkyl, optionally substituted heterocycloalkyl, optionally substituted aryl, optionally substituted heteroaryl, —C(Z)NR⁹R¹⁰, —C(Z)R¹¹, —S(O)₂NR⁹R¹⁰, and —S(O)₂R¹²;
R⁹ and R¹⁰ at each occurrence are independently selected from the group consisting of hydrogen, optionally substituted lower alkyl, optionally substituted lower alkenyl, provided, however, that when R⁹ and/or R¹⁰ are optionally substituted lower alkenyl, no alkene carbon thereof is bound to the N of NR⁹R¹⁰, optionally substituted lower alkynyl, provided, however, that when R⁹ and/or R¹⁰ are optionally substituted lower alkynyl, no alkyne carbon thereof is bound to the N of NR⁹R¹⁰, optionally substituted cycloalkyl, optionally substituted heterocycloalkyl, optionally substituted aryl, and optionally substituted heteroaryl;
R¹¹ at each occurrence is independently selected from the group consisting of optionally substituted lower alkyl, optionally substituted lower alkenyl, provided, however, that when R¹¹ is optionally substituted lower alkenyl, no alkene carbon thereof is bound to the C(Z) of C(Z)R¹¹, optionally substituted lower alkynyl, provided, however, that when R¹¹ is optionally substituted lower alkynyl, no alkyne carbon thereof is bound to the C(Z) of C(Z)R¹¹, optionally substituted cycloalkyl, optionally substituted heterocycloalkyl, optionally substituted aryl, optionally substituted heteroaryl, and —OR¹⁴;
R¹² at each occurrence is independently selected from the group consisting of optionally substituted lower alkyl, optionally substituted lower alkenyl, provided, however, that when R¹² is optionally substituted lower alkenyl, no alkene carbon thereof is bound to the S(O)ₙ of S(O)ₙR¹², optionally substituted lower alkynyl, provided, however, that when R¹² is optionally substituted lower alkynyl, no alkyne carbon thereof is bound to the S(O)ₙ of S(O)ₙNR¹², optionally substituted cycloalkyl, optionally substituted heterocycloalkyl, optionally substituted aryl, and optionally substituted heteroaryl;
R¹³ at each occurrence is independently selected from the group consisting of hydrogen, optionally substituted lower alkyl, optionally substituted lower alkenyl, provided, however, that when R¹³ is optionally substituted lower alkenyl, no alkene carbon thereof is bound to the O of OR¹³, optionally substituted lower alkynyl, provided, however, that when R¹³ is optionally substituted lower alkynyl, no alkyne carbon thereof is bound to the O of OR¹³, optionally substituted cycloalkyl, optionally substituted heterocycloalkyl, optionally substituted aryl, optionally substituted heteroaryl, —C(Z)R¹¹ and —C(Z)NR⁹R¹⁰;
R¹⁴ at each occurrence is independently selected from the group consisting of hydrogen, optionally substituted lower alkyl, optionally substituted lower alkenyl, provided, however, that when R¹⁴ is optionally substituted lower alkenyl, no alkene carbon thereof is bound to the S of SR¹⁴ or the 0 of OR¹⁴, optionally substituted lower alkynyl, provided, however, that when R¹⁴ is optionally substituted lower alkynyl, no alkyne carbon thereof is bound to the S of SR¹⁴ or the O of OR¹⁴, optionally substituted cycloalkyl, optionally substituted heterocycloalkyl, optionally substituted aryl, and optionally substituted heteroaryl;
R¹⁵ and R¹⁶ at each occurrence are independently selected from the group consisting of hydrogen, optionally substituted lower alkyl, optionally substituted lower alkenyl, provided, however, that when R¹⁵ and/or R¹⁶ are optionally substituted lower alkenyl, no alkene carbon thereof is bound to the N of NR¹⁵R¹⁶, optionally substituted lower alkynyl, provided, however, that when R¹⁵ and/or R¹⁶ are optionally substituted lower alkynyl, no alkyne carbon thereof is bound to the N of NR¹⁵R¹⁶, optionally substituted cycloalkyl, optionally substituted heterocycloalkyl, optionally substituted aryl, optionally substituted heteroaryl, —C(Z)R¹¹, —C(Z)NR⁹R¹⁰, —S(O)₂R¹², and —S(O)₂NR⁹R¹⁰;
R¹⁷ is selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁷ is lower alkyl, any substitution on the alkyl carbon bound to the O of OR¹⁷ is fluoro;
R¹⁸ and R¹⁹ are independently selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁸ and/or R¹⁹ is lower alkyl, any substitution on the alkyl carbon bound to the N of NR¹⁸R¹⁹ is fluoro; or
R¹⁸ and R¹⁹ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
n is 1 or 2; and
Z is O or S, provided, however, that when R² is —C(O)R¹¹, T and X are either N or CH, and provided the compound is not

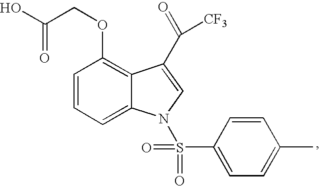

or an ester thereof.

In one embodiment of compounds of Formula I, no more than two of T, U, V, W, X and Y are nitrogen. In another embodiment, R⁵ is selected from the group consisting of hydrogen, halogen, optionally fluoro substituted lower alkyl, optionally fluoro substituted lower alkylthio, and optionally fluoro substituted lower alkoxy. In one embodiment, when Q is —NR⁶—, R⁶ is hydrogen or optionally substituted lower alkyl, preferably hydrogen or lower alkyl optionally substituted with fluoro, —OH, lower alkoxy, or lower alkylthio, provided that any substitution of the carbon that is bound to the N of —NR⁶— is fluoro. In one embodiment, R² is —C(Z)NR⁹R¹⁰, —C(Z)R¹¹, —S(O)₂NR⁹R¹⁰, or —S(O)₂R¹², preferably —S(O)₂R¹². In one embodiment, one of T, U, W, X and Y is CR⁵, the others of T, U, W, X and Y are CH, and Q is bound to V. In one embodiment, two of T, U, W, X and Y are CR⁵, the others of T, U, W, X and Y are CH, and Q is bound to V. In one embodiment, three of T, U, W, X and Y are CR⁵, the others of T, U, W, X and Y are CH, and Q is bound to V. In one embodiment, T, U, W, X and Y are CH and Q is bound to V. In one embodiment, one of T, V, W, X and Y is CR⁵, the others of T, V, W, X and Y are CH, and Q is bound to U. In one embodiment, two of T, V, W, X and Y are CR⁵, the others of T, V, W, X and Y are CH, and Q is bound to U. In one embodiment, three of T, V, W, X and Y are CR⁵, the others of T, V, W, X and Y are CH, and Q is bound to U. In one embodiment, T, V, W, X and Y are CH, and Q is bound to U. In one embodiment, T, U, W and X are CR⁵, Y is N, and Q is bound to V. In one embodiment, one of T, U, W and X is CR⁵, the others of T, U, W and X are CH, Y is N, and Q is bound to V. In one embodiment, T, U, W and X are CH, Y is N, and Q is bound to V. In one embodiment, T, V, W and X are CR⁵, Y is N, and Q is bound to U. In one embodiment, one of T, V, W and X is CR⁵, the others of T, V, W and X are CH, Y is N, and Q is bound to U. In one embodiment, T, V, W and X are CH, Y is N, and Q is bound to U.

In some embodiments, compounds of Formula I have a structure selected from Formula Ia or Ib as shown below:

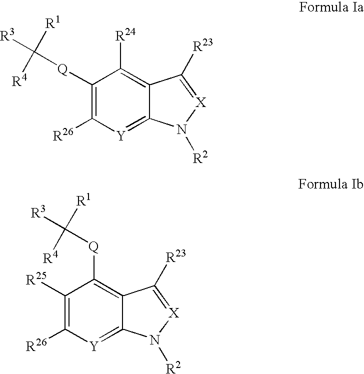

all salts, prodrugs, tautomers and isomers thereof
wherein:
T is CR⁵, wherein R⁵ is R²³;
W is CR⁵, wherein R⁵ is R²⁶;
when V is C bound to Q, U is CR⁵, wherein R⁵ is R²⁴;
when U is C bound to Q, V is CR⁵, wherein R⁵ is R²⁵;
X, Y, Q, R¹, R³, and R⁴ are as defined in Formula I above;
R² is selected from the group consisting of —C(Z)NR⁹R¹⁰, —C(Z)R¹¹, —S(O)₂NR⁹R¹⁰, and —S(O)₂R¹²;
R²³, R²⁴, R²⁵, and R²⁶ are independently selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OR¹³, —SR¹⁴, —NR¹⁵R¹⁶, —C(Z)NR⁹R¹⁰, —C(Z)R¹¹, —S(O)₂NR⁹R¹⁰, and —S(O)ₙR¹²;
n, Z, R⁹, R¹⁰, R¹¹, R¹², R¹³, R¹⁴, R¹⁵, and R¹⁶ are as defined in Formula I.

In one embodiment of compounds of Formula Ia or Ib, R² is —S(O)₂R¹². In one embodiment of compounds of Formula Ia, X is CH, one of R²³, R²⁴, and R²⁶ is selected from the group consisting of halogen, optionally fluoro substituted lower alkyl, optionally fluoro substituted lower alkylthio, and optionally fluoro substituted lower alkoxy, and the others of R²³, R²⁴, and R²⁶ are hydrogen. In one embodiment of compounds of Formula Ia, X and Y are CH, one of R²³, R²⁴, and R²⁶ is selected from the group consisting of halogen, optionally fluoro substituted lower alkyl, optionally fluoro substituted lower alkylthio, and optionally fluoro substituted lower alkoxy, and the others of R²³, R²⁴, and R²⁶ are hydrogen.

In one embodiment of compounds of Formula Ib, X is CH, one of R²³, R²⁵, and R²⁶ is selected from the group consisting of halogen, optionally fluoro substituted lower alkyl, optionally fluoro substituted lower alkylthio, and optionally fluoro substituted lower alkoxy, and the others of R²³, R²⁵, and R²⁶ are hydrogen. In one embodiment of compounds of Formula Ib, X and Y are CH, one of R²³, R²⁵, and R²⁶ is selected from the group consisting of halogen, optionally fluoro substituted lower alkyl, optionally fluoro substituted lower alkylthio, and optionally fluoro substituted lower alkoxy, and the others of R²³, R²⁵, and R²⁶ are hydrogen.

In one embodiment of compounds of Formula Ia or Ib, when Q is —NR⁶—, R⁶ is hydrogen or optionally substituted lower alkyl, preferably hydrogen or lower alkyl optionally substituted with fluoro, —OH, lower alkoxy, or lower alkylthio, provided that any substitution of the carbon that is bound to the N of —NR⁶— is fluoro.

In some embodiments, compounds of Formula I have a structure of Formula Ic as shown below:

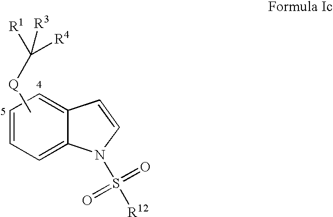

wherein T is CR⁵, and R⁵ is H, W is CR⁵, and R⁵ is H, Y is CR⁵, and R⁵ is H, X is CR⁵, and R⁵ is H, when V is C bound to Q, U is CR⁵ and R⁵ is H, when U is C bound to Q, V is CR⁵ and R⁵ is H, and R¹, R³, R⁴, R¹², and Q are as defined in Formula I above. In one embodiment of compounds of Formula Ic, Q is O. In one embodiment, R³ and R⁴ are H, and Q is O. In one embodiment, Q is CH₂. In one embodiment, R³ and R⁴ are H, and Q is CH₂.

In another aspect, the invention provides compounds of Formula II having the structure shown below:

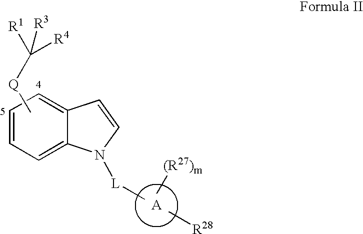

all salts, prodrugs, tautomers and isomers thereof
wherein:
Q is —O—, —S—, —NR⁶—, or —CR⁷R⁸—, and wherein Q is bound to the carbon atom at either the 4 or the 5 position of indole with the other carbon atom at the 4 or the 5 position being substituted with hydrogen;
R¹ is selected from the group consisting of —C(O)OR¹⁷, —C(O)NR¹⁸R¹⁹, and a carboxylic acid isostere;
R³, R⁴, R⁷ and R⁸ are independently selected from the group consisting of hydrogen, optionally substituted lower alkyl, optionally substituted cycloalkyl, optionally substituted heterocycloalkyl, optionally substituted aryl, and optionally substituted heteroaryl; or
any two of R³, R⁴, R⁷ and R⁸ may combine to form optionally substituted 3-7 membered monocyclic cycloalkyl or optionally substituted 3-7 membered monocyclic heterocycloalkyl;
A is a monocyclic or bicyclic ring selected from the group consisting of cycloalkyl, heterocycloalkyl, aryl, and heteroaryl;
L is selected from the group consisting of —C(Z)NR²⁹—, —C(Z)-, —S(O)₂NR²⁹—, and —S(O)₂—, attached to A at any available atom to produce a stable compound;
R²⁷ at each occurrence is independently selected from the group consisting of halogen, —OH, lower alkyl, lower alkoxy, and lower alkylthio, wherein lower alkyl and the lower alkyl chains of lower alkoxy and lower alkylthio are optionally substituted with fluoro, —OH, lower alkoxy, or lower alkylthio, provided that any substitution of the carbon bound to the O of lower alkoxy or S of lower alkylthio is fluoro;
R²⁸ is selected from the group consisting of hydrogen, halogen, cyano, nitro, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OR¹³, —SR⁴, —NR¹⁵R¹⁶, —C(Z)NR⁹R¹⁰, —C(Z)R¹¹, —S(O)₂NR⁹R¹⁰, and —S(O)ₙR², attached to A at any available atom to produce a stable compound;
R⁶ is selected from the group consisting of hydrogen, optionally substituted lower alkyl, optionally substituted lower alkenyl, provided, however, that when R⁶ is optionally substituted lower alkenyl, no alkene carbon thereof is bound to the N of NR⁶, optionally substituted lower alkynyl, provided, however, that when R⁶ is optionally substituted lower alkynyl, no alkyne carbon thereof is bound to the N of NR⁶, optionally substituted cycloalkyl, optionally substituted heterocycloalkyl, optionally substituted aryl, optionally substituted heteroaryl, —C(Z)NR⁹R¹⁰, —C(Z)R¹¹, —S(O)₂NR⁹R¹⁰, and —S(O)₂R¹²;
R⁹ and R¹⁰ at each occurrence are independently selected from the group consisting of hydrogen, optionally substituted lower alkyl, optionally substituted lower alkenyl, provided, however, that when R⁹ and/or R¹⁰ are optionally substituted lower alkenyl, no alkene carbon thereof is bound to the N of NR⁹R¹⁰, optionally substituted lower alkynyl, provided, however, that when R⁹ and/or R¹⁰ are optionally substituted lower alkynyl, no alkyne carbon thereof is bound to the N of NR⁹R¹⁰, optionally substituted cycloalkyl, optionally substituted heterocycloalkyl, optionally substituted aryl, and optionally substituted heteroaryl;
R¹¹ at each occurrence is independently selected from the group consisting of optionally substituted lower alkyl, optionally substituted lower alkenyl, provided, however, that when R¹¹ is optionally substituted lower alkenyl, no alkene carbon thereof is bound to the C(Z) of C(Z)R¹¹, optionally substituted lower alkynyl, provided, however, that when R¹¹ is optionally substituted lower alkynyl, no alkyne carbon thereof is bound to the C(Z) of C(Z)R¹¹, optionally substituted cycloalkyl, optionally substituted heterocycloalkyl, optionally substituted aryl, optionally substituted heteroaryl, and —OR¹⁴;
R¹² at each occurrence is independently selected from the group consisting of optionally substituted lower alkyl, optionally substituted lower alkenyl, provided, however, that when R¹² is optionally substituted lower alkenyl, no alkene carbon thereof is bound to the S(O)ₙ of S(O)ₙR¹², optionally substituted lower alkynyl, provided, however, that when R¹² is optionally substituted lower alkynyl, no alkyne carbon thereof is bound to the S(O)ₙ of S(O)ₙR¹², optionally substituted cycloalkyl, optionally substituted heterocycloalkyl, optionally substituted aryl, and optionally substituted heteroaryl;
R¹³ at each occurrence is independently selected from the group consisting of hydrogen, optionally substituted lower alkyl, optionally substituted lower alkenyl, provided, however, that when R¹³ is optionally substituted lower alkenyl, no alkene carbon thereof is bound to the O of OR¹³, optionally substituted lower alkynyl, provided, however, that when R¹³ is optionally substituted lower alkynyl, no alkyne carbon thereof is bound to the O of OR¹³, optionally substituted cycloalkyl, optionally substituted heterocycloalkyl, optionally substituted aryl, optionally substituted heteroaryl, —C(Z)R¹¹ and —C(Z)NR⁹R¹⁰;
R¹⁴ at each occurrence is independently selected from the group consisting of hydrogen, optionally substituted lower alkyl, optionally substituted lower alkenyl, provided, however, that when R¹⁴ is optionally substituted lower alkenyl, no alkene carbon thereof is bound to the S of SR¹⁴ or the O of OR¹⁴ optionally substituted lower alkynyl, provided, however, that when R¹⁴ is optionally substituted lower alkynyl, no alkyne carbon thereof is bound to the S of SR¹⁴ or the O of OR¹⁴, optionally substituted cycloalkyl, optionally substituted heterocycloalkyl, optionally substituted aryl, and optionally substituted heteroaryl;
R¹⁵ and R¹⁶ at each occurrence are independently selected from the group consisting of hydrogen, optionally substituted lower alkyl, optionally substituted lower alkenyl, provided, however, that when R¹⁵ and/or R¹⁶ are optionally substituted lower alkenyl, no alkene carbon thereof is bound to the N of NR¹⁵R¹⁶, optionally substituted lower alkynyl, provided, however, that when R¹⁵ and/or R¹⁶ are optionally substituted lower alkynyl, no alkyne carbon thereof is bound to the N of NR¹⁵R¹⁶, optionally substituted cycloalkyl, optionally substituted heterocycloalkyl, optionally substituted aryl, optionally substituted heteroaryl, —C(Z)R¹¹, —C(Z)NR⁹R¹⁰, —S(O)₂R¹², and —S(O)₂NR⁹R¹⁰;
R¹⁷ is selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁷ is lower alkyl, any substitution on the alkyl carbon bound to the 0 of OR¹⁷ is fluoro;
R¹⁸ and R¹⁹ are independently selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁸ and/or R¹⁹ is lower alkyl, any substitution on the alkyl carbon bound to the N of NR¹⁸R¹⁹ is fluoro; or
R¹⁸ and R¹⁹ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R²⁹ is selected from the group consisting of hydrogen, optionally substituted lower alkyl, optionally substituted lower alkenyl, provided, however, that when R²⁹ is optionally substituted lower alkenyl, no alkene carbon thereof is bound to the N of NR²⁹, optionally substituted lower alkynyl, provided, however, that when R²⁹ is optionally substituted lower alkynyl, no alkyne carbon thereof is bound to the N of NR²⁹, optionally substituted cycloalkyl, optionally substituted heterocycloalkyl, optionally substituted aryl, and optionally substituted heteroaryl;
n is 1 or 2;
m is 0, 1, or 2; and
Z is O or S.

In one embodiment of compounds of Formula II, A is a monocyclic aryl or monocyclic heteroaryl ring. In one embodiment, A is a monocyclic heteroaryl ring. In one embodiment, R²⁸ is selected from the group consisting of optionally substituted cycloalkyl, optionally substituted heterocycloalkyl, optionally substituted aryl, optionally substituted heteroaryl, —OR¹³, —SR¹⁴, —NR¹⁵R¹⁶, —C(Z)NR⁹R¹⁰, —C(Z)R¹¹, —S(O)₂NR⁹R¹⁰, and —S(O)ₙR¹², further wherein one of R⁹ and R¹⁰, one of R¹⁵ and R¹⁶, R¹¹, R¹², R¹³ and R¹⁴ are selected from the group consisting of optionally substituted cycloalkyl, optionally substituted heterocycloalkyl, optionally substituted aryl, and optionally substituted heteroaryl. In one embodiment Q is O. In one embodiment, R³ and R⁴ are H and Q is O. In one embodiment Q is CH₂. In one embodiment, R³ and R⁴ are H and Q is CH₂. In one embodiment, when Q is —NR⁶—, R⁶ is hydrogen or optionally substituted lower alkyl, preferably hydrogen or lower alkyl optionally substituted with fluoro, —OH, lower alkoxy, or lower alkylthio, provided that any substitution of the carbon that is bound to the N of —NR⁶— is fluoro.

In some embodiments involving compounds of Formulae I, Ia, Ib, Ic, and II, the compounds have a structure in which the bicyclic core has one of the following structures:

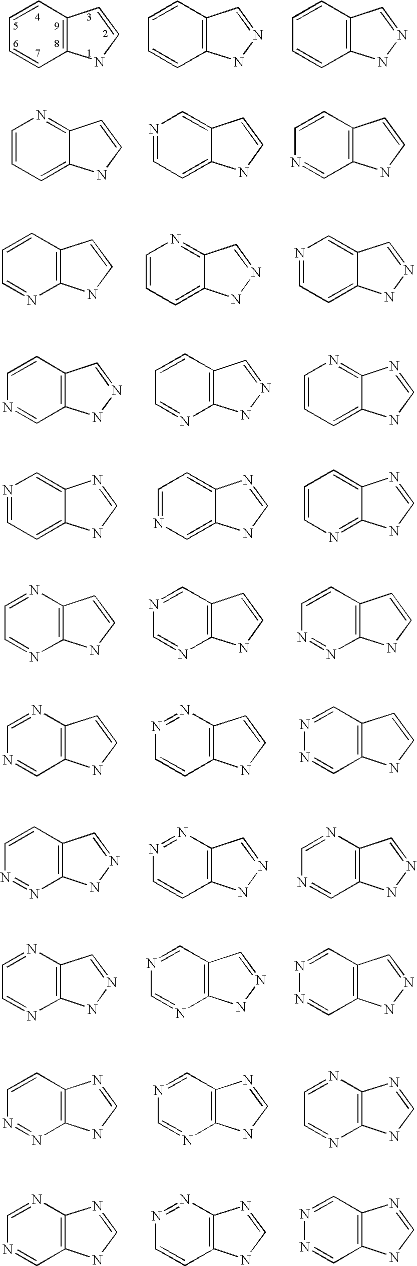

Unless indicated to the contrary, reference to positional numbering of bicyclic structures provided herein is based on the numbering of indole as shown above.

Thus, in some embodiments involving compounds of Formulae I, Ia, Ib, Ic, and II, the compound includes a bicyclic core as shown above. Such compounds can include substitutents as described for all embodiments of Formulae I, Ia, Ib, Ic, and II, with the understanding that ring nitrogens, other than the nitrogen corresponding to position 1 of the indole structure, are unsubstituted. In some embodiments, the compounds have one of the bicyclic cores shown above and substitution selections as shown herein for compounds having an indole core.

In some embodiments of the above compounds, compounds are excluded where N (except where N is a heteroaryl ring atom), O, or S is bound to a carbon that is also bound to N (except where N is a heteroaryl ring atom), O, or S; or where N (except where N is a heteroaryl ring atom), O, C(S), C(O), or S(O)ₙ (n is 0-2) is bound to an alkene carbon of a lower alkenyl group or bound to an alkyne carbon of a lower alkynyl group; accordingly, in some embodiments compounds that include linkages such as the following are excluded from the present invention: —NR—CH₂—NR—, —O—CH₂—NR—, —S—CH₂—NR—, —NR—CH₂—O—, —O—CH₂—O—, —S—CH₂—O—, —NR—CH₂—S—, —O—CH₂—S—, —S—CH₂—S—, —NR—CH═CH—, —CH═CH—NR—, —NR—C≡C—, —C≡C—NR—, —O—CH═CH—, —CH═CH—O—, —O—C≡C—, —C≡C—O—, —S(O)₀₋₂—CH═CH—, —CH═CH—S(O)₀₋₂—, —S(O)₀₋₂—C≡C—, —C≡C—S(O)₀₋₂—, —C(O)—CH═CH—, —CH═CH—C(O)—, —C≡C—C(O)—, —C(O)—C≡C—, —C(S)—CH═CH—, —CH═CH—C(S)—, —C≡C—C(S)—, or —C(S)—C≡C—.

Reference to compounds of Formula I herein includes specific reference to sub-groups and species of compounds of Formula I described herein (including all embodiments as described above or in the examples, e.g. reference to Formula I includes reference to Formulae Ia, Ib, and Ic) unless indicated to the contrary. Reference to compounds of Formula II herein includes specific reference to sub-groups and species of compounds of Formula II described herein (including all embodiments as described above or in the examples) unless indicated to the contrary. In specifying a compound or reference to compounds of Formulae I, Ia, Ib, Ic or II, unless clearly indicated to the contrary, specification of such compound(s) includes pharmaceutically acceptable salts of the compound(s).

Another aspect of the invention concerns novel use of compounds of Formula I for the treatment of diseases associated with PPARs. Another aspect of the invention provides novel compounds of Formula I. Another aspect of the invention concerns novel use of compounds of Formula II for the treatment of diseases associated with PPARs. Another aspect of the invention provides novel compounds of Formula II.

Another aspect of this invention provides compositions that include a therapeutically effective amount of a compound of Formula I and at least one pharmaceutically acceptable carrier, excipient, and/or diluent. The composition can include a plurality of different pharmacalogically active compounds, including one or more compounds of Formula I. Another aspect of this invention provides compositions that include a therapeutically effective amount of a compound of Formula II and at least one pharmaceutically acceptable carrier, excipient, and/or diluent. The composition can include a plurality of different pharmacalogically active compounds, including one or more compounds of Formula II.

In another aspect, compounds of Formulae I, Ia, Ib, Ic, and II can be used in the preparation of a medicament for the treatment of a PPAR-mediated disease or condition or a disease or condition in which modulation of a PPAR provides a therapeutic benefit. In a further aspect, the disease or condition is selected from the group consisting of weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer.

In another aspect, the invention provides kits that include a compound or composition as described herein. In some embodiments, the compound or composition is packaged, e.g., in a vial, bottle, flask, which may be further packaged, e.g., within a box, envelope, or bag; the compound or composition is approved by the U.S. Food and Drug Administration or similar regulatory agency for administration to a mammal, e.g., a human; the compound or composition is approved for administration to a mammal, e.g., a human for a PPAR-mediated disease or condition; the kit includes written instructions or other indication that the compound or composition is suitable or approved for administration to a mammal, e.g., a human, for a PPAR-mediated disease or condition; the composition is packaged in unit does or single dose form, e.g., single dose pills, capsules, or the like. In some embodiments, the compound or composition of the kit is approved for a medical indication selected from the group consisting of obesity, overweight condition, hyperlipidemia, associated diabetic dyslipidemia and mixed dyslipidemia, mixed dyslipidemia, hypoalphalipoproteinemia, Syndrome X, Type II diabetes mellitus, Type I diabetes, hyperinsulinemia, impaired glucose tolerance, insulin resistance, a diabetic complication of neuropathy, nephropathy, retinopathy or cataracts, hypertension, coronary heart disease, heart failure, hypercholesterolemia, inflammation, thrombosis, congestive heart failure, cardiovascular disease, atherosclerosis, arteriosclerosis, hypertriglyceridemia, eczema, psoriasis, cancer, conditions associated with the lung and gut, regulation of appetite and food intake in subjects suffering from disorders such as obesity, anorexia bulimia and anorexia nervosa, neurodegenerative diseases, Alzheimer's disease, Parkinson's disease, amyotrophic lateral sclerosis, rheumatoid arthritis, inflammatory bowel syndrome, Crohn's disease, multiple sclerosis, infertility, asthma, chronic obstructive pulmonary disease, and macular degeneration.

In another aspect, the invention provides a method of treating or prophylaxis of a disease or condition in an animal subject, e.g., a PPAR-mediated disease or condition or a disease or condition in which modulation of a PPAR provides a therapeutic benefit, by administering to the subject a therapeutically effective amount of a compound of Formula I, a prodrug of such compound, or a pharmaceutically acceptable salt of such compound or prodrug. The compound can be administered alone or can be administered as part of a pharmaceutical composition. In one aspect, the method involves administering to the subject an effective amount of a compound of Formula I, in combination with one or more other therapies for the disease or condition. In another aspect, the invention provides a method of treating or prophylaxis of a disease or condition in an animal subject, e.g., a PPAR-mediated disease or condition or a disease or condition in which modulation of a PPAR provides a therapeutic benefit, by administering to the subject a therapeutically effective amount of a compound of Formula II, a prodrug of such compound, or a pharmaceutically acceptable salt of such compound or prodrug. The compound can be administered alone or can be administered as part of a pharmaceutical composition. In one aspect, the method involves administering to the subject an effective amount of a compound of Formula II, in combination with one or more other therapies for the disease or condition.

In another aspect, the invention provides a method of treating or prophylaxis of a PPAR-mediated disease or condition or a disease or condition in which modulation of a PPAR provides a therapeutic benefit, wherein the method involves administering to the subject a therapeutically effective amount of a composition including a compound of Formula I. In another aspect, the invention provides a method of treating or prophylaxis of a PPAR-mediated disease or condition or a disease or condition in which modulation of a PPAR provides a therapeutic benefit, wherein the method involves administering to the subject a therapeutically effective amount of a composition including a compound of Formula II.

In aspects and embodiments involving treatment or prophylaxis of a disease or conditions, the disease or condition is selected from the group consisting of weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer.

In some embodiments of aspects involving compounds of Formulae I, Ia, Ib, Ic, and II, the compound is specific for any one or any two of PPARα, PPARδ and PPARδ, e.g. specific for PPARα; specific for PPARδ; specific for PPARγ; specific for PPARα and PPARδ; specific for PPARα and PPARγ; or specific for PPARδ and PPARγ. Such specificity means that the compound has at least 5-fold greater activity (preferably at least 5-, 10-, 20-, 50-, or 100-fold or more greater activity) on the specific PPAR(s) than on the other PPAR(s), where the activity is determined using a biochemical assay suitable for determining PPAR activity, e.g., any assay known to one skilled in the art or as described herein. In another embodiment, compounds have significant activity on all three of PPARα, PPARδ, and PPARγ.

In some embodiments, a compound of Formulae I, Ia, Ib, Ic, and II, will have an EC₅₀ of less than 100 nM, less than 50 nM, less than 20 nM, less than 10 nM, less than 5 nM, or less than 1 nM with respect to at least one of PPARα, PPARγ and PPARδ as determined in a generally accepted PPAR activity assay. In one embodiment, a compound of Formula I, will have an EC₅₀ of less than 100 nM, less than 50 nM, less than 20 nM, less than 10 nM, less than 5 nM, or less than 1 nM with respect to at least any two of PPARα, PPARγ and PPARδ. In one embodiment, a compound of Formula I will have an EC₅₀ of less than 100 nM, less than 50 nM, less than 20 nM, less than 10 nM, less than 5 nM, or less than 1 nM with respect to all three of PPARα, PPARγ and PPARδ. In one embodiment, a compound of Formula II, will have an EC₅₀ of less than 100 nM, less than 50 nM, less than 20 nM, less than 10 nM, less than 5 nM, or less than 1 nM with respect to at least any two of PPARα, PPARγ and PPARδ. In one embodiment, a compound of Formula II will have an EC₅₀ of less than 100 nM, less than 50 nM, less than 20 nM, less than 10 nM, less than 5 nM, or less than 1 nM with respect to all three of PPARα, PPARγ and PPARδ. Further to any of the above embodiments, a compound of the invention may be a specific agonist of any one of PPARα, PPARγ and PPARδ, or any two of PPARα, PPARγ and PPARδ. A specific agonist of one of PPARα, PPARγ and PPARδ is such that the EC₅₀ for one of PPARα, PPARγ and PPARδ will be at least about 5-fold, also 10-fold, also 20-fold, also 50-fold, or at least about 100-fold less than the EC₅₀ for the other two of PPARα, PPARγ and PPARδ. A specific agonist of two of PPARα, PPARγ and PPARδ is such that the EC₅₀ for each of two of PPARα, PPARγ and PPARδ will be at least about 5-fold, also 10-fold, also 20-fold, also 50-fold, or at least about 100-fold less than the EC₅₀ for the other of PPARα, PPARγ and PPARδ.

In some embodiments of the invention, the compounds of Formulae I, Ia, Ib, Ic, and II active on PPARs also have desirable pharmacologic properties. In some embodiments the desired pharmacologic property is PPAR pan-activity, PPAR selectivity for any individual PPAR (i.e., PPARα, PPARδ, or PPARγ), selectivity on any two PPARs (i.e., PPARα and PPARδ, PPARα and PPARγ, or PPARδ and PPARγ), or any one or more of serum half-life longer than 2 hr, also longer than 4 hr, also longer than 8 hr, aqueous solubility, and oral bioavailability more than 10%, also more than 20%.

Additional embodiments will be apparent from the Detailed Description and from the claims.

### DETAILED DESCRIPTION OF THE INVENTION

As indicated in the Summary above, the present invention concerns the peroxisome proliferator-activated receptors (PPARs), which have been identified in humans and other mammals. A group of compounds have been identified, corresponding to Formula I, Ia, Ib, Ic or II, that are active on one or more of the PPARs, in particular compounds that are active on one or more human PPARs. Such compounds can be used as agonists on PPARs, including agonists of at least one of PPARα, PPARδ, and PPARγ, as well as dual PPAR agonists and pan-agonist, such as agonists of both PPARα and PPARγ, both PPARα and PPARδ, both PPARγ and PPARδ, or agonists of PPARα, PPARγ and PPARδ.

As used herein the following definitions apply unless otherwise indicated:

“Halogen”—alone or in combination refers to all halogens, that is, chloro (Cl), fluoro (F), bromo (Br), or iodo (I).

“Hydroxyl” or “hydroxy” refers to the group —OH.

“Thiol” refers to the group —SH.

“Lower alkyl” alone or in combination means an alkane-derived radical containing from 1 to 6 carbon atoms (unless specifically defined) that includes a straight chain alkyl or branched alkyl. The straight chain or branched alkyl group is attached at any available point to produce a stable compound. In many embodiments, a lower alkyl is a straight or branched alkyl group containing from 1-6, 1-4, or 1-2, carbon atoms, such as methyl, ethyl, propyl, isopropyl, butyl, t-butyl, and the like. “Substituted lower alkyl” denotes lower alkyl that is independently substituted, unless indicated otherwise, with one or more, preferably 1, 2, 3, 4 or 5, also 1, 2, or 3 substituents, attached at any available atom to produce a stable compound, wherein the substituents are selected from the group consisting of —F, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{e}, and —R^{f}. Furthermore, possible substitutions include subsets of these substitutions, such as are indicated herein, for example, in the description of compounds of Formulae I, Ia, Ib, Ic, and II attached at any available atom to produce a stable compound. For example “fluoro substituted lower alkyl” denotes a lower alkyl group substituted with one or more fluoro atoms, such as perfluoroalkyl, where preferably the lower alkyl is substituted with 1, 2, 3, 4 or 5 fluoro atoms, also 1, 2, or 3 fluoro atoms. It is understood that substitutions are attached at any available atom to produce a stable compound, when optionally substituted lower alkyl is an R group of a moiety such as —OR (e.g. alkoxy), —SR (e.g. thioalkyl), —NHR (e.g. alkylamino), —C(O)NHR, and the like, substitution of the lower alkyl R group is such that substitution of the alkyl carbon bound to any O, S, or N of the moiety (except where N is a heteroaryl ring atom) excludes substituents that would result in any O, S, or N of the substituent (except where N is a heteroaryl ring atom) being bound to the alkyl carbon bound to any O, S, or N of the moiety.

“Lower alkenyl” alone or in combination means a straight or branched hydrocarbon containing 2-6 carbon atoms (unless specifically defined) and at least one, preferably 1-3, more preferably 1-2, most preferably one, carbon to carbon double bond. Carbon to carbon double bonds may be contained within either a straight chain or branched portion. Examples of lower alkenyl groups include ethenyl, propenyl, isopropenyl, butenyl, and the like. “Substituted lower alkenyl” denotes lower alkenyl that is independently substituted, unless indicated otherwise, with one or more, preferably 1, 2, 3, 4 or 5, also 1, 2, or 3 substituents, attached at any available atom to produce a stable compound, wherein the substituents are selected from the group consisting of —F, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{d}, and —R^{f}. Further, possible substitutions include subsets of these substitutions, such as are indicated herein, for example, in the description of compounds of Formulae I, Ia, Ib, Ic, and II attached at any available atom to produce a stable compound. It is understood that substitutions are attached at any available atom to produce a stable compound, substitution of lower alkenyl groups are such that F, C(O), C(S), C(NH), S(O), S(O)₂, O, S, or N (except where N is a heteroaryl ring atom), are not bound to an alkene carbon thereof. Further, where lower alkenyl is a substituent of another moiety or an R group of a moiety such as —OR, —NHR, —C(O)R, and the like, substitution of the moiety is such that any C(O), C(S), S(O), S(O)₂, O, S, or N thereof (except where N is a heteroaryl ring atom) are not bound to an alkene carbon of the lower alkenyl substituent or R group. Further, where lower alkenyl is a substituent of another moiety or an R group of a moiety such as —OR, —NHR, —C(O)NHR, and the like, substitution of the lower alkenyl R group is such that substitution of the alkenyl carbon bound to any O, S, or N of the moiety (except where N is a heteroaryl ring atom) excludes substituents that would result in any O, S, or N of the substituent (except where N is a heteroaryl ring atom) being bound to the alkenyl carbon bound to any O, S, or N of the moiety. An “alkenyl carbon” refers to any carbon within a lower alkenyl group, whether saturated or part of the carbon to carbon double bond. An “alkene carbon” refers to a carbon within a lower alkenyl group that is part of a carbon to carbon double bond. “C₃₋₆ alkenyl” denotes lower alkenyl containing 3-6 carbon atoms. A “substituted C₃₋₆ alkenyl” denotes optionally substituted lower alkenyl containing 3-6 carbon atoms.

“Lower alkynyl” alone or in combination means a straight or branched hydrocarbon containing 2-6 carbon atoms (unless specifically defined) containing at least one, preferably one, carbon to carbon triple bond. Examples of lower alkynyl groups include ethynyl, propynyl, butynyl, and the like. “Substituted lower alkynyl” denotes lower alkynyl that is independently substituted, unless indicated otherwise, with one or more, preferably 1, 2, 3, 4 or 5, also 1, 2, or 3 substituents, attached at any available atom to produce a stable compound, wherein the substituents are selected from the group consisting of —F, —NO₂, —CN, —OR^{a}, —SR^{a}, —OC(O)R^{a}, —OC(S)R^{a}, —C(O)R^{a}, —C(S)R^{a}, C(O)OR^{a}C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{d}, and —R^{f}. Further, possible substitutions include subsets of these substitutions, such as are indicated herein, for example, in the description of compounds of Formulae I, Ia, Ib, Ic, and II attached at any available atom to produce a stable compound. It is understood that substitutions are attached at any available atom to produce a stable compound, substitution of lower alkynyl groups are such that F, C(O), C(S), C(NH), S(O), S(O)₂, O, S, or N (except where N is a heteroaryl ring atom) are not bound to an alkyne carbon thereof. Further, where lower alkynyl is a substituent of another moiety or an R group of a moiety such as —OR, —NHR, —C(O)R, and the like, substitution of the moiety is such that any C(O), C(S), S(O), S(O)₂, O, S, or N thereof (except where N is a heteroaryl ring atom) are not bound to an alkyne carbon of the lower alkynyl substituent or R group. Further, where lower alkynyl is a substituent of another moiety or an R group of a moiety such as —OR, —NHR, —C(O)NHR, and the like, substitution of the lower alkynyl R group is such that substitution of the alkynyl carbon bound to any O, S, or N of the moiety (except where N is a heteroaryl ring atom) excludes substituents that would result in any O, S, or N of the substituent (except where N is a heteroaryl ring atom) being bound to the alkynyl carbon bound to any O, S, or N of the moiety. An “alkynyl carbon” refers to any carbon within an alkynyl group, whether saturated or part of the carbon to carbon triple bond. An “alkyne carbon” refers to a carbon within a lower alkynyl group that is part of a carbon to carbon triple bond. “C₃₋₆ alkynyl” denotes lower alkynyl containing 3-6 carbon atoms. A “substituted C₃₋₆ alkynyl” denotes optionally substituted lower alkynyl containing 3-6 carbon atoms.

“Carboxylic acid isostere” refers to a moiety selected from the group consisting of thiazolidine dione (i.e.

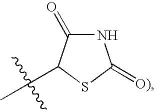

hydroxamic acid (i.e. —C(O)NHOH), acyl-cyanamide (i.e. —C(O)NHCN), tetrazole (i.e.

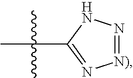

3- or 5hydroxy isoxazole (i.e.

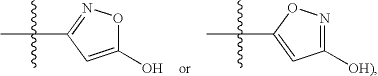

3- or 5-hydroxy isothiazole (i.e.

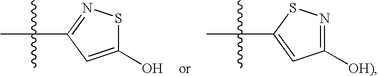

sulphonate (i.e. —S(O)₂OH), and sulfonamide (i.e. —S(O)₂NH₂). In functional terms, carboxylic acid isosteres mimic carboxylic acids by virtue of similar physical properties, including but not limited to molecular size, charge distribution or molecular shape. 3- or 5-hydroxy isoxazole or 3- or 5-hydroxy isothiazole may be optionally substituted with lower alkyl or lower alkyl substituted with 1, 2 or 3 substituents selected from the group consisting of fluoro, aryl and heteroaryl, wherein aryl or heteroaryl may further be optionally substituted with 1, 2, or 3 substituents selected from the group consisting of halogen, lower alkyl, fluoro substituted lower alkyl, lower alkoxy, fluoro substituted lower alkoxy, lower alkylthio, and fluoro substituted lower alkylthio. The nitrogen of the sulfonamide may be optionally substituted with a substituent selected from the group consisting of lower alkyl, fluoro substituted lower alkyl, acetyl (i.e. —C(O)CH₃), aryl and heteroaryl, wherein aryl or heteroaryl may further be optionally substituted with 1, 2, or 3 substituents selected from the group consisting of halogen, lower alkyl, fluoro substituted lower alkyl, lower alkoxy, fluoro substituted lower alkoxy, lower alkylthio, and fluoro substituted lower alkylthio.

“Aryl” alone or in combination refers to a monocyclic or bicyclic ring system containing aromatic hydrocarbons such as phenyl or naphthyl, which may be optionally fused with a cycloalkyl or heterocycloalkyl of preferably 5-7, more preferably 5-6, ring members. “Arylene” refers to a divalent aryl.

“Heteroaryl” alone or in combination refers to a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable compound is produced. Examples of heteroaryl groups include, but are not limited to, pyridinyl, pyridazinyl, pyrazinyl, quinoxalinyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. “Nitrogen containing heteroaryl” refers to heteroaryl wherein any heteroatoms are N. “Heteroarylene” refers to a divalent heteroaryl.

“Cycloalkyl” refers to saturated or unsaturated, non-aromatic monocyclic, bicyclic or tricyclic carbon ring systems of 3-10, also 3-8, more preferably 3-6, ring members per ring, such as cyclopropyl, cyclopentyl, cyclohexyl, adamantyl, and the like.

“Heterocycloalkyl” refers to a saturated or unsaturated non-aromatic cycloalkyl group having from 5 to 10 atoms in which from 1 to 3 carbon atoms in the ring are replaced by heteroatoms of O, S or N, and are optionally fused with benzo or heteroaryl of 5-6 ring members. Heterocycloalkyl is also intended to include oxidized S or N, such as sulfinyl, sulfonyl and N-oxide of a tertiary ring nitrogen. Heterocycloalkyl is also intended to include compounds in which one of the ring carbons is oxo substituted, i.e. the ring carbon is a carbonyl group, such as lactones and lactams. The point of attachment of the heterocycloalkyl ring is at a carbon or nitrogen atom such that a stable ring is retained. Examples of heterocycloalkyl groups include, but are not limited to, morpholino, tetrahydrofuranyl, dihydropyridinyl, piperidinyl, pyrrolidinyl, pyrrolidonyl, piperazinyl, dihydrobenzofuryl, and dihydroindolyl.

“Optionally substituted aryl”, “optionally substituted heteroaryl”, “optionally substituted cycloalkyl”, and “optionally substituted heterocycloalkyl”, refers to aryl, heteroaryl, cycloalkyl and heterocycloalkyl groups, respectively, which are optionally independently substituted, unless indicated otherwise, with one or more, preferably 1, 2, 3, 4 or 5, also 1, 2, or 3 substituents, attached at any available atom to produce a stable compound, wherein the substituents are selected from the group consisting of halogen, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —S(O)R^{a}, S(O)₂R^{a}, —C(O)NR^{a}R^{a}, C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{d}, —R^{e}, and —R^{f};

The variables as used in the description of optional substituents for lower alkyl, lower alkenyl, lower alkynyl, cycloalkyl, heterocycloalkyl, aryl and heteroaryl are defined as follows:
—R^{a}, —R^{b}, and —R^{c} at each occurrence are independently selected from the group consisting of hydrogen, —R^{d}, —R^{e}, and —R^{f}, provided, however, that R^{a} bound to S, S(O), S(O)₂, C(S) or C(O) is not hydrogen, or
—R^{b} and —R^{c} combine with the nitrogen to which they are attached form a 5-7 membered heterocycloalkyl or a 5 or 7 membered nitrogen containing heteroaryl, wherein the 5-7 membered heterocycloalkyl or 5 or 7 membered nitrogen containing heteroaryl are optionally substituted with one or more, preferably 1, 2, 3, 4 or 5, also 1, 2, or 3 substituents selected from the group consisting of halogen, cycloalkylamino, —NO₂, —CN, —OR^{k}, —SR^{k}, —NR^{k}R^{k}, —R^{m}, and —R^{o};
—R^{d} at each occurrence is independently lower alkyl optionally substituted with one or more, preferably 1, 2, 3, 4 or 5, also 1, 2 or 3 substituents selected from the group consisting of fluoro, —OR^{g}, —SR^{g}, —NR^{g}R^{g}, —C(O)R^{g}, —C(S)R^{g}, —S(O)R^{g}, —S(O)₂R^{g}, —C(O)NR^{g}R^{g}, —C(S)NR^{g}R^{g}, —S(O)₂NR^{g}R^{g}, —NR^{g}C(O)R^{g}, —NR^{g}C(S)R^{g}, —NR^{g}S(O)₂R^{g}, —NR^{g}C(O)NR^{g}R^{g}, —NR^{g}C(S)NR^{g}R^{g}, —NR^{g}S(O)₂NR^{g}R^{g}, and —R^{f};
—R^{e} at each occurrence is independently selected from the group consisting of lower alkenyl and lower alkynyl, wherein lower alkenyl or lower alkynyl are optionally substituted with one or more, preferably 1, 2, 3, 4 or 5, also 1, 2 or 3 substituents selected from the group consisting of fluoro, —OR^{g}, —SR^{g}, —NR^{g}R^{g}, —C(O)R^{g}, —C(S)R^{g}, —S(O)R^{g}, —S(O)₂R^{g}, —C(O)NR^{g}R^{g}, —C(S)NR^{g}R^{g}, —S(O)₂NR^{g}R^{g}, —NR^{g}C(O)R^{g}, —NR^{g}C(S)R^{g}, —NR^{g}S(O)₂R^{g}, —NR^{g}C(O)NR^{g}R^{g}, —NR^{g}C(S)NR^{g}R^{g}, —NR^{g}S(O)₂NR^{g}R^{g}, —R^{d}, and —R^{f};
—R^{f} at each occurrence is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2 or 3 substituents selected from the group consisting of halogen, —NO₂, —CN, —OR^{g}, —SR^{g}, —NR^{g}R^{g}, —C(O)R^{g}, —C(S)R^{g}, —S(O)R^{g}, —S(O)₂R^{g}, —C(O)NR^{g}R^{g}, —C(S)NR^{g}R^{g}, —S(O)₂NR^{g}R^{g}, —NR^{g}C(O)R^{g}, —NR^{g}C(S)R^{g}, —NR^{g}S(O)₂R^{g}, —NR^{g}C(O)NR^{g}R^{g}, —NR^{g}C(S)NR^{g}R^{g}, —NR^{g}S(O)₂NR^{g}R^{g}, —R^{m}, and —R^{o};
—R^{g} at each occurrence is independently selected from the group consisting of hydrogen, —R^{h}, —Rⁱ, and —R^{j}, provided, however, that R^{g} bound to S, S(O), S(O)₂, C(S) or C(O) is not hydrogen;
—R^{h} at each occurrence is independently lower alkyl optionally substituted with one or more, preferably 1, 2, 3, 4 or 5, also 1, 2, or 3 substituents selected from the group consisting of fluoro, —OR^{k}, —SR^{k}, —NR^{k}R^{k}, —C(O)R^{k}, —C(S)R^{k}, —S(O)R^{k}, —S(O)₂R^{k}, —C(O)NR^{k}R^{k}, —C(S)NR^{k}R^{k}, —S(O)₂NR^{k}R^{k}, —NR^{k}C(O)R^{k}, —NR^{k}C(S)R^{k}, —NR^{k}S(O)₂R^{k}, —NR^{k}C(O)NR^{k}R^{k}, —NR^{k}C(S)NR^{k}R^{k}, —NR^{k}S(O)₂NR^{k}R^{k}, and —R^{o}, provided, however, that any substitution on the alkyl carbon bound to any O, S, or N of any OR^{h}, SR^{h}, or NR^{h} is selected from the group consisting of fluoro and —R^{o};
—Rⁱ at each occurrence is independently selected from the group consisting of C₃₋₆ alkenyl and C₃₋₆ alkynyl, wherein C₃₋₆ alkenyl or C₃₋₆ alkynyl are optionally substituted with one or more, preferably 1, 2, 3, 4 or 5, also 1, 2, or 3 substituents selected from the group consisting of fluoro, —OR^{k}, —SR^{k}, —NR^{k}R^{k}, —C(O)R^{k}, —C(S)R^{k}, —S(O)R^{k}, —S(O)₂R^{k}, —C(O)NR^{k}R^{k}, —C(S)NR^{k}R^{k}, S(O)₂NR^{k}R^{k}, —NR^{k}C(O)R^{k}, —NR^{k}C(S)R^{k}, —NR^{k}S(O)₂R^{k}, —NR^{k}C(O)NR^{k}R^{k}, —NR^{k}C(S)NR^{k}R^{k}, —NR^{k}S(O)₂NR^{k}R^{k}, —R^{m} and —R^{o}, provided, however, that any substitution on the alkenyl or alkynyl carbon bound to any O, S, or N of any ORⁱ, SRⁱ, or NRⁱ is selected from the group consisting of fluoro, —R^{m} and —R^{o};
R^{j} at each occurrence is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2, or 3 substituents selected from the group consisting of halogen, —NO₂, —CN, —OR^{k}, —SR^{k}, —NR^{k}R^{k}, C(O)R^{k}, —C(S)R^{k}, —S(O)R^{k}, —S(O)₂R^{k}, —C(O)NR^{k}R^{k}, —C(S)NR^{k}R^{k}, —S(O)₂NR^{k}R^{k}, —NR^{k}C(O)R^{k}, —NR^{k}C(S)R^{k}, —NR^{k}S(O)₂R^{k}, —NR^{k}C(O)NR^{k}R^{k}, —NR^{k}C(S)NR^{k}R^{k}, —NR^{k}S(O)₂NR^{k}R^{k}, —R^{m}, and —R^{o};
—R^{m} at each occurrence is independently selected from the group consisting of lower alkyl, lower alkenyl and lower alkynyl, wherein lower alkyl is optionally substituted with one or more, preferably 1, 2, 3, 4 or 5, also 1, 2, or 3 substituents selected from the group consisting of —R^{o}, fluoro, lower alkoxy, fluoro substituted lower alkoxy, lower alkylthio, fluoro substituted lower alkylthio, mono-alkylamino, di-alkylamino, and cycloalkylamino, and wherein lower alkenyl or lower alkynyl are optionally substituted with one or more, preferably 1, 2, 3, 4 or 5, also 1, 2, or 3 substituents selected from the group consisting of —R^{o}, fluoro, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
—R^{k} at each occurrence is independently selected from the group consisting of hydrogen, —Rⁿ, and —R^{o}, provided, however, that R^{k} bound to S, S(O), S(O)₂, C(S) or C(O) is not hydrogen;
—Rⁿ at each occurrence is independently selected from the group consisting of lower alkyl, C₃₋₆ alkenyl and C₃₋₆ alkynyl, wherein lower alkyl is optionally substituted with one or more, preferably 1, 2, 3, 4 or 5, also 1, 2, or 3 substituents selected from the group consisting of —R^{o}, fluoro, lower alkoxy, fluoro substituted lower alkoxy, lower alkylthio, fluoro substituted lower alkylthio, mono-alkylamino, di-alkylamino, and cycloalkylamino, provided, however, that any substitution of the lower alkyl carbon bound to the O of ORⁿ, S of SRⁿ, or N of any NRⁿ is fluoro or —R^{o}, and wherein C₃₋₆ alkenyl or C₃₋₆ alkynyl are optionally substituted with one or more, preferably 1, 2, 3, 4 or 5, also 1, 2, or 3 substituents selected from the group consisting of —R^{o}, fluoro, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino, provided, however, that any substitution of the C₃₋₆ alkenyl or C₃₋₆ alkynyl carbon bound to the the O of ORⁿ, S of SRⁿ, or N of any NRⁿ is fluoro, lower alkyl, fluoro substituted lower alkyl, or —R^{o};
—R^{o} at each occurrence is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2, or 3 substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino.

“Lower alkoxy” denotes the group —OR^{p}, where R^{P} is lower alkyl. “Optionally substituted lower alkoxy” denotes lower alkoxy in which R^{P} is optionally substituted lower alkyl. Preferably, substitution of lower alkoxy is with 1, 2, 3, 4, or 5 substituents, also 1, 2, or 3 substituents. For example “fluoro substituted lower alkoxy” denotes lower alkoxy in which the lower alkyl is substituted with one or more fluoro atoms, where preferably the lower alkoxy is substituted with 1, 2, 3, 4 or 5 fluoro atoms, also 1, 2, or 3 fluoro atoms. It is understood that substitutions on lower alkoxy are attached at any available atom to produce a stable compound, substitution of lower alkoxy is such that O, S, or N (except where N is a heteroaryl ring atom), are not bound to the alkyl carbon bound to the O of lower alkoxy. Further, where lower alkoxy is described as a substituent of another moiety, the O of lower alkoxy is not bound to a carbon atom that is bound to an O, S, or N of the other moiety (except where N is a heteroaryl ring atom), or to an alkene or alkyne carbon of the other moiety.

“Aryloxy” denotes the group —OR^{q}, where R^{q} is aryl. “Optionally substituted aryloxy” denotes aryloxy in which R^{q} is optionally substituted aryl. “Heteroaryloxy” denotes the group —OR^{r}, where R^{r} is heteroaryl. “Optionally substituted heteroaryloxy” denotes heteroaryloxy in which R^{r} is optionally substituted heteroaryl.

“Lower alkylthio” denotes the group —SR^{s}, where R^{s} is lower alkyl. “Substituted lower alkylthio” denotes lower alkylthio in which R^{s} is optionally substituted lower alkyl. Preferably, substitution of lower alkylthio is with 1, 2, 3, 4, or 5 substituents, also 1, 2, or 3 substituents. For example “fluoro substituted lower alkylthio” denotes lower alkylthio in which the lower alkyl is substituted with one or more fluoro atoms, where preferably the lower alkylthio is substituted with 1, 2, 3, 4 or 5 fluoro atoms, also 1, 2, or 3 fluoro atoms. It is understood that substitutions on lower alkylthio are attached at any available atom to produce a stable compound, substitution of lower alkylthio is such that O, S, or N (except where N is a heteroaryl ring atom), are not bound to the alkyl carbon bound to the S of lower alkylthio. Further, where lower alkylthio is described as a substituent of another moiety, the lower alkylthio S is not bound to a carbon atom that is bound to an O, S, or N of the other moiety (except where N is a heteroaryl ring atom), or to an alkene or alkyne carbon of the other moiety.

“Amino” or “amine” denotes the group —NH₂. “Mono-alkylamino” denotes the group —NHR^{t} where R^{t} is lower alkyl. “Di-alkylamino” denotes the group —NR^{t}R^{u}, where R^{t} and R^{u} are independently lower alkyl. “Cycloalkylamino” denotes the group —NR^{v}R^{w}, where R^{v} and R^{w} combine with the nitrogen to form a 5-7 membered heterocycloalkyl, where the heterocycloalkyl may contain an additional heteroatom within the ring, such as O, N, or S, and may also be further substituted with lower alkyl. Examples of cycloalkylamino include, but are not limited to, piperidine, piperazine, 4-methylpiperazine, morpholine, and thiomorpholine. It is understood that when mono-alkylamino, di-alkylamino, or cycloalkylamino are substituents on other moieties that are attached at any available atom to produce a stable compound, the nitrogen of mono-alkylamino, di-alkylamino, or cycloalkylamino as substituents is not bound to a carbon atom that is bound to an O, S, or N of the other moiety (except where N is a heteroaryl ring atom) or to an alkene or alkyne carbon of the other moiety.

As used herein in connection with PPAR modulating compound, binding compounds or ligands, the term “specific for PPAR” and terms of like import mean that a particular compound binds to a PPAR to a statistically greater extent than to other biomolecules that may be present in or originally isolated from a particular organism, e.g., at least 2, 3, 4, 5, 10, 20, 50, 100, or 1000-fold greater binding. Also, where biological activity other than binding is indicated, the term “specific for PPAR” indicates that a particular compound has greater biological activity associated with binding to a PPAR than to other biomolecules (e.g., at a level as indicated for binding specificity). Similarly, the specificity can be for a specific PPAR with respect to other PPARs that may be present in or originally isolated from a particular organism.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. In some cases, the specificity is with reference to a limited set of other biomolecules, e.g., in the case of PPARs, in some cases the reference may be other receptors, or for a particular PPAR, it may be other PPARs. In some embodiments, the greater specificity is at least 2, 3, 4, 5, 8, 10, 50, 100, 200, 400, 500, or 1000-fold greater specificity. In the context of ligands interacting with PPARs, the terms “activity on”, “activity toward,” and like terms mean that such ligands have IC₅₀ EC₅₀ less than 10 μM, less than 1 μM, less than 100 nM, less than 50 nM, less than 20 nM, less than 10 nM, less than 5 nM, or less than 1 nM with respect to at least one PPAR as determined in a generally accepted PPAR activity assay.

The term “composition” or “pharmaceutical composition” refers to a formulation suitable for administration to an intended animal subject for therapeutic purposes. The formulation includes a therapeutically significant quantity (i.e. a therapeutically effective amount) of at least one active compound and at least one pharmaceutically acceptable carrier or excipient, which is prepared in a form adapted for administration to a subject. Thus, the preparation is “pharmaceutically acceptable”, indicating that it does not have properties that would cause a reasonably prudent medical practitioner to avoid administration of the material to a patient, taking into consideration the disease or conditions to be treated and the respective route of administration. In many cases, such a pharmaceutical composition is a sterile preparation, e.g. for injectibles.

The term “PPAR-mediated” disease or condition and like terms refer to a disease or condition in which the biological function of a PPAR affects the development and/or course of the disease or condition, and/or in which modulation of PPAR alters the development, course, and/or symptoms of the disease or condition. Similarly, the phrase “PPAR modulation provides a therapeutic benefit” indicates that modulation of the level of activity of PPAR in a subject indicates that such modulation reduces the severity and/or duration of the disease, reduces the likelihood or delays the onset of the disease or condition, and/or causes an improvement in one or more symptoms of the disease or condition. In some cases the disease or condition may be mediated by any one or more of the PPAR isoforms, e.g., PPARγ, PPARα, PPARδ, PPARγ and PPARα, PPARγ and PPARδ, PPARα and PPARδ, or PPARγ, PPARα, and PPARδ.

The term “therapeutically effective” or “effective amount” indicates that the materials or amount of material is effective to prevent, alleviate, or ameliorate one or more symptoms of a disease or medical condition, and/or to prolong the survival of the subject being treated.

The term “PPAR” refers to a peroxisome proliferator-activated receptor as recognized in the art. As indicated above, the PPAR family includes PPARα (also referred to as PPARa or PPARalpha), PPARδ (also referred to as PPARd or PPARdelta), and PPARγ (also referred to as PPARg or PPARgamma). The individual PPARs can be identified by their sequences, where exemplary reference sequence accession numbers are as follows:

| Receptor | Sequence | Accession No. | SEQ ID NO: |
|---|---|---|---|
| hPPARa | cDNA | NM_005036 | |
| hPPARa | protein | NP_005027 | |
| hPPARg isoform 2 | cDNA | NM_015869 | |
| hPPARg isoform 2 | protein | NP_056953 | |
| hPPARd | cDNA | NM_006238 | |
| hPPARd | protein | NP_006229 | |

One of ordinary skill in the art will recognize that sequence differences will exist due to allelic variation, and will also recognize that other animals, particularly other mammals have corresponding PPARs, which have been identified or can be readily identified using sequence alignment and confirmation of activity. Such homologous PPARs can also be used in the present invention, which homologous PPARs have sequence identity of, for example, at least 50%, 60%, 70%, 80%, 90%, 95%, 99%, or even 100%, over a region spanning 50, 100, 150, 200, 250, 300, 350, 400, 450, 500, or even more amino acids or nucleotides for proteins or nucleic acids, respectively. One of ordinary skill in the art will also recognize that modifications can be introduced in a PPAR sequence without destroying PPAR activity. Such modified PPARs can also be used in the present invention, e.g., if the modifications do not alter the binding site conformation to the extent that the modified PPAR lacks substantially normal ligand binding.

As used herein in connection with the design or development of ligands, the term “bind” and “binding” and like terms refer to a non-convalent energetically favorable association between the specified molecules (i.e., the bound state has a lower free energy than the separated state, which can be measured calorimetrically). For binding to a target, the binding is at least selective, that is, the compound binds preferentially to a particular target or to members of a target family at a binding site, as compared to non-specific binding to unrelated proteins not having a similar binding site. For example, BSA is often used for evaluating or controlling for non-specific binding. In addition, for an association to be regarded as binding, the decrease in free energy going from a separated state to the bound state must be sufficient so that the association is detectable in a biochemical assay suitable for the molecules involved.

By “assaying” is meant the creation of experimental conditions and the gathering of data regarding a particular result of the experimental conditions. For example, enzymes can be assayed based on their ability to act upon a detectable substrate. Likewise, for example, a compound or ligand can be assayed based on its ability to bind to a particular target molecule or molecules and/or to modulate an activity of a target molecule.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “clog P” is meant the calculated log P of a compound, “P” referring to the partition coefficient of the compound between a lipophilic and an aqueous phase, usually between octanol and water.

In the context of compounds binding to a target, the term “greater affinity” indicates that the compound binds more tightly than a reference compound, or than the same compound in a reference condition, i.e., with a lower dissociation constant. In some embodiments, the greater affinity is at least 2, 3, 4, 5, 8, 10, 50, 100, 200, 400, 500, 1000, or 10,000-fold greater affinity.

By binding with “moderate affinity” is meant binding with a K_{D} of from about 200 nM to about 1 μM under standard conditions. By “moderately high affinity” is meant binding at a K_{D} of from about 1 nM to about 200 nM. By binding at “high affinity” is meant binding at a K_{D} of below about 1 nM under standard conditions. The standard conditions for binding are at pH 7.2 at 37° C. for one hour. For example, typical binding conditions in a volume of 100 μl/well would comprise a PPAR, a test compound, HEPES 50 mM buffer at pH 7.2, NaCl 15 mM, ATP 2 μM, and bovine serum albumin (1 ug/well), at 37° C. for one hour.

Binding compounds can also be characterized by their effect on the activity of the target molecule. Thus, a “low activity” compound has an inhibitory concentration (IC₅₀) (for inhibitors or antagonists) or effective concentration (EC₅₀) (applicable to agonists) of greater than 1 μM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured. For PPAR agonists, activities can be determined as described in the Examples, or using other such assay methods known in the art.

By “protein” is meant a polymer of amino acids. The amino acids can be naturally or non-naturally occurring. Proteins can also contain modifications, such as being glycosylated, phosphorylated, or other common modifications.

By “protein family” is meant a classification of proteins based on structural and/or functional similarities. For example, kinases, phosphatases, proteases, and similar groupings of proteins are protein families. Proteins can be grouped into a protein family based on having one or more protein folds in common, a substantial similarity in shape among folds of the proteins, homology, or based on having a common function. In many cases, smaller families will be specified, e.g., the PPAR family.

By “specific biochemical effect” is meant a therapeutically significant biochemical change in a biological system causing a detectable result. This specific biochemical effect can be, for example, the inhibition or activation of an enzyme, the inhibition or activation of a protein that binds to a desired target, or similar types of changes in the body's biochemistry. The specific biochemical effect can cause alleviation of symptoms of a disease or condition or another desirable effect. The detectable result can also be detected through an intermediate step.

By “standard conditions” is meant conditions under which an assay is performed to obtain scientifically meaningful data. Standard conditions are dependent on the particular assay, and can be generally subjective. Normally the standard conditions of an assay will be those conditions that are optimal for obtaining useful data from the particular assay. The standard conditions will generally minimize background signal and maximize the signal sought to be detected.

By “standard deviation” is meant the square root of the variance. The variance is a measure of how spread out a distribution is. It is computed as the average squared deviation of each number from its mean. For example, for the numbers 1, 2, and 3, the mean is 2 and the variance is

$${σ}^{2} = \frac{{( 1 - 2 )}^{2} + {( 2 - 2 )}^{2} + {( 3 - 2 )}^{2}}{3} = 0.667 .$$

In the context of this invention, by “target molecule” is meant a molecule that a compound, molecular scaffold, or ligand is being assayed for binding to. The target molecule has an activity that binding of the molecular scaffold or ligand to the target molecule will alter or change. The binding of the compound, scaffold, or ligand to the target molecule can preferably cause a specific biochemical effect when it occurs in a biological system. A “biological system” includes, but is not limited to, a living system such as a human, animal, plant, or insect. In most but not all cases, the target molecule will be a protein or nucleic acid molecule.

By “pharmacophore” is meant a representation of molecular features that are considered to be responsible for a desired activity, such as interacting or binding with a receptor. A pharmacophore can include 3-dimensional (hydrophobic groups, charged/ionizable groups, hydrogen bond donors/acceptors), 2D (substructures), and 1D (physical or biological) properties.

As used herein in connection with numerical values, the terms “approximately” and “about” ±mean 10% of the indicated value.

### I. Applications of PPAR Agonists

The PPARs have been recognized as suitable targets for a number of different diseases and conditions. Some of those applications are described briefly below. Additional applications are known and the present compounds can also be used for those diseases and conditions.

(a) Insulin resistance and diabetes: In connection with insulin resistance and diabetes, PPARγ is necessary and sufficient for the differentiation of adipocytes in vitro and in vivo. In adipocytes, PPARγ increases the expression of numerous genes involved in lipid metabolism and lipid uptake. In contrast, PPARγ down-regulates leptin, a secreted, adipocyte-selective protein that has been shown to inhibit feeding and augment catabolic lipid metabolism. This receptor activity could explain the increased caloric uptake and storage noted in vivo upon treatment with PPAR-γ agonists. Clinically, TZDs, including troglitazone, rosiglitazone, and pioglitazone, and non-TZDs, including farglitazar, have insulin-sensitizing and antidiabetic activity. (Berger et al., 2002, Diabetes Tech. And Ther. 4:163-174.)

PPARγ has been associated with several genes that affect insulin action. TNFα, a proinflammatory cytokine that is expressed by adipocytes, has been associated with insulin resistance. PPAR-γ agonists inhibit expression of TNFα in adipose tissue of obese rodents, and ablate the actions of TNFα in adipocytes in vitro. PPARγ agonists were shown to inhibit expression of 10-hydroxysteroid dehydrogenase 1 (11β-HSD-1), the enzyme that converts cortisone to the glucocorticoid agonist cortisol, in adipocytes and adipose tissue of type 2 diabetes mouse models. This is noteworthy since hypercortico-steroidism exacerbates insulin resistance. Adipocyte Complement-Related Protein of 30 kDa (Acrp30 or adiponectin) is a secreted adipocyte-specific protein that decreases glucose, triglycerides, and free fatty acids. In comparison to normal human subjects, patients with type 2 diabetes have reduced plasma levels of Acrp30. Treatment of diabetic mice and nondiabetic human subjects with PPARγ agonists increases plasma levels of Acrp30. Induction of Acrp30 by PPARγ agonists might therefore also play a key role in the insulin-sensitizing mechanism of PPARγ agonists in diabetes. (Berger et al., supra).

PPARγ is expressed predominantly in adipose tissue. Thus, it is believed that the net in vivo efficacy of PPAR-γ agonists involves direct actions on adipose cells with secondary effects in key insulin responsive tissues such as skeletal muscle and liver. This is supported by the lack of glucose-lowering efficacy of rosiglitazone in a mouse model of severe insulin resistance where white adipose tissue was essentially absent. Furthermore, in vivo treatment of insulin resistant rats produces acute (<24 h) normalization of adipose tissue insulin action whereas insulin-mediated glucose uptake in muscle was not improved until several days after the initiation of therapy. This is consistent with the fact that PPARγ agonists can produce an increase in adipose tissue insulin action after direct in vitro incubation, whereas no such effect could be demonstrated using isolated in vitro incubated skeletal muscles. The beneficial metabolic effects of PPARγ agonists on muscle and liver may be mediated by their ability to (a) enhance insulin-mediated adipose tissue uptake, storage (and potentially catabolism) of free fatty acids; (b) induce the production of adipose-derived factors with potential insulin sensitizing activity (e.g., Acrp30); and/or (c) suppress the circulating levels and/or actions of insulin resistance-causing adipose-derived factors such as TNFα or resistin. (Berger et al., supra).

(b) Dyslipidemia and atherosclerosis: In connection with dyslipidemia and atherosclerosis, PPARα has been shown to play a critical role in the regulation of cellular uptake, activation, and β-oxidation of fatty acids. Activation of PPARα induces expression of fatty acid transport proteins and enzymes in the peroxisomal β-oxidation pathway. Several mitochondrial enzymes involved in the energy-harvesting catabolism of fatty acids are robustly upregulated by PPARα agonists. Peroxisome proliferators also activate expression of the CYP4As, a subclass of cytochrome P450 enzymes that catalyze the ω-hydroxylation of fatty acids, a pathway that is particularly active in the fasted and diabetic states. In sum, it is clear that PPARα is an important lipid sensor and regulator of cellular energy-harvesting metabolism. (Berger et al., supra).

Atherosclerosis is a very prevalent disease in Westernized societies. In addition to a strong association with elevated LDL cholesterol, “dyslipidemia” characterized by elevated triglyceride-rich particles and low levels of HDL cholesterol is commonly associated with other aspects of a metabolic syndrome that includes obesity, insulin resistance, type 2 diabetes, and an increased risk of coronary artery disease. Thus, in 8,500 men with known coronary artery disease, 38% were found to have low HDL (<35 mg/dL) and 33% had elevated triglycerides (>200 mg/dL). In such patients, treatment with fibrates resulted in substantial triglyceride lowering and modest HDL-raising efficacy. More importantly, a recent large prospective trial showed that treatment with gemfibrozil produced a 22% reduction in cardiovascular events or death. Thus PPARα agonists can effectively improve cardiovascular risk factors and have a net benefit to improve cardiovascular outcomes. In fact, fenofibrate was recently approved in the United States for treatment of type IIA and IIB hyper-lipidemia. Mechanisms by which PPARα activation cause triglyceride lowering are likely to include the effects of agonists to suppress hepatic apo-CIII gene expression while also stimulating lipoprotein lipase gene expression. Dual PPARγ/α agonists, including KRP-297 and DRF 2725, possess potent lipid-altering efficacy in addition to antihyperglycemic activity in animal models of diabetes and lipid disorders.

The presence of PPARα and/or PPARγ expression in vascular cell types, including macrophages, endothelial cells, and vascular smooth muscle cells, suggests that direct vascular effects might contribute to potential antiatherosclerosis efficacy. PPARα and PPARα activation have been shown to inhibit cytokine-induced vascular cell adhesion and to suppress monocyte-macrophage migration. Several additional studies have also shown that PPARγ-selective compounds have the capacity to reduce arterial lesion size and attenuate monocyte-macrophage homing to arterial lesions in animal models of atherosclerosis. PPARγ is present in macrophages in human atherosclerotic lesions, and may play a role in regulation of expression of matrix metalloproteinase-9 (MMP-9), which is implicated in atherosclerotic plaque rupture (Marx et al., Am J Pathol. 1998, 153(1):17-23). Downregulation of LPS induced secretion of MMP-9 was also observed for both PPARα and PPARγ agonists, which may account for beneficial effects observed with PPAR agonists in animal models of atherosclerosis (Shu et al., Biochem Biophys Res Commun. 2000, 267(1):345-9). PPARγ is also shown to have a role in intercellular adhesion molecule-1 (ICAM-1) protein expression (Chen et al., Biochem Biophys Res Commun. 2001, 282(3):717-22) and vascular cell adhesion molecule-1 (VCAM-1) protein expression (Jackson et al., Arterioscler Thromb Vasc Biol. 1999, 19(9):2094-104) in endothelial cells, both of which play a role in the adhesion of monocytes to endothelial cells. In addition, two recent studies have suggested that either PPARα or PPARγ activation in macrophages can induce the expression of a cholesterol efflux “pump” protein.

It has been found that relatively selective PPARδ agonists produce minimal, if any, glucose- or triglyceride-lowering activity in murine models of type 2 diabetes in comparison with efficacious PPARγ or PPARα agonists. Subsequently, a modest increase in HDL-cholesterol levels was detected with PPARδ agonists in db/db mice. Recently, Oliver et al. (supra) reported that a potent, selective PPARδ agonist could induce a substantial increase in HDL-cholesterol levels while reducing triglyceride levels and insulin resistance in obese rhesus monkeys.

Thus, via multifactorial mechanisms that include improvements in circulating lipids, systemic and local antiinflammatory effects, and, inhibition of vascular cell proliferation, PPARα, PPARγ, and PPARδ agonists can be used in the treatment or prevention of atherosclerosis (Berger et al., supra).

(c) Inflammation: Monocytes and macrophages are known to play an important part in the inflammatory process through the release of inflammatory cytokines and the production of nitric oxide by inducible nitric oxide synthase. Rosiglitazone has been shown to induce apoptosis of macrophages at concentrations that parallel its affinity for PPARγ. This ligand has also been shown to block inflammatory cytokine synthesis in colonic cell lines. This latter observation suggests a mechanistic explanation for the observed anti-inflammatory actions of TZDs in rodent models of colitis.

Additional studies have examined the relationship between macrophages, cytokines and PPARγ and agonists thereof (Jiang et al., Nature 1998, 391(6662):82-6., Ricote et al., Nature 1998, 391(6662):79-82, Hortelano et al., J Immunol. 2000, 165(11):6525-31, and Chawla et al., Nat. Med. 2001, 7(1):48-52) suggesting a role for PPARγ agonists in treating inflammatory responses, for example in autoimmune diseases.

The migration of monocytes and macrophages plays a role in the development of inflammatory responses as well. PPAR ligands have been shown to have an effect on a variety of chemokines. Monocyte chemotactic protein-1 (MCP-1) directed migration of monocytes is attenuated by PPARγ and PPARα ligands in a monocytic leukemia cell line (Kintscher et al., Eur J Pharmacol. 2000, 401(3):259-70). MCP-1 gene expression was shown to be suppressed by PPARγ ligand 15-deoxy-Delta(12,14)PGJ2 (15d-PGJ2) in two monocytic cell lines, which also showed induction of IL-8 gene expression (Zhang et al., J Immunol. 2001, 166(12):7104-11).

Anti-inflammatory actions have been described for PPARα ligands that can be important in the maintenance of vascular health. Treatment of cytokine-activated human macrophages with PPARα agonists induced apoptosis of the cells. It was reported that PPARα agonists inhibit activation of aortic smooth muscle cells in response to inflammatory stimuli. (Staels et al., 1998, Nature 393:790-793.) In hyperlipidemic patients, fenofibrate treatment decreases the plasma concentrations of the inflammatory cytokine interleukin-6.

Anti-inflammatory pathways in airway smooth muscle cells were investigated with respect to PPARα and PPARγ (Patel et al., 2003, The Journal of Immunology, 170:2663-2669). This study demonstrated and anti-inflammatory effect of a PPARγ ligand that may be useful in the treatment of COPD and steroid-insensitive asthma.

The anti-inflammatory effects of PPAR modulators have also been studied with respect to autoimmune diseases, such as chronic inflammatory bowel syndrome, arthritis, Crohn's disease and multiple sclerosis, and in neuronal diseases such as Alzheimer's disease and Parkinson's disease.

(d) Hypertension: Hypertension is a complex disorder of the cardiovascular system that has been shown to be associated with insulin resistance. Type 2 diabetes patients demonstrate a 1.5-2-fold increase in hypertension in comparison with the general population. Troglitazone, rosiglitazone, and pioglitazone therapy have been shown to decrease blood pressure in diabetic patients as well as troglitazone therapy in obese, insulin-resistant subjects. Since such reductions in blood pressure were shown to correlate with decreases in insulin levels, they can be mediated by an improvement in insulin sensitivity. However, since TZDs also lowered blood pressure in one-kidney one-clip Sprague Dawley rats, which are not insulin resistant, it was proposed that the hypotensive action of PPARγ agonists is not exerted solely through their ability to improve insulin sensitivity. Other mechanisms that have been invoked to explain the antihypertensive effects of PPARγ agonists include their ability to (a) downregulate expression of peptides that control vascular tone such as PAI-I, endothelin, and type-c natriuretic peptide C or (b) alter calcium concentrations and the calcium sensitivity of vascular cells (Berger et al., supra).

(e) Cancer: PPAR modulation has also been correlated with cancer treatment. (Burstein et al.; Breast Cancer Res. Treat. 2003, 79(3):391-7; Alderd et al., Oncogene, 2003, 22(22):3412-6).

(f) Weight Control: Administration of PPARα agonists can induce satiety, and thus are useful in weight loss or maintenance. Such PPARα agonists can act preferentially on PPARα, or can also act on another PPAR, or can be PPAR pan-agonists. Thus, the satiety inducing effect of PPARα agonists can be used for weight control or loss.

(g) Autoimmune diseases: PPAR agonists may provide benefits in the treatment of autoimmune diseases. Agonists of PPAR isoforms may be involved in T cell and B cell trafficking or activity, the altering of oligodendrocyte function or differentiation, the inhibition of macrophage activity, the reduction of inflammatory responses, and neuroprotective effects, some or all of which may be important in a variety of autoimmune diseases.

Multiple sclerosis (MS) is a neurodegenerative autoimmune disease that involves the demyelination of axons and formation of plaques. PPARδ mRNA has been shown to be strongly expressed in immature oligodendrocytes (Granneman et al., J Neurosci Res. 1998, 51(5):563-73). PPARδ selective agonists or pan-agonists were shown to accelerate differentiation of oligodendrocytes, with no effect on differentiation observed with a PPARγ selective agonist. An alteration in the myelination of corpus callosum was observed in PPARδ null mice (Peters et al., Mol Cell Biol. 2000, 20(14):5119-28). It was also shown that PPARδ mRNA and protein is expressed throughout the brain in neurons and oligodendrocytes, but not in astrocytes (Woods et al., Brain Res. 2003, 975(1-2):10-21). These observations suggest that PPARδ has a role in myelination, where modulation of such a role could be used to treat multiple sclerosis by altering the differentiation of oligodendrocytes, which may result in slowing of the demyelination, or even promoting the remyelination of axons. It has also been shown that oligodendrocyte-like B12 cells, as well as isolated spinal cord oligodendrocytes from rat, are affected by PPARγ agonists. Alkyl-dihydroxyacetone phosphate synthase, a key peroxisomal enzyme involved in the synthesis of plasmologens, which are a key component of myelin, is increased in PPARγ agonist treated B12 cells, while the number of mature cells in isolated spinal cord oligodendrocytes increases with PPARγ agonist treatment.

The role of PPAR in the regulation of B and T cells may also provide therapeutic benefits in diseases such as MS. For example, it has been shown that PPARγ agonists can inhibit the secretion of IL-2 by T cells (Clark et al., J Immunol. 2000, 164(3):1364-71) or may induce apoptosis in T cells (Harris et al., Eur J Immunol. 2001, 31(4):1098-105), suggesting an important role in cell-mediated immune responses. An antiproliferative and cytotoxic effect on B cells by PPARγ agonists has also been observed (Padilla et al., Clin Immunol. 2002, 103(1):22-33).

The anti-inflammatory effects of PPAR modulators, as discussed herein, may also be useful in treating MS, as well as a variety of other autoimmune diseases such as Type-1 diabetes mellitus, psoriasis, vitiligo, uveitis, Sjogren's disease, pemphigus foliaceus, inclusion body myositis, polymyositis, dermatomyositis, scleroderma, Grave's disease, Hashimoto's disease, chronic graft-versus host disease, rheumatoid arthritis, inflammatory bowel syndrome, and Crohn's disease. Using a mouse model, the PPARα agonists gemfibrozil and fenofibrate were shown to inhibit clinical signs of experimental autoimmune encephalomyelitis, suggesting that PPARα agonists may be useful in treating inflammatory conditions such as multiple sclerosis (Lovett-Racke et al., J Immunol. 2004, 172(9):5790-8).

Neuroprotective effects that appear to be associated with PPARs may also aid in the treatment of MS. The effects of PPAR agonists on LPS induced neuronal cell death were studied using cortical neuron-glial co-cultures. PPARγ agonists 15d-PGJ2, ciglitazone and troglitazone were shown to prevent the LPS-induced neuronal cell death, as well as abolish NO and PGE2 release and a reduction in iNOS and COX-2 expression (Kim et al., Brain Res. 2002, 941(1-2): 1-10).

Rheumatoid arthritis (RA) is an autoimmune inflammatory disease that results in the destruction of joints. In addition to chronic inflammation and joint damage due in part to mediators such as IL-6 and TNF-alpha, osteoclast differentiation is also implicated in damage to the joints. PPAR agonists may regulate these pathways, providing therapeutic benefits in treatment of RA. In studies using PPARγ agonist troglitazone in fibroblast-like synovial cells (FLS) isolated from patients with rheumatoid arthritis, an inhibition of cytokine mediated inflammatory responses was observed (Yamasaki et al., Clin Exp Immunol., 2002, 129(2):379-84). PPARγ agonists have also demonstrated beneficial effects in a rat or mouse model of RA (Kawahito et al., J Clin Invest. 2000, 106(2): 189-97; Cuzzocrea et al., Arthritis Rheum. 2003, 48(12):3544-56). The effects of the PPARα ligand fenofibrate on rheumatoid synovial fibroblasts from RA patients also showed inhibition of cytokine production, as well as NF-KappaB activation and osteoclast differentiation. Fenofibrate was also shown to inhibit the development of arthritis in a rat model (Okamoto et al., Clin Exp Rheumatol. 2005, 23(3):323-30).

Psoriasis is a T cell mediated autoimmune disease, where T cell activation leads to release of cytokines and resulting proliferation of keratinocytes. In addition to anti-inflammatory effects, the differentiation of keratinocytes may also be a therapeutic target for PPAR agonists. Studies in a PPARδ null mouse model suggest using PPARδ ligand to selectively induce keratinocyte differentiation and inhibit cell proliferation (Kim et al., Cell Death Differ. 2005). Thiazolidinedione ligands of PPARγ have been shown to inhibit the proliferation of psoriatic keratinocytes in monolayer and organ culture, and when applied topically inhibit epidermal hyperplasia of human psoriatic skin transplanted to SCID mice (Bhagavathula et al., J Pharmacol Exp Ther. 2005, 315(3):996-1004).

(h) Neurodegenerative diseases: The modulation of the PPARs may provide benefits in the treatment of neuronal diseases. For example, the anti-inflammatory effects of PPAR modulators discussed herein have also been studied with respect to neuronal diseases such as Alzheimer's disease and Parkinson's disease.

In addition to inflammatory processes, Alzheimer's disease is characterized by deposits of amyloid-beta (Abeta) peptides and neurofibrillary tangles. A decrease in the levels of Abeta peptide in neuronal and non-neuronal cells was observed with induced expression of PPARγ, or by activation of PPARγ using a thiazolidinedione (Camacho et al., J Neurosci. 2004, 24(48):10908-17). Treatment of APP7171 mice with PPARγ agonist pioglitazone showed several beneficial effects, including reduction in activated microglia and reactive astrocytes in the hippocampus and cortex, reduction in proinflammatory cyclooxygenase 2 and inducible nitric oxide synthase, decreased β-secretase-1 mRNA and protein levels, and a reduction in the levels of soluble Abeta1-42 peptide (Heneka et al., Brain. 2005, 128(Pt 6):1442-53).

Regions of degeneration of dopamine neurons in Parkinson's disease have been associated with increased levels of inflammatory cytokines (Nagatsu et al., J Neural Transm Suppl. 2000; (60):277-90). The effect of PPARγ agonist pioglitazone on dopaminergic nerve cell death and glial activation was studied in an MPTP mouse model of Parkinson's disease, wherein orally administered pioglitazone resulted in reduced glial activation as well as prevention of dopaminergic cell loss (Breidert et al. Journal of Neurochemistry, 2002, 82: 615).

(i) Other indications: PPARγ modulators have shown inhibition of VEGF-induced choroidal angiogenesis as well as repression of choroidal neovascularization effects, suggesting potential for treatment of retinal disorders. PPARδ has been shown to be expressed in implantation sites and in decidual cells in rats, suggesting a role in pregnancy, such as to enhance fertility. These studies were reviewed in Kota et al., 2005, Pharmacological Research 51: 85-94.

The management of pain, either neuropathic or inflammatory, is also suggested as a possible target for PPAR modulators. Burstein, S., Life Sci., 2005, 77(14):1674-84, suggests that PPARγ provides a receptor function for the activity of some cannabinoids. Lo Verme et al., Mol. Pharmacol. 2005, 67(1):15-9, identifies PPARα as a target responsible for pain and inflammation reducing effects of palmitoylethanolamide (PEA). PEA selectively activates PPARα in vitro, and induces expression of PPARα mRNA when applied topically to mice. In animal models of carrageenan-induced paw edema and phorbol ester-induced ear edema, inflammation in wild type mice is attenuated by PEA, which has no effect in PPARα deficient mice. PPARα agonists OEA, GW7647 and Wy-14643 demonstrate similar effects. Benani et al., Neurosci Lett. 2004, 369(1):59-63, uses a model of inflammation in rats to assess the PPAR response in the rat spinal cord following injection of complete Freund's adjuvant into the hind paw. It was shown that PPARα was activated, suggesting a role in pain pathways.

PPARs are also involved in some infections, and may be targeted in treating such infections. Dharancy et al. report that HCV infection is related to altered expression and function of the anti-inflammatory nuclear receptor PPARalpha, and identify hepatic PPARalpha as one mechanism underlying the pathogenesis of HCV infection, and as a new therapeutic target in traditional treatment of HCV-induced liver injury (Dharancy et al., Gastroenterology 2005, 128(2):334-42). J Raulin reports that among other effects, HIV infection induces alteration of cellular lipids, including deregulation of PPAR-gamma (J. Raulin, Prog Lipid Res 2002, 41(1):27-65). Slomiany and Slomiany report that PPARgamma activation leading to the impedance of Helicobacter pylori lipopolysaccharide (LPS) inhibitory effect on salivary mucin synthesis requires epidermal growth factor receptor (EGFR) participation. Further, they showed the impedance by ciglitazone was blunted in a concentration dependent fashion by a PPAR gamma agonist. (Slomiany & Slomiany, Inflammopharmacology 2004, 12(2): 177-88).

Muto et al. (Human Molecular Genetics 2002, 11(15):1731-1742) showed that molecular defects observed in Pkd1^{-/-} embryos contribute to the pathogenesis of autosomal dominant polycystic kidney disease (ADPKD) and that thiazolidindiones have a compensatory effect on the pathway affected by the loss of polycystin-1. Thus pathways activated by thiazolidinediones may provide new therapeutic targets in ADPKD (Muto et al., supra). Glintborg et al. show an increase in growth hormone levels in subjects with polycystic ovary syndrome treated with pioglitazone (Glintborg et al., J Clin Endocrinol Metab 2005, 90(10):5605-12).

In accordance with the description above, isoforms of the PPAR family of nuclear receptors are clearly involved in the systemic regulation of lipid metabolism and serve as “sensors” for fatty acids, prostanoid metabolites, eicosanoids and related molecules. These receptors function to regulate a broad array of genes in a coordinate fashion. Important biochemical pathways that regulate insulin action, lipid oxidation, lipid synthesis, adipocyte differentiation, peroxisome function, cell apoptosis, and inflammation can be modulated through the individual PPAR isoforms. Strong therapeutic effects of PPARα and PPARγ agonists to favorably influence systemic lipid levels, glucose homeostasis, and atherosclerosis risk (in the case of PPARα activation in humans) have recently been discovered. PPARα and PPARγ agonists are presently used clinically to favorably alter systemic lipid levels and glucose homeostasis, respectively. Recent observations made using PPARS ligands suggest that this isoform is also an important therapeutic target for dyslipidemia and insulin resistance, as well.

Thus, PPAR modulators, such as those described herein, can be used in the prophylaxis and/or therapeutic treatment of a variety of different disease and conditions, such as weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer.

### II. PPAR Active Compounds

As indicated in the Summary and in connection with applicable diseases and conditions, a number of different PPAR agonists have been identified. In addition, the present invention provides PPAR agonist compounds described by Formulae I, Ia, Ib, Ic or II as provided in the Summary above. These compounds can be used in the treatment or prophylaxis of a disease or condition selected from weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer.

The activity of the compounds can be assessed using methods known to those of skill in the art, as well as methods described herein. Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator. Similarly, when ligands to a target are sought, known ligands of the target can be present in control/calibration assay wells.

(a) Enzymatic Activity Assays

A number of different assays can be utilized to assess activity of PPAR modulators and/or determine specificity of a modulator for a particular PPAR. In addition to the assays mentioned in the Examples below, one of ordinary skill in the art will know of other assays that can be utilized and can modify an assay for a particular application. For example, the assay can utilize AlphaScreen (amplified luminescent proximity homogeneous assay) format, e.g., AlphaScreening system (Packard BioScience). AlphaScreen is generally described in Seethala and Prabhavathi, Homogenous Assays: AlphaScreen, Handbook of Drug Screening, Marcel Dekkar Pub. 2001, pp. 106-110. Applications of the technique to PPAR receptor ligand binding assays are described, for example, in Xu et al., Nature, 2002, 415:813-817.

(b) Assessment of Efficacy of Compounds in Disease Model Systems.

The utility of compounds of Formulae I, Ia, Ib, Ic, and II for the treatment of diseases such as autoimmune diseases and neurological diseases can be readily assessed using model systems known to those of skill in the art. For example, efficacy of PPAR modulators in models of Alzheimer's disease can be tested by mimicking inflammatory injury to neuronal tissues and measuring recovery using molecular and pharmacological markers (Heneka, et al., J. Neurosci., 2000, 20:6862-6867). Efficacy of PPAR modulators in multiple sclerosis has been monitored using the accepted model of experimental autoimmune encephalomyelitis (EAE) (Storer, et al., J. Neuroimmunol., 2004, 161:113-122. See also: Niino, et al., J, Neuroimmunol., 2001, 116:40-48; Diab, et al. J. Immunol., 2002, 168:2508-2515; Natarajan, et al., Genes Immun., 2002, 3:59-70; Feinstein, et al., Ann. Neurol., 2002, 51:694-702.)

(c) Isomers, Prodrugs, and Active Metabolites

Compounds contemplated herein are described with reference to both generic formulae and specific compounds. In addition, the invention compounds may exist in a number of different forms or derivatives, all within the scope of the present invention. These include, for example, tautomers, stereoisomers, racemic mixtures, regioisomers, salts, prodrugs (e.g., carboxylic acid esters), solvated forms, different crystal forms or polymorphs, and active metabolites.

(d) Tautomers, Stereoisomers, Regioisomers, and Solvated Forms

It is understood that some compounds may exhibit tautomerism. In such cases, the formulae provided herein expressly depict only one of the possible tautomeric forms. It is therefore to be understood that the formulae provided herein are intended to represent any tautomeric form of the depicted compounds and are not to be limited merely to the specific tautomeric form depicted by the drawings of the formulae.

Likewise, some of the compounds according to the present invention may exist as stereoisomers, i.e. having the same atomic connectivity of covalently bonded atoms yet differing in the spatial orientation of the atoms. For example, compounds may be optical stereoisomers, which contain one or more chiral centers, and therefore, may exist in two or more stereoisomeric forms (e.g. enantiomers or diastereomers). Thus, such compounds may be present as single stereoisomers (i.e., essentially free of other stereoisomers), racemates, and/or mixtures of enantiomers and/or diastereomers. As another example, stereoisomers include geometric isomers, such as cis- or trans-orientation of substituents on adjacent carbons of a double bond. All such single stereoisomers, racemates and mixtures thereof are intended to be within the scope of the present invention. Unless specified to the contrary, all such stereoisomeric forms are included within the formulae provided herein.

In some embodiments, a chiral compound of the present invention is in a form that contains at least 80% of a single isomer (60% enantiomeric excess (“e.e.”) or diastereomeric excess (“d.e.”)), or at least 85% (70% e.e. or d.e.), 90% (80% e.e. or d.e.), 95% (90% e.e. or d.e.), 97.5% (95% e.e. or d.e.), or 99% (98% e.e. or d.e.). As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. In some embodiments, the compound is present in optically pure form.

For compounds in which synthesis involves addition of a single group at a double bond, particularly a carbon-carbon double bond, the addition may occur at either of the double bond-linked atoms. For such compounds, the present invention includes both such regioisomers.

Additionally, the formulae are intended to cover solvated as well as unsolvated forms of the identified structures. For example, the indicated structures include both hydrated and non-hydrated forms. Other examples of solvates include the structures in combination with a suitable solvent, such as isopropanol, ethanol, methanol, DMSO, ethyl acetate, acetic acid, or ethanolamine.

(e) Prodrugs and Metabolites

In addition to the present formulae and compounds described herein, the invention also includes prodrugs (generally pharmaceutically acceptable prodrugs), active metabolic derivatives (active metabolites), and their pharmaceutically acceptable salts.

Prodrugs are compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. In this context, a common example is an alkyl ester of a carboxylic acid.

As described in The Practice of Medicinal Chemistry, Ch. 31-32 (Ed. Wermuth, Academic Press, San Diego, Calif., 2001), prodrugs can be conceptually divided into two non-exclusive categories, bioprecursor prodrugs and carrier prodrugs. Generally, bioprecursor prodrugs are compounds that are inactive or have low activity compared to the corresponding active drug compound, that contain one or more protective groups and are converted to an active form by metabolism or solvolysis. Both the active drug form and any released metabolic products should have acceptably low toxicity. Typically, the formation of active drug compound involves a metabolic process or reaction that is one of the follow types:

Oxidative reactions: Oxidative reactions are exemplified without limitation to reactions such as oxidation of alcohol, carbonyl, and acid functionalities, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions.

Reductive reactions: Reductive reactions are exemplified without limitation to reactions such as reduction of carbonyl functionalitites, reduction of alcohol functionalities and carbon-carbon double bonds, reduction of nitrogen-containing functional groups, and other reduction reactions.

Reactions without change in the oxidation state: Reactions without change in the state of oxidation are exemplified without limitation to reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. (See, e.g., Cheng et al., U.S. Patent Publ. No. 20040077595, application Ser. No. 10/656,838, incorporated herein by reference.) Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols. Wermuth, supra.

Prodrugs may proceed from prodrug form to active form in a single step or may have one or more intermediate forms which may themselves have activity or may be inactive.

Metabolites, e.g., active metabolites, overlap with prodrugs as described above, e.g., bioprecursor prodrugs. Thus, such metabolites are pharmacologically active compounds or compounds that further metabolize to pharmacologically active compounds that are derivatives resulting from metabolic processes in the body of a subject. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug. Metabolites of a compound may be identified using routine techniques known in the art, and their activities determined using tests such as those described herein. For example, in some compounds, one or more alkoxy groups can be metabolized to hydroxyl groups while retaining pharmacologic activity and/or carboxyl groups can be esterified, e.g., glucuronidation. In some cases, there can be more than one metabolite, where an intermediate metabolite(s) is further metabolized to provide an active metabolite. For example, in some cases a derivative compound resulting from metabolic glucuronidation may be inactive or of low activity, and can be further metabolized to provide an active metabolite.

Prodrugs and active metabolites may be identified using routine techniques known in the art. See, e.g., Bertolini et al., 1997, J. Med. Chem., 40:2011-2016; Shan et al., 1997, J Pharm Sci 86(7):756-757; Bagshawe, 1995, Drug Dev. Res., 34:220-230; Wermuth, supra.

(f) Pharmaceutically Acceptable Salts

Compounds can be formulated as or be in the form of pharmaceutically acceptable salts. Contemplated pharmaceutically acceptable salt forms include, without limitation, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug. A compound of the invention may possess a sufficiently acidic, a sufficiently basic, or both functional groups, and accordingly react with any of a number of inorganic or organic bases, and inorganic and organic acids, to form a pharmaceutically acceptable salt.

Pharmaceutically acceptable salts include acid addition salts such as those containing sulfate, pyrosulfate, bisulfate, sulfite, bisulfite, chloride, bromide, iodide, hydrochloride, fumarate, maleate, phosphate, monohydrogenphosphate, dihydrogenphosphate, metaphosphate, pyrophosphate, sulfamate, acetate, citrate, lactate, tartrate, sulfonate, methanesulfonate, propanesulfonate, ethanesulfonate, benzenesulfonate, p-toluenesulfonate, naphthalene-1-sulfonate, naphthalene-2-sulfonate, xylenesulfonates, cyclohexylsulfamate, quinate, propionate, decanoate, caprylate, acrylate, formate, isobutyrate, caproate, heptanoate, propiolate, oxalate, malonate, succinate, suberate, sebacate, fumarate, maleate, butyne-1,4 dioate, hexyne-1,6-dioate, benzoate, chlorobenzoate, methylbenzoate, dinitrobenzoate, hydroxybenzoate, methoxybenzoate, phthalate, phenylacetate, phenylpropionate, phenylbutyrate, gamma-hydroxybutyrate, glycollate, and mandelate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, Pa., Vol. 2, p. 1457, 1995. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. In another example, a salt can be prepared by reacting the free base and acid in an organic solvent.

Thus, for example, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

Unless specified to the contrary, specification of a compound herein includes pharmaceutically acceptable salts of such compound.

(g) Polymorphic Forms

In the case of agents that are solids, it is understood by those skilled in the art that the compounds and salts may exist in different crystal or polymorphic forms, all of which are intended to be within the scope of the present invention and specified formulae.

### II. Administration

The methods and compounds will typically be used in therapy for human subjects. However, they may also be used to treat similar or identical indications in other animal subjects. In this context, the terms “subject”, “animal subject”, and the like refer to human and non-human vertebrates, e.g., mammals such as non-human primates, sports and commercial animals, e.g., bovines, equines, porcines, ovines, rodents, and pets e.g., canines and felines.

Suitable dosage forms, in part, depend upon the use or the route of administration, for example, oral, transdermal, transmucosal, inhalant, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in Remington: The Science and Practice of Pharmacy, 21^{st} edition, Lippincott, Williams and Wilkins, Philadelphia, Pa., 2005 (hereby incorporated by reference herein).

Compounds of the present invention (i.e. Formula I, including Formulae Ia-Ic, and Formula II, and all sub-embodiments disclosed herein) can be formulated as pharmaceutically acceptable salts.

Carriers or excipients can be used to produce compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, transdermal, or inhalant. In some embodiments, oral administration is preferred. For oral administration, for example, the compounds can be formulated into conventional oral dosage forms such as capsules, tablets, and liquid preparations such as syrups, elixirs, and concentrated drops.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, poly-vinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compounds of the invention are formulated in sterile liquid solutions, preferably in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this invention are formulated preferably as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Creams for topical application are preferably formulated from a mixture of mineral oil, self-emulsifying beeswax and water in which mixture the active ingredient, dissolved in a small amount solvent (e.g., an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

For inhalants, compounds of the invention may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The compounds of the invention may also be used in combination with other inhaled therapies, for example corticosteroids such as fluticasone proprionate, beclomethasone dipropionate, triamcinolone acetonide, budesonide, and mometasone furoate; beta agonists such as albuterol, salmeterol, and formoterol; anticholinergic agents such as ipratroprium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

The amounts of various compounds to be administered can be determined by standard procedures taking into account factors such as the compound EC₅₀, the biological half-life of the compound, the age, size, and weight of the subject, and the disorder associated with the subject. The importance of these and other factors are well known to those of ordinary skill in the art. Generally, a dose will be between about 0.01 and 50 mg/kg, preferably 0.1 and 20 mg/kg of the subject being treated. Multiple doses may be used.

The compounds of the invention may also be used in combination with other therapies for treating the same disease. Such combination use includes administration of the compounds and one or more other therapeutics at different times, or co-administration of the compound and one or more other therapies. In some embodiments, dosage may be modified for one or more of the compounds of the invention or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than a compound of the present invention, or at the same time as a compound of the invention. Use in combination also includes use with a therapy or medical procedure that is administered once or infrequently, such as surgery, along with a compound of the invention administered within a short time or longer time before or after the other therapy or procedure. In some embodiments, the present invention provides for delivery of compounds of the invention and one or more other drug therapeutics delivered by a different route of administration or by the same route of administration. The use in combination for any route of administration includes delivery of compounds of the invention and one or more other drug therapeutics delivered by the same route of administration together in any formulation, including formulations where the two compounds are chemically linked in such a way that they maintain their therapeutic activity when administered. In one aspect, the other drug therapy may be co-administered with one or more compounds of the invention. Use in combination by co-administration includes administration of co-formulations or formulations of chemically joined compounds, or administration of two or more compounds in separate formulations within a short time of each other (e.g. within an hour, 2 hours, 3 hours, up to 24 hours), administered by the same or different routes. Co-administration of separate formulations includes co-administration by delivery via one device, for example the same inhalant device, the same syringe, etc., or administration from separate devices within a short time of each other. Co-formulations of compounds of the invention and one or more additional drug therapies delivered by the same route includes preparation of the materials together such that they can be administered by one device, including the separate compounds combined in one formulation, or compounds that are modified such that they are chemically joined, yet still maintain their biological activity. Such chemically joined compounds may have a linkage that is substantially maintained in vivo, or the linkage may break down in vivo, separating the two active components.

### IV. Synthesis of Compounds of Formula I

The generic methodology to prepare compounds as described by Formula Ic where Q is O, R³ and R⁴ are H and R¹ is COOH is illustrated in Scheme 1. Analogs can be prepared in a 3-step process.

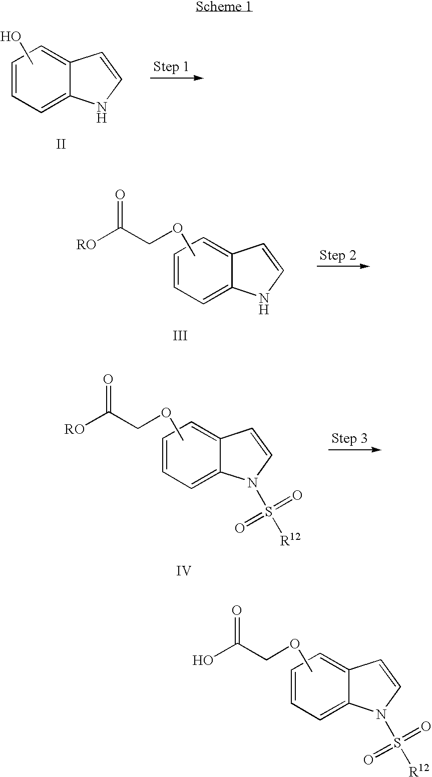

Step 1: Preparation of Compound III

From 4- or 5-hydroxy indole II, compound III can be prepared through coupling of the hydroxyl group with bromoacetic acid ester (methyl or ethyl) under basic conditions in a solvent system, such as for example, acetonitrile.

Step 2: Preparation of Compound IV

Compound IV can be prepared through coupling of a sulfonyl chloride with the indole III under basic conditions in a solvent system, such as for example, N,N-Dimethylformamide (DMF).

Step 3: Saponification of the Ester

The preparation of the compounds as described by Formula I where Q is O; R³ and R⁴ are H, and R¹ is COOH can be achieved through deprotection of ester (methyl or ethyl) IV under saponification conditions with an aqueous hydroxide solution in an inert solvent, such as for example, tetrahydrofuran (THF).

The generic methodology to prepare compounds as described by Formula Ic where Q is CH₂, R³ and R⁴ are H, and R¹ is COOH is illustrated in Scheme 2. Analogs can be prepared in a 4-step process

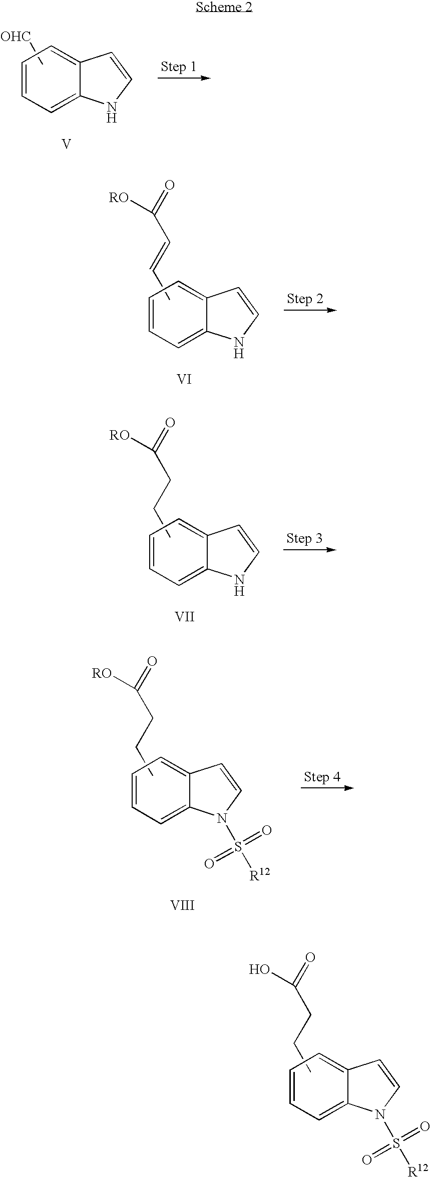

Step 1: Preparation of Compound VI

Compound VI can be prepared via the coupling of the aldehyde moiety V with an Horner-Wadsworth Emmons reagent under basic conditions in an inert solvent, such as for example, THF.

Step 2: Preparation of Compound VII

Compound VII can be prepared through reduction of the acrylic acid ester VI under catalytic hydrogenation conditions with a hydrogenation catalyst, such as for example, palladium on activated carbon, in an inert solvent, such as for example, THF.

Step 3: Preparation of Compound VIII

Compound VIII can be prepared through coupling of a sulfonyl chloride with the indole VII under basic conditions in a solvent such as DMF.

Step 4: Saponifcation of the Ester

The preparation of the compounds as described by Formula Ic where Q is CH₂, R³ and R⁴ are H, and R¹ is COOH can be achieved through deprotection of the ester (methyl or ethyl) VIII under saponification conditions with an aqueous hydroxide solution with an inert solvent, such as for example THF.

An alternative approach to generate compounds where Q is CH₂ is described in Scheme 3, which also provides a 4 step process. In the alternative route, the sulfonylation step is the first step, and synthesis of the propionic acid is achieved in the next three steps.

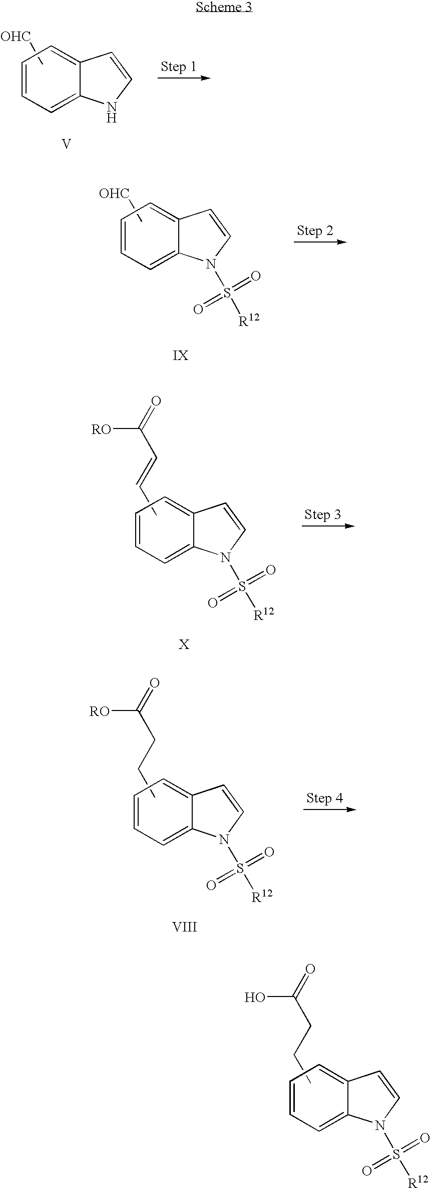

Step 1: Preparation of Compound IX

Compound IX can be prepared through coupling of a sulfonyl chloride with the indole V under aqueous basic conditions in a solvent such as dichloromethane, utilizing a phase transfer catalyst such as tetrabutylammonium hydrogen sulfate, or via coupling with the aid of a base, such as for example, sodium hydride, in an inert solvent such as DMF.

Step 2: Preparation of Compound X

Compound X can be prepared via coupling of the aldehyde moiety IX with a Horner-Wadsworth Emmons reagent under basic conditions in an inert solvent such as THF.

Step 3: Preparation of Compound VIII

Compound VIII can be prepared via reduction of the acrylic acid ester X under catalytic hydrogenation conditions, employing a hydrogenation catalyst, such as for example, palladium on activated carbon, in an inert solvent such as THF.

Step 4: Saponification of the Ester

The preparation of the compounds as described by Formula Ic where Q is CH₂, R³ and R⁴ are H, and R¹ is COOH can be achieved through deprotection of ester (methyl or ethyl) VIII under saponification conditions with an aqueous hydroxide solution with an inert solvent such as THF.

Another variation to the preparation of compounds as described in Formula Ic where Q is CH₂ is illustrated in Scheme 4 with a 3 step process.

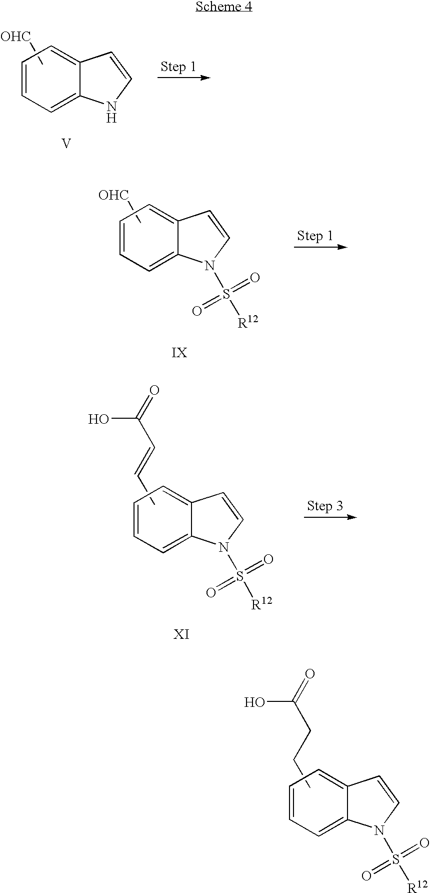

Step 1: Preparation of Compound IX

Compound IX can be prepared via coupling of a sulfonyl chloride with the indole V under basic conditions in a solvent such as dichloromethane, utilizing a phase transfer catalyst such as tetrabutylammonium hydrogen sulfate.

Step 2: Preparation of Compound XI

Compound XI can be prepared through coupling of aldehyde IX with malonic acid to produce the acrylic acid.

Step 3: Preparation of Propionic Acid.

The preparation of the compounds as described by Formula Ic where Q is CH₂, R³ and R⁴ are H, and R¹ is COOH can be achieved through reduction of the acrylic acid XI under catalytic hydrogenation conditions with a hydrogenation catalyst, such as for example, palladium on activated carbon in an inert solvent such as THF.

### EXAMPLES

### Example 1

### Synthesis of 3-{1-[5-(3-Trifluoromethoxy-phenyl)-thiophene-2-sulfonyl]-1H-indol-5-yl}-propionic acid (6)

Compound 6 was synthesized in 5 steps from 5-formylindole (1) as shown in Scheme 5.

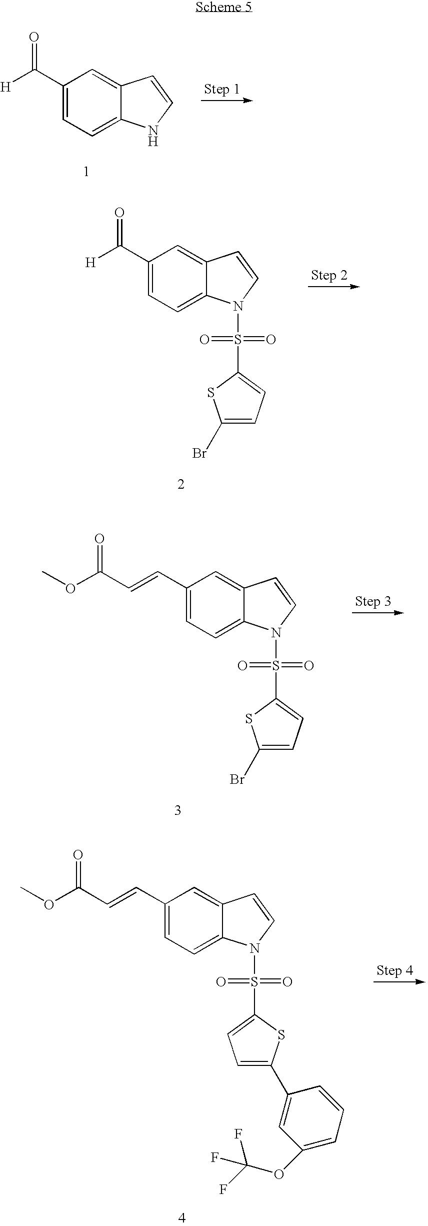

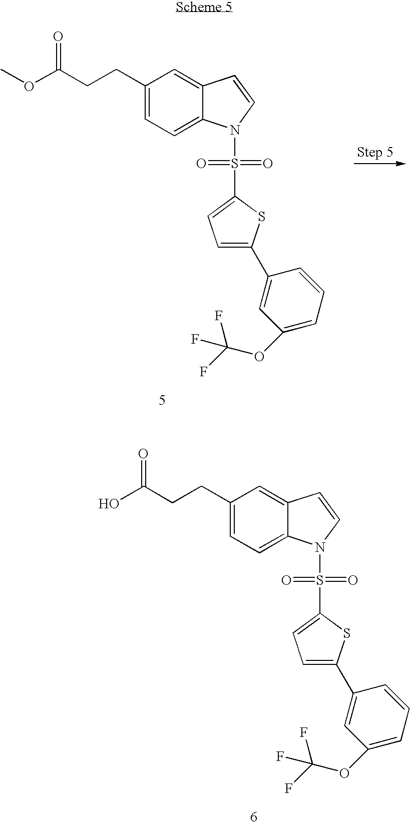

### Step-1—Preparation of 1-(5-bromo-thiophene-sulfonyl)-1H-indol-5-carbaldehyde (2)

To a stirred solution of 5-formylindole (1, 1.6 g, 10.1 mmol) in methylene chloride (280 mL) and 50% potassium hydroxide in water (78 mL) was added 5-bromo-thiophenesulfonyl chloride (2.8 g, 12.0 mmol) and a catalytic amount of tetra-n-butyl ammonium hydrogen sulfate. The reaction mixture was stirred overnight at 25° C. The organic layer was washed with saturated sodium bicarbonate, dried over magnesium sulfate and filtered. Removal of the solvent under vacuum yielded a light yellow solid (2, 2.3 g, 57% yield). MS (ESI) [M+H⁺]⁺=370.1; 372.1.

### Step-2—Preparation of (E)-3-[1-(5-Bromo-thiophene-2-sulfonyl)-1H-indol-5-yl]-acrylic acid methyl ester (3)

Methyl diethylphosphonoacetate (1.71 mL, 9.3 mmol) in THF (6 mL) was cooled to 0° C. and sodium hydride (253 mg, 10.6 mmol) was added. The mixture was stirred at 0° C. for 15 min and the solution of deprotonated methyl diethylphosphonoacetate was added dropwise to a stirred solution of 1-(5-bromo-thiophene-sulfonyl)-H-indol-5-carbaldehyde (2, 2.3 g, 6.21 mmol) in THF (22 mL) at 0° C. After slowly warming to room temperature and stirring for 72 h, ethyl acetate was added to the reaction mixture. The organic layer was separated, washed with saturated sodium bicarbonate, dried over magnesium sulfate and filtered. Concentration under reduced pressure resulted in a brown oil which was subsequently filtered over a bed of silica (25% ethyl acetate in hexanes). Removal of solvent resulted in an off-white solid (3, 2.4 g, 91% yield). MS (ESI) [M+H⁺]⁺=427.1

### Step-3—Preparation of (E)-3-{1-(5-(3-Trifluoromethoxy-phenyl)-thiophene-2-sulfonyl]-1H-indol-5-yl}-acrylic acid methyl ester (4)

To a stirred solution of (E)-3-[1-(5-bromo-thiophene-2-sulfonyl)-1H-indol-5-yl]-acrylic acid methyl ester (3, 50.0 mg, 0.12 mmol) in THF (3.0 mL) was added 3-trifluoromethoxyphenylboronic acid (53.1 mg, 0.26 mmol), 1N K₂CO₃ (0.2 mL) and tetrakis(triphenylphosphine)palladium(0) (4.0 mg, 0.034 mmol). The reaction was heated at 110° C. in the microwave for 45 min. Ethyl acetate was added to the reaction mixture and was washed with saturated sodium bicarbonate, dried over magnesium sulfate and filtered. Concentration under reduced pressure afforded the crude material, which was purified by column chromatography (30% ethyl acetate in hexanes) to yield the desired product as a white solid (4, 37 mg, 62% yield). MS (ESI) [M+H⁺]⁺=508.1

### Step-4—Preparation of 3-{1-[5-(3-Trifluoromethoxy-phenyl)-thiophene-2-sulfonyl]-1H-indol-5-yl}-propionic acid methyl ester (5)

To a stirred solution of (E)-3-{1-(5-(3-trifluoromethoxy-phenyl)-thiophene-2-sulfonyl]-1H-indol-5-yl}-acrylic acid methyl ester (4, 37 mg, 0.07 mmol) in methanol (0.5 mL) was added 10% palladium on activated carbon. After the reaction flask was degassed, the mixture was stirred overnight under a hydrogen atmosphere at 25° C. The solution was filtered over a bed of Celite and the filtrate was concentrated under reduced pressure to yield a brown solid (5, 35 mg, 97% yield). MS (ESI) [M+H⁺]⁺=510.2

### Step-5—Preparation of 3-{1-[5-(3-Trifluoromethoxy-phenyl)-thiophene-2-sulfonyl]-1H-indol-5-yl}-propionic acid (6)

To a solution of 3-{1-[5-(3-trifluoromethoxy-phenyl)-thiophene-2-sulfonyl]-1H-indol-5-yl}-propionic acid methyl ester (5, 190 mg, 0.041 mmol) in THF (2.0 mL), was added 1 M lithium hydroxide (0.75 mL). After stirring for overnight at 25° C., ethyl acetate was added and the mixture was acidified with 1M hydrochloric acid. The organic layer was separated, dried over magnesium sulfate, filtered and concentrated at reduced pressure to obtain a light brown solid (6 24 mg, 82% yield). MS (ESI) [M−H⁺]⁻=494.2.

### Example 2

### Synthesis of {1-[5-(1-Methyl-5-trifluoromethyl-1H-pyrazol-3-yl)-thiophene-2-sulfonyl]-1H-indol-5-yl oxy}acetic acid (7)

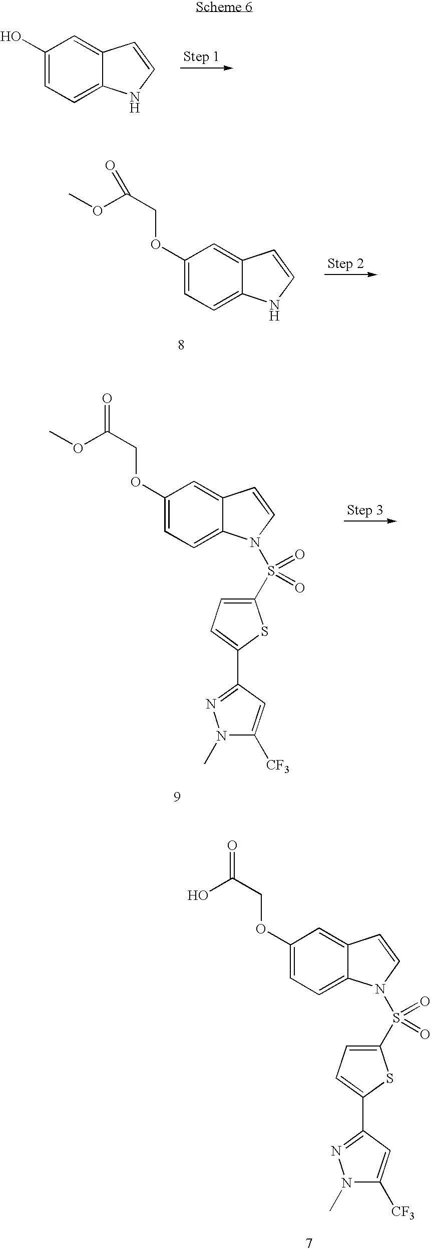

Compound 7 was synthesized in 2 steps from (1H-indol-5-yloxy)-acetic acid methyl ester 8 as shown on Scheme 6.

### Step 1: Preparation of (1H-indol-5-yloxy)-acetic acid ethyl ester (8)

5-Hydroxyindole (498 mg, 3.7 mmol), potassium carbonate (1.5 g, 11.0 mmol) and ethyl bromoacetate (1.0 g, 6.0 mmol) were dissolved in 2-butanone (55 mL) under an inert atmosphere. A condenser was attached and the reaction heated at 89° C. overnight. The reaction was cooled and filtered. Solvent was removed by evaporation to give desired product as crude (8, 800 mg, 97%).

### Step 2—Preparation of {1-[5-(1-Methyl-5-trifluoromethyl-1H-pyrazol-3-yl)-thiophene-2-sulfonyl]-1H-indol-5-yl oxy}acetic acid methyl ester (9)

(1H-indol-5-yloxy)-acetic acid ethyl ester (8, 85.0 mg, 0.41 mmol) was dissolved in DMF (8 mL) under an argon atmosphere. Sodium hydride (18.2 mg, 0.45 mmol) was added, and when the evolution of gas had ceased 5-(1-Methyl-5-trifluoromethyl-1H-pyrazol-3-yl)-thiophene-2-sulfonyl chloride (102 mg, 0.31 mmol) added. The reaction was stirred overnight at room temperature under an argon blanket. The organic layer was extracted with ethyl acetate (2×30 mL) in water (30 mL), washed with brine and dried over anhydrous magnesium sulfate. Desired product was isolated after flash chromatography (silica gel; gradient solvent system 0-10% ethyl acetate/hexane).

### Step 3—Synthesis of {1-[5-(1-Methyl-5-trifluoromethyl-1H-pyrazol-3-yl)-thiophene-2-sulfonyl]-1H-indol-5-yl oxy}acetic acid (7)

The methyl ester (9) was dissolved in a 4:1 ratio of THF:aqueous potassium hydroxide (1M) [1 mg/ml], and was stirred at ambient temperature for 5 hours. The reaction mixture was diluted with water, acidified to pH 1-2 with 1N HCl, and then extracted with ethyl acetate. The combined organic layers was then washed with brine, and dried over sodium sulfate. Evaporation of the solvent yielded an off-white solid (7, 7 mg, 4.7%). MS (ESI) [M+H⁺]⁺=486.20 [M−H⁺]⁻=484.10

### Example 3

### Synthesis of {1-[4-(4-Trifluoromethylphenoxy)-benzenesulfonyl]-1H-indol-5-yl oxy}acetic acid (10) and [1-(4-Methoxybenzenesulfonyl)-1H-indol-5 yloxy]acetic acid (11)

Using the synthetic schemes described above, a set of exemplary compounds 10 ((MS(ESI) [M−H⁺]⁻=490.11) and 11 (MS (ESI) [M−H⁺]⁻=360.05) were prepared. See Table 1 for compound structures.

Exemplary compounds described by Formula I are provided in Table I and in the synthetic examples. Additional compounds of Formula I, Ia, Ib, Ic or II can be prepared and tested to confirm activity using conventional methods and the guidance provided herein.

TABLE 1
Exemplary compounds of the invention.

| Compound | | | Expected | Experimental |
|---|---|---|---|---|
| Number | Structure | Name | MW | MW |
| 12 | 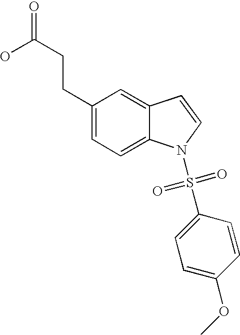 | 3-[1-(4-Methoxy-benzenesulfonyl)-1H-indol-5-yl]-propionic acid | 359.40 | MS (ESI)[M − H⁺]⁻ =368.1 |
| 13 | 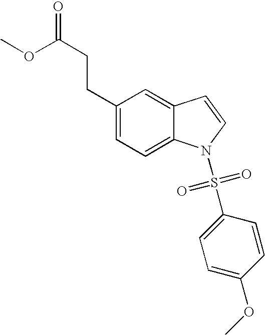 | 3-[1-(4-Methoxy-benzenesulfonyl)-1H-indol-5-yl]-propionic acidMethyl ester | 373.43 | MS (ESI)[M + H⁺]⁺ =374.2 |
| 14 | 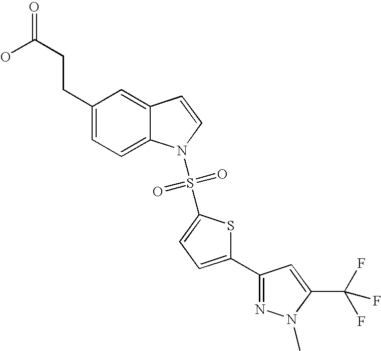 | 3-{1-[5-(1-Methyl-5-trifluoromethyl-1H-pyrazol-3-yl)-thiophene-2-sulfonyl]-1H-indol-5-yl}-propionic acid | 483.49 | MS (ESI)[M − H⁺]⁻ =482.1 |
| 10 | 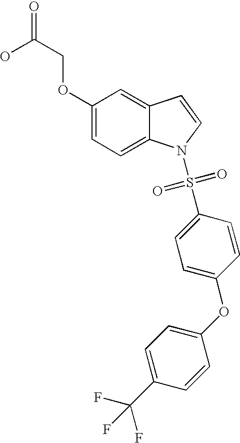 | {1-[4-(4-Trifluoromethyl-phenoxy)-benzenesulfonyl]-1H-indol-5-yloxy}-acetic acid | 491.44 | MS (ESI)[M − H⁺]⁻ =490.11 |
| 7 | 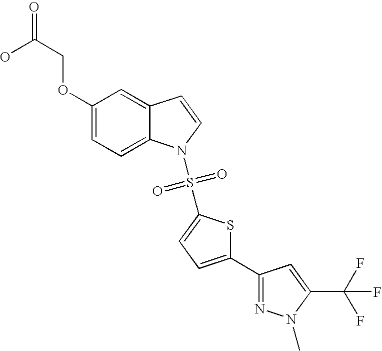 | {1-[5-(1-Methyl-5-trifluoromethyl-1-H-pyrazol-3-yl)-thiophene-2-sulfonyl]-1H-indol-5-yloxy}-acetic acid | 485.46 | MS (ESI)[M − H⁺]⁻ =484.10 |
| 11 | 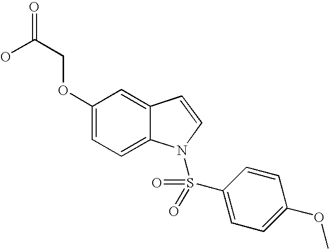 | [1-(4-Methoxy-benzenesulfonyl)-1H-indol-5-yloxy]-acetic acid | 361.37 | MS (ESI)[M − H⁺]⁻ =360.05 |
| 15 | 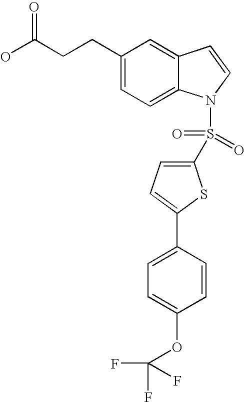 | 3-{1-[5-(4-Trifluoro-methoxy-phenyl)-thiophene-2-sulfonyl]-1H-indol-5-yl}-propionic acid | 495.50 | MS(M − H+) =494.2 |
| 6 | 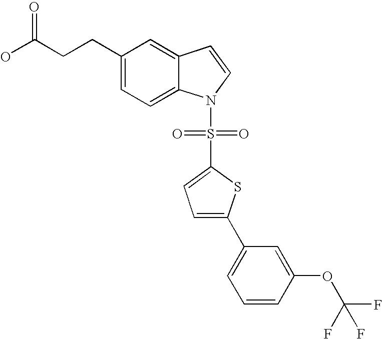 | 3-{1-[5-(3-Trifluoro-methoxy-phenyl)-thiophene-2-sulfonyl]-1H-indol-5-yl}-propionic acid | 495.50 | MS (ESI)[M − H⁺]⁻ =494.2. |

### Example 4

### Expression and Purification of PPARs for Use in Biochemical and Cell Assays

Genetic Engineering

Plasmids encoding the Ligand-binding domains (LBDs) of PPARα, PPARγ, and PPARδ were engineered using common polymerase chain reaction (PCR) methods (pGal4-PPARα-LBD, pGal4-PPARγ-LBD, pGal4-PPARδ-LBD). The relevant DNA sequences and encoded protein sequences used in the assay are shown for each (see below). Complementary DNA cloned from various human tissues were purchased from Invitrogen, and these were used as substrates in the PCR reactions. Specific custom synthetic oligonucleotide primers (Invitrogen, see below) were designed to initiate the PCR product, and also to provide the appropriate restriction enzyme cleavage sites for ligation with the plasmids.

The plasmids used for ligation with the receptor-encoding inserts were either pET28 (Novagen) or a derivative of pET28, pET-BAM6, for expression using E. coli. In each of these cases the receptor LBD was engineered to include a Histidine tag for purification using metal affinity chromatography.

Protein Expression and Purification of PPAR's.

For protein expression, plasmids containing genes of interest were transformed into E. coli strain BL21 (DE3)RIL (Invitrogen) and transformants selected for growth on LB agar plates containing appropriate antibiotics. Single colonies were grown for 4 hrs at 37° C. in 200 ml LB media. For PPARα and PPARγ all protein expression was performed by large scale fermentation using a 30 L bioreactor. 400 ml of starter culture was added to 30 L TB culture and allowed to grow at 37° C. until an OD600 nm of 2-5 was obtained. The culture was cooled to 20° C. and 0.5 mM IPTG added, the culture was allowed to grow for a further 18 hrs.

For PPARδ protein expression, single colonies were grown for 4 hrs at 37° C. in 200 ml LB media. 16×1 L of fresh TB media in 2.8 L flasks were inoculated with 10 ml of starter culture and grown with constant shaking at 37° C. Once cultures reached an absorbance of 1.0 at 600 nm, an additive to improve the solubility of the PPARδ was added to the culture and 30 min later, 0.5 mM IPTG was added and cultures allowed to grow for a further 12 to 18 hrs at 20° C. Cells were harvested by centrifugation and pellets frozen at −80° C. until ready for lysis/purification.

For protein purification; all operations were carried out at 4° C. Frozen E. coli cell pellets were resuspended in lysis buffer and lysed using standard mechanical methods. Soluble proteins were purified via poly-Histidine tags using immobilized metal affinity purification (IMAC). For each of the PPAR's described all have been purified using a 3 step purification process utilizing IMAC, size exclusion chromatography and ion exchange chromatography. For PPARα the poly-Histidine tag was optionally removed using Thrombin (Calbiochem). In the case of PPARδ, during protein purification the solubility improving additive was present in order to maintain protein stability. During the final step of purification solubility improving additives were desalted away before concentration.

Plasmid Sequence and PCR Primer Information:

| PPARα (Nucleic acid SEQ ID NO:_{——}) (Protein SEQ ID NO:_{——}) | | | |
|---|---|---|---|
| P332. pET28 PPARA E199-Y468-X | | | |
| taatacgactcactataggggaattgt | | | |
| gagcggataacaattcccctctagaaataattttgtttaactttaagaaggagatatacc | | | |
| atgggcagcagccatcatcatcatcatcacagcagcggcctggtgccgcgcggcagccat | | | |
| M G S S H H H H H H S S G L V P R G S H | | | |
| atggaaactgcagatctcaaatctctggccaagagaatctacgaggcctacttgaagaac | | | |
| M E T A D L K S L A K R I Y E A Y L K N | | | |
| ttcaacatgaacaaggtcaaagcccgggtcatcctctcaggaaaggccagtaacaatcca | | | |
| F N M N K V K A R V I L S G K A S N N P | | | |
| ccttttgtcatacatgatatggagacactgtgtatggctgagaagacgctggtggccaag | | | |
| P F V I H D M E T L C M A E K T L V A K | | | |
| ctggtggccaatggcatccagaacaaggaggcggaggtccgcatctttcactgctgccag | | | |
| L V A N G I Q N K E A E V R I F H C C Q | | | |
| tgcacgtcagtggagaccgtcacggagctcacggaattcgccaaggccatcccaggcttc | | | |
| C T S V E T V T E L T E F A K A I P G F | | | |
| gcaaacttggacctgaacgatcaagtgacattgctaaaatacggagtttatgaggccata | | | |
| A N L D L N D Q V T L L K Y G V Y E A I | | | |
| ttcgccatgctgtcttctgtgatgaacaaagacgggatgctggtagcgtatggaaatggg | | | |
| F A M L S S V M N K D G M L V A Y G N G | | | |
| tttataactcgtgaattcctaaaaagcctaaggaaaccgttctgtgatatcatggaaccc | | | |
| F I T R E F L K S L R K P F C D I M E P | | | |
| aagtttgattttgccatgaagttcaatgcactggaactggatgacagtgatatctccctt | | | |
| K F D F A M K F N A L E L D D S D I S L | | | |
| tttgtggctgctatcatttgctgtggagatcgtcctggccttctaaacgtaggacacatt | | | |
| F V A A I I C C G D R P G L L N V G H I | | | |
| gaaaaaatgcaggagggtattgtacatgtgctcagactccacctgcagagcaaccacccg | | | |
| E K M Q E G I V H V L R L H L Q S N H P | | | |
| gacgatatctttctcttcccaaaacttcttcaaaaaatggcagacctccggcagctggtg | | | |
| D D I F L F P K L L Q K M A D L R Q L V | | | |
| acggagcatgcgcagctggtgcagatcatcaagaagacggagtcggatgctgcgctgcac | | | |
| T E H A Q L V Q I I K K T E S D A A L H | | | |
| ccgctactgcaggagatctacagggacatgtactgagtcgacaagcttgcggccgcactc | | | |
| P L L Q E I Y R D M Y - | | | |
| gagcaccaccaccaccaccactgagat | | | |
| PCR primers: | | | |
| PPARA | PPARA-S | GCTGACACATATGGAAACTGCAGATCTCAAATC | (SEQ ID NO:_{——}) |
| | PPARA-A | GTGACTGTCGACTCAGTACATGTCCCTGTAGA | (SEQ ID NO:_{——}) |
| PPARγ: (Nucleic acid SEQ ID NO:_{——}) (Protein SEQ ID NO:_{——}) | | | |
| P333. pET28 PPARG E205-Y475-X | | | |
| taatacgactcactataggggaattgt | | | |
| gagcggataacaattcccctctagaaataattttgtttaactttaagaaggagatatacc | | | |
| atgggcagcagccatcatcatcatcatcacagcagcggcctggtgccgcgcggcagccat | | | |
| M G S S H H H H H H S S G L V P R G S H | | | |
| atggagtccgctgacctccgggccctggcaaaacatttgtatgactcatacataaagtcc | | | |
| M E S A D L R A L A K H L Y D S Y I K S | | | |
| ttcccgctgaccaaagcaaaggcgagggcgatcttgacaggaaagacaacagacaaatca | | | |
| F P L T K A K A R A I L T G K T T D K S | | | |
| ccattcgttatctatgacatgaattccttaatgatgggagaagataaaatcaagttcaaa | | | |
| P F V I Y D M N S L M M G E D K I K F K | | | |
| cacatcacccccctgcaggagcagagcaaagaggtggccatccgcatctttcagggctgc | | | |
| H I T P L Q E Q S K E V A I R I F Q G C | | | |
| cagtttcgctccgtggaggctgtgcaggagatcacagagtatgccaaaagcattcctggt | | | |
| Q F R S V E A V Q E I T E Y A K S I P G | | | |
| tttgtaaatcttgacttgaacgaccaagtaactctcctcaaatatggagtccacgagatc | | | |
| F V N L D L N D Q V T L L K Y G V H E I | | | |
| atttacacaatgctggcctccttgatgaataaagatggggttctcatatccgagggccaa | | | |
| I Y T M L A S L M N K D G V L I S E G Q | | | |
| ggcttcatgacaagggagtttctaaagagcctgcgaaagccttttggtgactttatggag | | | |
| G F M T R E F L K S L R K P F G D F M E | | | |
| cccaagtttgagtttgctgtgaagttcaatgcactggaattagatgacagcgacttggca | | | |
| P K F E F A V K F N A L E L D D S D L A | | | |
| atatttattgctgtcattattctcagtggagaccgcccaggtttgctgaatgtgaagccc | | | |
| I F I A V I I L S G D R P G L L N V K P | | | |
| attgaagacattcaagacaacctgctacaagccctggagctccagctgaagctgaaccac | | | |
| I E D I Q D N L L Q A L E L Q L K L N H | | | |
| cctgagtcctcacagctgtttgccaagctgctccagaaaatgacagacctcagacagatt | | | |
| P E S S Q L F A K L L Q K M T D L R Q I | | | |
| gtcacggaacatgtgcagctactgcaggtgatcaagaagacggagacagacatgagtctt | | | |
| V T E H V Q L L Q V I K K T E T D M S L | | | |
| cacccgctcctgcaggagatctacaaggacttgtactaggtcgacaagcttgcggccgca | | | |
| H P L L Q E I Y K D L Y - | | | |
| ctcgagcaccaccaccaccaccactgagat | | | |
| PCR Primers: | | | |
| PPARG | PPARG-S | GCTCAGACATATGGAGTCCGCTGACCTCCGGGC | (SEQ ID NO:_{——}) |
| | PPARG-A | GTGACTGTCGACCTAGTACAAGTCCTTGTAGA | (SEQ ID NO:_{——}) |
| PPARδ: (Nucleic acid SEQ ID NO:_{——}) (Protein SEQ ID NO:_{——}) | | | |
| P1057. pET BAM6 PPARD G165-Y441-X | | | |
| taatacgactcactataggggaattgt | | | |
| gagcggataacaattcccctctagaaataattttgtttaactttaagaaggagatatacc | | | |
| atgaaaaaaggtcaccaccatcaccatcacggatcccagtacaacccacaggtggccgac | | | |
| M K K G H H H H H H G S Q Y N P Q V A D | | | |
| ctgaaggccttctccaagcacatctacaatgcctacctgaaaaacttcaacatgaccaaa | | | |
| L K A F S K H I Y N A Y L K N F N M T K | | | |
| aagaaggcccgcagcatcctcaccggcaaagccagccacacggcgccctttgtgatccac | | | |
| K K A R S I L T G K A S H T A P F V I H | | | |
| gacatcgagacattgtggcaggcagagaaggggctggtgtggaagcagttggtgaatggc | | | |
| D I E T L W Q A E K G L V W K Q L V N G | | | |
| ctgcctccctacaaggagatcagcgtgcacgtcttctaccgctgccagtgcaccacagtg | | | |
| L P P Y K E I S V H V F Y R C Q C T T V | | | |
| gagaccgtgcgggagctcactgagttcgccaagagcatccccagcttcagcagcctcttc | | | |
| E T V R E L T E F A K S I P S F S S L F | | | |
| ctcaacgaccaggttacccttctcaagtatggcgtgcacgaggccatcttcgccatgctg | | | |
| L N D Q V T L L K Y G V H E A I F A M L | | | |
| gcctctatcgtcaacaaggacgggctgctggtagccaacggcagtggctttgtcacccgt | | | |
| A S I V N K D G L L V A N G S G F V T R | | | |
| gagttcctgcgcagcctccgcaaacccttcagtgatatcattgagcctaagtttgaattt | | | |
| E F L R S L R K P F S D I I E P K F E F | | | |
| gctgtcaagttcaacgccctggaacttgatgacagtgacctggccctattcattgcggcc | | | |
| A V K F N A L E L D D S D L A L F I A A | | | |
| atcattctgtgtggagaccggccaggcctcatgaacgttccacgggtggaggctatccag | | | |
| I I L C G D R P G L M N V P R V E A I Q | | | |
| gacaccatcctgcgtgccctcgaattccacctgcaggccaaccaccctgatgcccagtac | | | |
| D T I L R A L E F H L Q A N H P D A Q Y | | | |
| ctcttccccaagctgctgcagaagatggctgacctgcggcaactggtcaccgagcacgcc | | | |
| L F P K L L Q K M A D L R Q L V T E H A | | | |
| cagatgatgcagcggatcaagaagaccgaaaccgagacctcgctgcaccctctgctccag | | | |
| Q M M Q R I K K T E T E T S L H P L L Q | | | |
| gagatctacaaggacatgtactaagtcgaccaccaccaccaccaccactgagatccggct | | | |
| E I Y K D M Y - | | | |
| ggccctactggccgaaaggaattcgaggccagcagggccaccgctgagcaataactagca | | | |
| taaccccttggggcctctaaacgggtcttgaggggttttttg | | | |
| PCR Primers: | | | |
| PPARD | PPARD-G165 | GTTGGATCCCAGTACAACCCACAGGTGGC | (SEQ ID NO:_{——}) |
| | PPARD-A | GTGACTGTCGACTTAGTACATGTCCTTGTAGA | (SEQ ID NO:_{——}) |

### Example 5

### Bio-Chemical Screening

The homogenous Alpha screen assay was used in the agonist mode to determine the ligand dependent interaction of the PPARs (α,δ,γ) with the coactivator Biotin-PGC-1 peptide (biotin-AHX-DGTPPPQEAEEPSLLKKLLLAPANT-CONH₂ (SEQ ID NO:______), supplied by Wyeth). All compounds tested were serially diluted 1:3 into DMSO for a total of 8 concentration points. Samples were prepared with His-tagged PPAR-LBD prepared per Example 21. Ni-chelate acceptor beads were added that bind to the his-tagged PPAR-LBD and streptavidin donor beads were added that bind to the biotin of the coactivator (Perkin-Elmer #6760619M) such that agonist activity correlates to signal from the donor and acceptor beads in close proximity. Each sample was prepared by mixing 1 μl of compound and 15 μl of 1.33× receptor/peptide mix, incubating for 15 minutes at room temperature, then adding 4 μl of 4× beads in assay buffer. The assay buffer was 50 mM HEPES, pH 7.5, 50 mM KCl, 1 mM DTT and 0.8% BSA. Final concentrations for each sample were 25 nM biotin-PGC-1 peptide, 20 nM PPARγ or 10 nM PPARα or δ, and each bead at 5 μg/ml, with compound added to the desired concentration resulting in final DMSO of 5%. WY-14643(PPARα), farglitazar (PPARγ) and bezafibrate (PPARδ) were assayed as control samples. The samples were incubated for 1 hour in the dark at room temperature before taking the reading in the Fusion alpha or Alpha Quest reader. The signal vs. compound concentration was used to determine the EC₅₀. The data was expressed in μMol/L. The data points from the Fusion alpha instrument were transferred to Assay Explorer® (MDL) to generate a curve and calculate the inflection point of the curve as EC₅₀.

### Example 6

### Co-Transfection Assay

This assay serves to confirm the observed biochemical activity (Example 22) on the modulation of intended target molecule(s) at the cellular level. 293T cells (ATCC) were seeded at 1-2×10⁶ cells per well of a 6 well plate (Corning 3516) in 3 ml of growth medium (Dulbecco's eagle medium, Mediatech, with 10% FBS). These were incubated to 80-90% confluent and the medium was removed by aspirating. These cells were transfected with PPAR LBD and luciferase such that agonist results in activation of the luciferase. Measurement of luciferase activity of transfected cells treated with compounds directly correlates with agonist activity. To 100 μl of serum free growth medium was added 1 μg of pFR-Luc (Stratagene catalog number 219050), 6 μl Metafectene (Biontex, Inc.) and 1 mg of the pGal4-PPAR-LBD (α, γ or δ from Example 21). This was mixed by inverting, then incubated for 15-20 minutes at room temperature, and diluted with 900 μl of serum free growth medium. This was overlayed onto the 293T cells and incubated for 4-5 hours at 37° C. in CO₂ incubator. The transfection medium was removed by aspirating and growth medium was added and the cells incubated for 24 hours. The cells were then suspended in 5 ml of growth medium and diluted with an additional 15 ml of growth medium. For each test sample, 95 μl of the transfected cells were transferred per well of a 96 well culture plate. Compounds tested were diluted in DMSO to 200× the desired final concentration. This was diluted 10× with growth medium and 5 μl was added to the 95 μl of transfected cells. The plate was incubated for 24 hours 37° C. in CO₂ incubator. Luciferase reaction mixture was prepared by mixing 1 ml of lysis buffer, 1 ml of substrate in lysis buffer, and 3 ml of reaction buffer (Roche Diagnostics Luciferase assay kit #1814036). For each sample well, the growth medium was replaced with 50 ml of reaction mixture and the plate shaken for 15-20 minutes, and the luminescence was measured on a Victor2 V plate reader (Perkin Elmer). The signal vs. compound concentration was used to determine the EC₅₀.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to exemplary compounds of Formula I, Ia, Ib, Ic or II to provide additional active compounds. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

### PPAR Sequences

PPARA Accession No. NM_005036 (SEQ ID NO:_{——})
gcgccgcctc cttcggcgtt cgccccacgg accggcaggc ggcggaccgc ggcccaggct
gaagctcagg gccctgtctg ctctgtggac tcaacagttt gtggcaagac aagctcagaa
ctgagaagct gtcaccacag ttctggaggc tgggaagttc aagatcaaag tgccagcaga
ttcagtgtca tgtgaggacg tgcttcctgc ttcatagata agagtagctt ggagctcggc
ggcacaacca gcaccatctg gtcgcgatgg tggacacgga aagcccactc tgccccctct
ccccactcga ggccggcgat ctagagagcc cgttatctga agagttcctg caagaaatgg
gaaacatcca agagatttcg caatccatcg gcgaggatag ttctggaagc tttggcttta
cggaatacca gtatttagga agctgtcctg gctcagatgg ctcggtcatc acggacacgc
tttcaccagc ttcgagcccc tcctcggtga cttatcctgt ggtccccggc agcgtggacg
agtctcccag tggagcattg aacatcgaat gtagaatctg cggggacaag gcctcaggct
atcattacgg agtccacgcg tgtgaaggct gcaagggctt ctttcggcga acgattcgac
tcaagctggt gtatgacaag tgcgaccgca gctgcaagat ccagaaaaag aacagaaaca
aatgccagta ttgtcgattt cacaagtgcc tttctgtcgg gatgtcacac aacgcgattc
gttttggacg aatgccaaga tctgagaaag caaaactgaa agcagaaatt cttacctgtg
aacatgacat agaagattct gaaactgcag atctcaaatc tctggccaag agaatctacg
aggcctactt gaagaacttc aacatgaaca aggtcaaagc ccgggtcatc ctctcaggaa
aggccagtaa caatccacct tttgtcatac atgatatgga gacactgtgt atggctgaga
agacgctggt ggccaagctg gtggccaatg gcatccagaa caaggaggcg gaggtccgca
tctttcactg ctgccagtgc acgtcagtgg agaccgtcac ggagctcacg gaattcgcca
aggccatccc aggcttcgca aacttggacc tgaacgatca agtgacattg ctaaaatacg
gagtttatga ggccatattc gccatgctgt cttctgtgat gaacaaagac gggatgctgg
tagcgtatgg aaatgggttt ataactcgtg aattcctaaa aagcctaagg aaaccgttct
gtgatatcat ggaacccaag tttgattttg ccatgaagtt caatgcactg gaactggatg
acagtgatat ctcccttttt gtggctgcta tcatttgctg tggagatcgt cctggccttc
taaacgtagg acacattgaa aaaatgcagg agggtattgt acatgtgctc agactccacc
tgcagagcaa ccacccggac gatatctttc tcttcccaaa acttcttcaa aaaatggcag
acctccggca gctggtgacg gagcatgcgc agctggtgca gatcatcaag aagacggagt
cggatgctgc gctgcacccg ctactgcagg agatctacag ggacatgtac tgagttcctt
cagatcagcc acaccttttc caggagttct gaagctgaca gcactacaaa ggagacgggg
gagcagcacg attttgcaca aatatccacc actttaacct tagagcttgg acagtctgag
ctgtaggtaa ccggcatatt attccatatc tttgttttaa ccagtacttc taagagcata
gaactcaaat gctgggggta ggtggctaat ctcaggactg ggaagattac ggcgaattat
gctcaatggt ctgattttaa ctcacccgat gttaatcaat gcacattgct ttagatcaca
ttcgtgattt accatttaat taactggtaa cctcaaaatt cgtggcctgt cttcccattc
accccgcttt tgactattgt gctcctttat aattctgaaa actaatcagc actttttaac
aatgtttata atcctataag tctagatgta tccaaaggtg aagtatgtaa aaagcagcaa
aatatttatt tcaaagactt cacttctgtt tcctgaatct aaagaaagac aacatgctgc
tttttaatca taggatggag aattttaaag aactgtttgg gccaggcaca gtcgctcata
cttgtaatcc cagcactttg ggaggccgag gcgggtggat cacaaggtca gcagatcgag
accatcctgg ccaacatggt gaaaccctgt ctctactaaa aatacaaaaa ttagccgggt
gtggtggcac atgcctgtaa tcccagctac tcgggaagct gaggcaggag aattgcttga
accagggagt tggaggttgc agtgagctaa gactgcacca ctgcactcca gcctggtgac
agaacgagac tctgtcttaa aaacaaacaa acaaaaaaaa aatctgttag ataagctatc
aaaatgcagc tgttgttttg tttttggctc actgttttcg tggttgtaac taatatgtgg
aaaggcccat ttccaggttt gcgtagaaga gcccagaaaa cagagtctca agacccccgc
tctggactgt cataagctag cacccgtggt aagcgggacg agacaagctc ccgaagcccg
ccagcttcct gctccactca gctccgtcca gtcaacctga acccacccag tccagctgtc
tgtgggaatg gtggtgttct tagggacaga ctgacacctt acttgtcagt gttcctccgg
gccccatttg gcagctcccg tatcttttgt tatgttgctt ttaaagatat gatgttttat
tgttttaact cttggtgaca gtagatgctc tctggagcgc agacgaggca catgtgtctt
catagcctgg gctgggtggg agccagtcac cctgcggatc gagagagggg gtagagtctt
cttcaaatgg cagttttact tcaaatggca gatttcacaa gagttggtta ttttttacaa
tggtttaggt tgttaagtct cctttgtatg taaggtagtt ttttcaacat ctaaaatttt
tgttttagcc ttcaaaacca acttaccaac ctcagtccag ctgggaaggc agcgttgatt
atggtagttt gtcaagaata tatggacctg gaaacacttt ctctctctgt ccacctggta
gataaattgt cctgttgaga atttttagat ctggactgga actgccagga ccaccgcctc
cagggagtcg ctgggcacct ggaggtatcg tcgatgcctc tcccccatct ttagaaaatt
tggctcttct gaggtcatta ttattttaag aatgattagg attgataagg gtcccatgac
cagcattatg aaaatgcgag agtgggaagg acacagtgtg agacttccac tagaaaaaag
tgaaagttag ggttaggaca tcctttttta aaaattacaa atttagtccg ttttggtttt
tgtaatcagg ctaggcacag tggctcacac atggaatccc agcactttgg gaggccgagg
tgggaggatc acttgagccc aggagttcga gaccagccta ggcaacatag caagaccctg
tctgtacaca aaatttaaaa attagttcat cggggtggca cacatcagta gtcccagcta
ctctgcaggc tgaggtggga ggattgcttg aacccaggag gtcgaggctg cagtgagctg
tgatctcacc actgcattcc agcctgggtg acagagttag attccaccct ctcccacccc
ggcaaaaaaa aaaaaaaaag atgcaatcaa aggggctgtt ggccagcaat ggcagcagca
gcggcgggca gtctgcccaa gtgtcttagg aaccaaaagc aaataaaagt gtttccatat
atgccaccag ccaagtggcc atcctaattc agaaagaagc tagcctttga gtgtctgtca
tggtgcatcc gtttcagtat tatttcctaa aatgagaagc ccctgtgtca acaagatcca
ggggctggag cccaatgcca agcctgtgtt gtccccagcg accctgcagc tgctcgctct
gatgtaccct gtgccattca aggagatgtg gtccaggaaa gtgagcctca tggttttcag
agaagtcatt gttctgttta cattttcata aaacctgttt aaaatagctc cccgtctcag
gctttcagca gtaacagtga gctgactggc aagttcgatg ttagctcccg ggacactcag
cagcgatggt gagcattttg gtttccttaa ggcccagcaa gacttccagg gacatctctg
gtgaagccag aatggagaca cccgtgacct caggctgaaa gtcactcgac attggtctct
tgtgttgata gggaaggaaa tcaggcattc ctatttcttt aaataacaaa accactaatt
gccactcaat gctggaatat tttgggtcac ctaatcatag atttctcagg gcatcaatac
tcaaatatag gctgattatg ccccagttca aatgggaact attaacagag tgcatttctt
gcttgctggg tttcaacaga catcagccaa aagaacaaaa gagatgtcag gacagattcc
aggagtgtcg gagcacatgt gtggcacccg ctccctctgg cagcgaatgt aggaagtcgc
caaatttacc cactcttcaa caagtcattg tttaaacacg gtttttcatt ttctcaactt
ttaatagcaa aaagtgccaa agtcctcaga gacctaacag ccttggtcta ccgtgctgac
cagggtgaag gcacggcgag ggactcctcc cagacgtgcc tcttgtgtgc cagctggctg
tggctcggga gcagacgcag gcctctccat tgtccagggg agcctggcgg cgcatccctc
ctctcccacc tcctggcact tccagctggg tgtcccacat gttggattcc gtccccacca
cacttccaga gaccggagaa ctgtgcaggg cctaaggccg tttggatgaa ttgtcaaaac
aagatgcttc cagttacagc ggcaggagcg ggactgggag cacgggctga cggctgctgg
tgcctttctt cccacctcgc ttgcctgttt ccgcttgacc cttcctccag ctccgatgag
aagagtataa agcatcttcc taacgggtgt gtttgctata cgaacataat ggacgtgaag
tggggcagaa acccagaact cagcattcaa ggatgcccag gagagctgtc cctgttttaa
agagctgtgt tttgttttgt ttcgcattta gagagcagac aaggcaccct tctgctgcgc
tgatacgttt cttacactgg gccattttag acccccaggg aaacagcctt cctggagcgt
tgtctggagg ttccagggac agggcagcct cccagagccg agcaagagct caaggtacaa
atgagagatt tgctataccg tgagaagtca acaacttagc caccacttcc ccgcaatgga
ccatgtaaca aatacctcag caggccctgc aaaaggccat gctagagctg aggcgcacag
cctgtggcct ctgtagttag ggcaggtggg atggagactc cttgagtgca cacacctgag
cctgcccaca cacaggggag cagcatctcg tatgacgtct ggaaggaact tcggttgtgt
aaagggagcc ttgaagatac gtgcaaaagg tgctacccca atttggtgaa actgacattg
ggcacgtctt gggcttagga gaagcggccg atggtcccgg cctgcagtga caaacccccc
tccccgcacc gcccccagca ccccctctcc tcttcacctc ttcctgctgg ccacgaggaa
gccacttcct cagagagacc ctaccagatg cggatggaaa cagatgcacc aaagcaagcc
ctgatgaaac cgcgacttcc taaggtctgt ctcctctgaa cttgcacctg ggcctctctg
tgtttggttc caagcacttc ccacctcaaa ctcccatttt caaaccactg tatctctgcg
cacatctgct acttaccagc cgcatacatg atggagggtt ttttggtcct gatccagtgg
ccacacctgt ctttgaaatg tctcactgaa ctccagtttt aaaatagatt cattgcttca
acacagcaag cccaatgcac ccagctaaga ctggcttgac cgacagcctg gcctttggtg
gggggcttcc tggggcctgg ggaaagctgg ccaccttcaa cagctggtac ctcttcaaca
gtgtggcctt tcaaaatgca gatgccacca ggagaacatg cccacagctc accacctatg
gatgccatgg ctctgggcag ctttcaaagc aggttcctgt ggtctcctca gctgtttgag
ggggtaacag caaatcagcc tccattttaa aatgaaaaca ccagcctcca gatgtagggc
ctgctgggtg ttgctagccg ctggtcccca ggcacggtgc actttctcca cctcctgcag
cctccctgtt gtttctagac tcttgcacct ggtgagtgca aggataggtg acccaggggc
ctgcagcctt gtcctcagct cccatctcct ggactgccag cctcaccctc tgcagttagc
atggttggcc tgatgcaggg atcccgaggg attacttttt agaccttctt tcacattcag
aaaagtagta tagattcagg agaggcaaga aaattatgct gtccatagaa gtcacccatg
aagactgatg ccaccacctg aaggctcatg attgttaaaa atgtccacgg gaacctctcg
tccacaggag gtttgtctca acacttccca tttttacggc attggcattg ccaagcatgg
ggaagtatct gctcttctca tgttaaaagt ggcccagctt ttcttaactc agtccaagct
gacttgttta gctgcactgg aatttcttac caaccaaata tttgcatcga gcaaaggggg
ctgtgtgcac ctccctaatg gcagcgatga tggctgctgt cattcaagcc catcttcaga
cgtcacagtc tggaagtgaa atgtccacaa acatctgtgg cagaaaaggc tatacggacc
acccagttgt gctgcagctt tacagagcaa ggaagggttg tggcaaataa atgattaacc
tgcctcgact gtgctgaggg caacaaaggc catctcacca aaggattatt cgatgccatt
aaatcatccc gtgaccttcc tgcttccgag tccatggcct ttgcccaggg catgtactcc
cctgagaggc cttctgccta gaaagatcta tgactgggtt ccaaagttga ggcctaggtt
tttgctggga tttagatatt ttcaggcacc attttgacag cattcaggaa aacggttatt
gaccccatag actagggtaa gaataaaggc aataaatttg gtctgactca gaatatagga
gatccatata tttctctgga aaccacagtg tacactaaaa tgtgaaattg aaggttttgt
taaaaagaaa aagataatga gcttcatgct ttgtttaatt acataatgat ttccattacg
ctatttctgt gaaatgcagc aggttcttaa acgttatttc agtggcatgg gctggaagct
tatcacaaaa agccatgtgt gtggccttat cagaacagaa agagacaggc tggtgcccaa
ggctgctgcc tgctccacct tttgccagct ctggacatct gaggacgtcc cggcagatct
ggaatggggc cctcaactga ccatttgctt ctcagaattt cagtttgaga catgagaggt
ataatcagtt acttttctcc ccccagagaa acccttttgt gaggggagag gagctatggt
atgtggttca gctgaaacac atacaactgc atccttttgg agtcctttgc caacaaaaac
agaccaacag accagatggt gtccatgttc aatatcatgt cttgatggac gcagctgatg
acctcaaata cttgagtggt ctcatggctg ttagatggat tatttgaaaa aaaaaaaaaa
aaaagagaga aaaaataatt gatttttaca tcagagatag caaactaaga cctggggagg
ggggtcagct tttattttat tttatttttt ttaagtttgc tagttgggtc aaatgtgagg
aggagggagt ctacctgcca cctcttctct tgcccctctt ctgcccacac atccagcatc
caaaatccat tcatttaatg aattgataaa gtgccgtgca aactggtgca caaacaggcc
cccagtccac gcagcctggc tcctaggaaa agtggtgacc gggcgtgggg gggcatgccg
cagccctggg acacagtcgg gcaccttccc cggaccccca ggccttggct gtgcctcaag
tcagagaggg tcagccttca ggccccggag acgagtgact ggccgatcat ttcacaataa
aatcactcac ttttggcaac ttcacttttt ttaaggcaca gtcagttcct tttctcatgt
acctcacaaa agatgaagac catgtagtac tctttttggt aaagttacag tgttcatgtt
aaatatcact tttttctaca ttgtgtggta aaaagaacta cgttaatagc tatatcttaa
atactgtgat ttgacttttt gaaaaatatc ctaatacaaa tattttacta acttacaatc
actcatttaa taagaaacat ttggattctt ttgaaatcag tgttaattga ctcatattct
taaaagcctg gctcttgacc ctattggaaa cacaaaggaa gctgaaatca aacatctaaa
atacactgcg tacacgtgtg cgtgcacaca cacacacaca cacacacaca cacagctctt
catttctcct gagccatgca gaatttactt tcaatgtgga aatctgttcc ctttaccaca
ctgtatatgc acagagcaca agagaggcta tctctagtca cttccaccag cgaggcctta
gactccgtat tagaggccac cgatttcata caacagtgtt tcgctaaaga cccttcacta
ttcttgttta gtaaatagct gtctgctctt cagggaactg ttacctatgg gttattacca
aagaacgctg gcaattggaa atgtcctgat ggaaattctt tgcacgtgcc ggttctctgg
catcctccag gtggcccaac ccaaagcaga aagcagaaac cacagacccc gtgagtctcc
ccataccttg tttccaataa cttggcaaaa cttcttggtg catattggtt acaccctctg
ggattcataa tgccattagg ctaaaaccct aagagagagg gttgacagaa acacacgcga
gaatgaggca gatcccagag caaggactgg gcccagactc tccacatgtg ctctactagt
gagtgcctta tactctcagt attttggggc ttacagcttc ttatttgtgc taaaaaggtg
cagttccaaa gtaggaactg ccacacaggc cccagcatcc tctctccaac ttcatacctc
tctcctggtg gggggagcgg gcatccagga cctccggaat caaggatgtg cagagaagag
cgaaagtaat ttttctagtc acatgaactg attggttcca ggcaattaga aaatggctat
aaaataacct taattttaaa aaaaaatctt gggtcttcgt tttcctatta ggagactgaa
ctgaccacat gtattgattt atatcctgaa tatatgggaa cttctgtgtt tgggatgtcc
tactgtaaga ctgatgaatg tacagagtta atttcagggt acagttttgc cttaatggtt
ttaaaaaata aactattttt taaaatttt
PPARA Accession No. NP_005027 (SEQ ID NO:_{——})
MVDTESPLCP LSPLEAGDLE SPLSEEFLQE MGNIQEISQS IGEDSSGSFG FTEYQYLGSC
PGSDGSVITD TLSPASSPSS VTYPVVPGSV DESPSGALNI ECRICGDKAS GYHYGVHACE
GCKGFFRRTI RLKLVYDKCD RSCKIQKKNR NKCQYCRFHK CLSVGMSHNA IRFGRMPRSE
KAKLKAEILT CEHDIEDSET ADLKSLAKRI YEAYLKNFNM NKVKARVILS GKASNNPPFV
IHDMETLCMA EKTLVAKLVA NGIQNKEAEV RIFHCCQCTS VETVTELTEF AKAIPGFANL
DLNDQVTLLK YGVYEAIFAM LSSVMNKDGM LVAYGNGFIT REFLKSLRKP FCDIMEPKFD
FAMKFNALEL DDSDISLFVA AIICCGDRPG LLNVGHIEKM QEGIVHVLRL HLQSNHPDDI
flfpkllqkm adlrqlvteh aqlvqiikkt esdaalhpll qeiyrdmy
PPARG Accession No. NM_015869 (SEQ ID NO:_{——})
actgatgtct tgactcatgg gtgtattcac aaattctgtt acttcaagtc tttttctttt
aacggattga tcttttgcta gatagagaca aaatatcagt gtgaattaca gcaaacccct
attccatgct gttatgggtg aaactctggg agattctcct attgacccag aaagcgattc
cttcactgat acactgtctg caaacatatc acaagaaatg accatggttg acacagagat
gccattctgg cccaccaact ttgggatcag ctccgtggat ctctccgtaa tggaagacca
ctcccactcc tttgatatca agcccttcac tactgttgac ttctccagca tttctactcc
acattacgaa gacattccat tcacaagaac agatccagtg gttgcagatt acaagtatga
cctgaaactt caagagtacc aaagtgcaat caaagtggag cctgcatctc caccttatta
ttctgagaag actcagctct acaataagcc tcatgaagag ccttccaact ccctcatggc
aattgaatgt cgtgtctgtg gagataaagc ttctggattt cactatggag ttcatgcttg
tgaaggatgc aagggtttct tccggagaac aatcagattg aagcttatct atgacagatg
tgatcttaac tgtcggatcc acaaaaaaag tagaaataaa tgtcagtact gtcggtttca
gaaatgcctt gcagtgggga tgtctcataa tgccatcagg tttgggcgga tgccacaggc
cgagaaggag aagctgttgg cggagatctc cagtgatatc gaccagctga atccagagtc
cgctgacctc cgggccctgg caaaacattt gtatgactca tacataaagt ccttcccgct
gaccaaagca aaggcgaggg cgatcttgac aggaaagaca acagacaaat caccattcgt
tatctatgac atgaattcct taatgatggg agaagataaa atcaagttca aacacatcac
ccccctgcag gagcagagca aagaggtggc catccgcatc tttcagggct gccagtttcg
ctccgtggag gctgtgcagg agatcacaga gtatgccaaa agcattcctg gttttgtaaa
tcttgacttg aacgaccaag taactctcct caaatatgga gtccacgaga tcatttacac
aatgctggcc tccttgatga ataaagatgg ggttctcata tccgagggcc aaggcttcat
gacaagggag tttctaaaga gcctgcgaaa gccttttggt gactttatgg agcccaagtt
tgagtttgct gtgaagttca atgcactgga attagatgac agcgacttgg caatatttat
tgctgtcatt attctcagtg gagaccgccc aggtttgctg aatgtgaagc ccattgaaga
cattcaagac aacctgctac aagccctgga gctccagctg aagctgaacc accctgagtc
ctcacagctg tttgccaagc tgctccagaa aatgacagac ctcagacaga ttgtcacgga
acacgtgcag ctactgcagg tgatcaagaa gacggagaca gacatgagtc ttcacccgct
cctgcaggag atctacaagg acttgtacta gcagagagtc ctgagccact gccaacattt
cccttcttcc agttgcacta ttctgaggga aaatctgaca cctaagaaat ttactgtgaa
aaagcatttt aaaaagaaaa ggttttagaa tatgatctat tttatgcata ttgtttataa
agacacattt acaatttact tttaatatta aaaattacca tattatgaaa aaaaaaaaaa
aaa
PPARG Accession No. NP_056953 (SEQ ID NO:_{——})
MGETLGDSPI DPESDSFTDT LSANISQEMT MVDTEMPFWP TNFGISSVDL SVMEDHSHSF
DIKPFTTVDF SSISTPHYED IPFTRTDPVV ADYKYDLKLQ EYQSAIKVEP ASPPYYSEKT
QLYNKPHEEP SNSLMAIECR VCGDKASGFH YGVHACEGCK GFFRRTIRLK LIYDRCDLNC
RIHKKSRNKC QYCRFQKCLA VGMSHNAIRF GRMPQAEKEK LLAEISSDID QLNPESADLR
ALAKHLYDSY IKSFPLTKAK ARAILTGKTT DKSPFVIYDM NSLMMGEDKI KFKHITPLQE
QSKEVAIRIF QGCQFRSVEA VQEITEYAKS IPGFVNLDLN DQVTLLKYGV HEIIYTMLAS
LMNKDGVLIS EGQGFMTREF LKSLRKPFGD FMEPKFEFAV KFNALELDDS DLAIFIAVII
LSGDRPGLLN VKPIEDIQDN LLQALELQLK LNHPESSQLF AKLLQKMTDL RQIVTEHVQL
LQVIKKTETD MSLHPLLQEI YKDLY
PPARD Accession No. NM_006238 (SEQ ID NO:_{——})
gcggagcgtg tgacgctgcg gccgccgcgg acctggggat taatgggaaa agttttggca
ggagcgggag aattctgcgg agcctgcggg acggcggcgg tggcgccgta ggcagccggg
acagtgttgt acagtgtttt gggcatgcac gtgatactca cacagtggct tctgctcacc
aacagatgaa gacagatgca ccaacgaggc tgatgggaac caccctgtag aggtccatct
gcgttcagac ccagacgatg ccagagctat gactgggcct gcaggtgtgg cgccgagggg
agatcagcca tggagcagcc acaggaggaa gcccctgagg tccgggaaga ggaggagaaa
gaggaagtgg cagaggcaga aggagcccca gagctcaatg ggggaccaca gcatgcactt
ccttccagca gctacacaga cctctcccgg agctcctcgc caccctcact gctggaccaa
ctgcagatgg gctgtgacgg ggcctcatgc ggcagcctca acatggagtg ccgggtgtgc
ggggacaagg catcgggctt ccactacggt gttcatgcat gtgaggggtg caagggcttc
ttccgtcgta cgatccgcat gaagctggag tacgagaagt gtgagcgcag ctgcaagatt
cagaagaaga accgcaacaa gtgccagtac tgccgcttcc agaagtgcct ggcactgggc
atgtcacaca acgctatccg ttttggtcgg atgccggagg ctgagaagag gaagctggtg
gcagggctga ctgcaaacga ggggagccag tacaacccac aggtggccga cctgaaggcc
ttctccaagc acatctacaa tgcctacctg aaaaacttca acatgaccaa aaagaaggcc
cgcagcatcc tcaccggcaa agccagccac acggcgccct ttgtgatcca cgacatcgag
acattgtggc aggcagagaa ggggctggtg tggaagcagt tggtgaatgg cctgcctccc
tacaaggaga tcagcgtgca cgtcttctac cgctgccagt gcaccacagt ggagaccgtg
cgggagctca ctgagttcgc caagagcatc cccagcttca gcagcctctt cctcaacgac
caggttaccc ttctcaagta tggcgtgcac gaggccatct tcgccatgct ggcctctatc
gtcaacaagg acgggctgct ggtagccaac ggcagtggct ttgtcacccg tgagttcctg
cgcagcctcc gcaaaccctt cagtgatatc attgagccta agtttgaatt tgctgtcaag
ttcaacgccc tggaacttga tgacagtgac ctggccctat tcattgcggc catcattctg
tgtggagacc ggccaggcct catgaacgtt ccacgggtgg aggctatcca ggacaccatc
ctgcgtgccc tcgaattcca cctgcaggcc aaccaccctg atgcccagta cctcttcccc
aagctgctgc agaagatggc tgacctgcgg caactggtca ccgagcacgc ccagatgatg
cagcggatca agaagaccga aaccgagacc tcgctgcacc ctctgctcca ggagatctac
aaggacatgt actaacggcg gcacccaggc ctccctgcag actccaatgg ggccagcact
ggaggggccc acccacatga cttttccatt gaccagccct tgagcacccg gcctggagca
gcagagtccc acgatcgccc tcagacacat gacacccacg gcctctggct ccctgtgccc
tctctcccgc ttcctccagc cagctctctt cctgtctttg ttgtctccct ctttctcagt
tcctctttct tttctaattc ctgttgctct gtttcttcct ttctgtaggt ttctctcttc
ccttctccct tgccctccct ttctctctcc accccccacg tctgtcctcc tttcttattc
tgtgagatgt tttgtattat ttcaccagca gcatagaaca ggacctctgc ttttgcacac
cttttcccca ggagcagaag agagtggggc ctgccctctg ccccatcatt gcacctgcag
gcttaggtcc tcacttctgt ctcctgtctt cagagcaaaa gacttgagcc atccaaagaa
acactaagct ctctgggcct gggttccagg gaaggctaag catggcctgg actgactgca
gccccctata gtcatggggt ccctgctgca aaggacagtg ggcaggaggc cccaggctga
gagccagatg cctccccaag actgtcattg cccctccgat gctgaggcca cccactgacc
caactgatcc tgctccagca gcacacctca gccccactga cacccagtgt ccttccatct
tcacactggt ttgccaggcc aatgttgctg atggccccct gcactggccg ctggacggca
ctctcccagc ttggaagtag gcagggttcc ctccaggtgg gcccccacct cactgaagag
gagcaagtct caagagaagg aggggggatt ggtggttgga ggaagcagca cacccaattc
tgcccctagg actcggggtc tgagtcctgg ggtcaggcca gggagagctc ggggcaggcc
ttccgccagc actcccactg cccccctgcc cagtagcagc cgcccacatt gtgtcagcat
ccagggccag ggcctggcct cacatccccc tgctcctttc tctagctggc tccacgggag
ttcaggcccc actccccctg aagctgcccc tccagcacac acacataagc actgaaatca
ctttacctgc aggctccatg cacctccctt ccctccctga ggcaggtgag aacccagaga
gaggggcctg caggtgagca ggcagggctg ggccaggtct ccggggaggc aggggtcctg
caggtcctgg tgggtcagcc cagcacctgc tcccagtggg agcttcccgg gataaactga
gcctgttcat tctgatgtcc atttgtccca atagctctac tgccctcccc ttccccttta
ctcagcccag ctggccacct agaagtctcc ctgcacagcc tctagtgtcc ggggaccttg
tgggaccagt cccacaccgc tggtccctgc cctcccctgc tcccaggttg aggtgcgctc
acctcagagc agggccaaag cacagctggg catgccatgt ctgagcggcg cagagccctc
caggcctgca ggggcaaggg gctggctgga gtctcagagc acagaggtag gagaactggg
gttcaagccc aggcttcctg ggtcctgcct ggtcctccct cccaaggagc cattctgtgt
gtgactctgg gtggaagtgc ccagcccctg cccctacggg cgctgcagcc tcccttccat
gccccaggat cactctctgc tggcaggatt cttcccgctc cccacctacc cagctgatgg
gggttggggt gcttcctttc aggccaaggc tatgaaggga cagctgctgg gacccacctc
cccctccccg gccacatgcc gcgtccctgc cccgacccgg gtctggtgct gaggatacag
ctcttctcag tgtctgaaca atctccaaaa ttgaaatgta tatttttgct aggagcccca
gcttcctgtg tttttaatat aaatagtgta cacagactga cgaaacttta aataaatggg
aattaaatat ttaa
PPARD Accession No. NP_006229 (SEQ ID NO:_{——})
MEQPQEEAPE VREEEEKEEV AEAEGAPELN GGPQHALPSS SYTDLSRSSS PPSLLDQLQM
GCDGASCGSL NMECRVCGDK ASGFHYGVHA CEGCKGFFRR TIRMKLEYEK CERSCKIQKK
NRNKCQYCRF QKCLALGMSH NAIRFGRMPE AEKRKLVAGL TANEGSQYNP QVADLKAFSK
HIYNAYLKNF NMTKKKARSI LTGKASHTAP FVIHDIETLW QAEKGLVWKQ LVNGLPPYKE
ISVHVFYRCQ CTTVETVREL TEFAKSIPSF SSLFLNDQVT LLKYGVHEAI FAMLASIVNK
DGLLVANGSG FVTREFLRSL RKPFSDIIEP KFEFAVKFNA LELDDSDLAL FIAAIILCGD
RPGLMNVPRV EAIQDTILRA LEFHLQANHP DAQYLFPKLL QKMADLRQLV TEHAQMMQRI
KKTETETSLH PLLQEIYKDM Y

What is claimed is:

1. A compound having the chemical structure 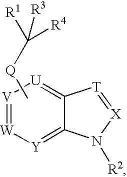
all salts, prodrugs, tautomers and isomers thereof,
wherein:
T, W, X, and Y are selected from N or CR⁵, one of U and V is C bound to Q and the other is either N or CR⁵, wherein at most two of W, Y and U or V is N and at most one of T and X is N;
Q is —O—, —S—, —NR⁶—, or —CR⁷R⁸—;
R¹ is selected from the group consisting of —C(O)OR¹⁷, —C(O)NR¹⁸R¹⁹, and a carboxylic acid isostere;
R² is selected from the group consisting of optionally substituted lower alkenyl, optionally substituted lower alkynyl, optionally substituted cycloalkyl, optionally substituted heterocycloalkyl, optionally substituted aryl, optionally substituted heteroaryl, —C(Z)NR⁹R¹⁰, —C(Z)R¹¹, —S(O)₂NR⁹R¹⁰, and —S(O)₂R²;
R³, R⁴, R⁷ and R⁸ are independently selected from the group consisting of hydrogen, optionally substituted lower alkyl, optionally substituted cycloalkyl, optionally substituted heterocycloalkyl, optionally substituted aryl, and optionally substituted heteroaryl; or
any two of R³, R⁴, R⁷ and R⁸ may combine to form optionally substituted 3-7 membered monocyclic-cycloalkyl or optionally substituted 3-7 membered monocyclic-heterocycloalkyl;
R⁵ at each occurrence is independently selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OR¹³, —SR¹⁴, —NR¹⁵R¹⁶, —C(Z)NR⁹R¹⁰, —C(Z)R¹¹, —S(O)₂NR⁹R¹⁰, and —S(O)ₙR¹²;
R⁶ is selected from the group consisting of hydrogen, optionally substituted lower alkyl, optionally substituted lower alkenyl, provided, however, that when R⁶ is optionally substituted lower alkenyl, no alkene carbon thereof is bound to the N of NR⁶, optionally substituted lower alkynyl, provided, however, that when R⁶ is optionally substituted lower alkynyl, no alkyne carbon thereof is bound to the N of NR⁶, optionally substituted cycloalkyl, optionally substituted heterocycloalkyl, optionally substituted aryl, optionally substituted heteroaryl, —C(Z)NR⁹R¹⁰, —C(Z)R¹⁰, —S(O)₂NR⁹R¹⁰, and —S(O)₂R¹²;
R⁹ and R¹⁰ at each occurrence are independently selected from the group consisting of hydrogen, optionally substituted lower alkyl, optionally substituted lower alkenyl, provided, however, that when R⁹ and/or R¹⁰ are optionally substituted lower alkenyl, no alkene carbon thereof is bound to the N of NR⁹R¹⁰, optionally substituted lower alkynyl, provided, however, that when R⁹ and/or R¹⁰ are optionally substituted lower alkynyl, no alkyne carbon thereof is bound to the N of NR⁹R¹⁰, optionally substituted cycloalkyl, optionally substituted heterocycloalkyl, optionally substituted aryl, and optionally substituted heteroaryl;
R¹¹ at each occurrence is independently selected from the group consisting of optionally substituted lower alkyl, optionally substituted lower alkenyl, provided, however, that when R¹¹ is optionally substituted lower alkenyl, no alkene carbon thereof is bound to the C(Z) of C(Z)R¹¹, optionally substituted lower alkynyl, provided, however, that when R¹¹ is optionally substituted lower alkynyl, no alkyne carbon thereof is bound to the C(Z) of C(Z)R¹¹, optionally substituted cycloalkyl, optionally substituted heterocycloalkyl, optionally substituted aryl, optionally substituted heteroaryl, and —OR¹⁴;
R¹² at each occurrence is independently selected from the group consisting of optionally substituted lower alkyl, optionally substituted lower alkenyl, provided, however, that when R¹² is optionally substituted lower alkenyl, no alkene carbon thereof is bound to the S(O)ₙ of S(O)ₙR¹², optionally substituted lower alkynyl, provided, however, that when R¹² is optionally substituted lower alkynyl, no alkyne carbon thereof is bound to the S(O)ₙ of S(O)NR¹², optionally substituted cycloalkyl, optionally substituted heterocycloalkyl, optionally substituted aryl, and optionally substituted heteroaryl;
R¹³ at each occurrence is independently selected from the group consisting of hydrogen, optionally substituted lower alkyl, optionally substituted lower alkenyl, provided, however, that when R¹³ is optionally substituted lower alkenyl, no alkene carbon thereof is bound to the O of OR¹³, optionally substituted lower alkynyl, provided, however, that when R¹³ is optionally substituted lower alkynyl, no alkyne carbon thereof is bound to the O of OR¹³, optionally substituted cycloalkyl, optionally substituted heterocycloalkyl, optionally substituted aryl, optionally substituted heteroaryl, —C(Z)R¹¹ and —C(Z)NR⁹R¹⁰;
R¹⁴ at each occurrence is independently selected from the group consisting of hydrogen, optionally substituted lower alkyl, optionally substituted lower alkenyl, provided, however, that when R¹⁴ is optionally substituted lower alkenyl, no alkene carbon thereof is bound to the S of SR¹⁴ or the O of OR¹⁴, optionally substituted lower alkynyl, provided, however, that when R¹⁴ is optionally substituted lower alkynyl, no alkyne carbon thereof is bound to the S of SR¹⁴ or the O of OR¹⁴, optionally substituted cycloalkyl, optionally substituted heterocycloalkyl, optionally substituted aryl, and optionally substituted heteroaryl;
R¹⁵ and R¹⁶ at each occurrence are independently selected from the group consisting of hydrogen, optionally substituted lower alkyl, optionally substituted lower alkenyl, provided, however, that when R¹⁵ and/or R¹⁶ are optionally substituted lower alkenyl, no alkene carbon thereof is bound to the N of NR¹⁵R¹⁶, optionally substituted lower alkynyl, provided, however, that when R¹⁵ and/or R¹⁶ are optionally substituted lower alkynyl, no alkyne carbon thereof is bound to the N of NR¹⁵R¹⁶, optionally substituted cycloalkyl, optionally substituted heterocycloalkyl, optionally substituted aryl, optionally substituted heteroaryl, —C(Z)R¹¹, —C(Z)NR⁹R¹⁰, —S(O)₂R¹², and —S(O)₂NR⁹R¹⁰;
R¹⁷ is selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁷ is lower alkyl, any substitution on the alkyl carbon bound to the O of OR¹⁷ is fluoro;
R¹⁸ and R¹⁹ are independently selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁸ and/or R¹⁹ is lower alkyl, any substitution on the alkyl carbon bound to the N of NR¹⁸R¹⁹ is fluoro; or
R¹⁸ and R¹⁹ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
n is 1 or 2; and
Z is O or S, provided, however, that when R² is —C(O)R¹¹, T and X are either N or CH, and provided the compound is not 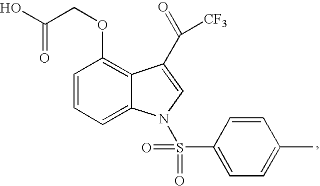
or an ester thereof.

2. The compound of claim 1, wherein T, U, W, X and Y are CR⁵ and Q is bound to V.

3. The compound of claim 2, wherein one of T, U, W, X and Y is CR⁵ and the others of T, U, W, X and Y are CH.

4. The compound of claim 2, wherein T, U, W, X and Y are CH.

5. The compound of claim 1, wherein T, V, W, X and Y are CR⁵ and Q is bound to U.

6. The compound of claim 5, wherein one of T, V, W, X and Y is CR⁵ and the others of T, V, W, X and Y are CH.

7. The compound of claim 5, wherein T, V, W, X and Y are CH.

8. The compound of any of claims 1-7, wherein R² is —S(O)₂R¹².

9. A composition comprising:
a pharmaceutically acceptable carrier; and
a compound according to claim 1.

10. A compound having the chemical structure 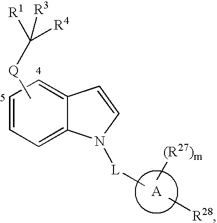
all salts, prodrugs, tautomers and isomers thereof,
wherein:
Q is —O—, —S—, —NR⁶—, or —CR⁷R⁸—, and wherein Q is bound to the carbon atom at either the 4 or the 5 position of indole with the other carbon atom at the 4 or the 5 position being substituted with hydrogen;
R¹ is selected from the group consisting of —C(O)OR¹⁷, —C(O)NR¹⁸R¹⁹, and a carboxylic acid isostere;
R³, R⁴, R⁷ and R⁸ are independently selected from the group consisting of hydrogen, optionally substituted lower alkyl, optionally substituted cycloalkyl, optionally substituted heterocycloalkyl, optionally substituted aryl, and optionally substituted heteroaryl; or
any two of R³, R⁴, R⁷ and R⁸ may combine to form optionally substituted 3-7 membered monocyclic-cycloalkyl or optionally substituted 3-7 membered monocyclic-heterocycloalkyl;
A is a monocyclic or bicyclic ring selected from the group consisting of cycloalkyl, heterocycloalkyl, aryl, and heteroaryl;
L is selected from the group consisting of —C(Z)NR²⁹—, —C(Z)-, —S(O)₂NR²⁹—, and —S(O)₂—, attached to A at any available atom to produce a stable compound;
R²⁷ at each occurrence is independently selected from the group consisting of halogen, —OH, lower alkyl, lower alkoxy, and lower alkylthio, wherein lower alkyl and the lower alkyl chains of lower alkoxy and lower alkylthio are optionally substituted with fluoro, —OH, lower alkoxy, or lower alkylthio, provided that any substitution of the carbon bound to the 0 of lower alkoxy or S of lower alkylthio is fluoro;
R²⁸ is selected from the group consisting of hydrogen, halogen, cyano, nitro, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —OR¹³, —SR¹⁴, —NR¹⁵R¹⁶, —C(Z)NR⁹R¹⁰, —C(Z)R¹¹, —S(O)₂NR⁹R¹⁰, and —S(O)ₙR¹², attached to A at any available atom to produce a stable compound;
R⁶ is selected from the group consisting of hydrogen, optionally substituted lower alkyl, optionally substituted lower alkenyl, provided, however, that when R⁶ is optionally substituted lower alkenyl, no alkene carbon thereof is bound to the N of NR⁶, optionally substituted lower alkynyl, provided, however, that when R⁶ is optionally substituted lower alkynyl, no alkyne carbon thereof is bound to the N of NR⁶, optionally substituted cycloalkyl, optionally substituted heterocycloalkyl, optionally substituted aryl, optionally substituted heteroaryl, —C(Z)NR⁹R¹⁰, —C(Z)R¹¹, —S(O)₂NR⁹R¹⁰, and —S(O)₂R¹²;
R⁹ and R¹⁰ at each occurrence are independently selected from the group consisting of hydrogen, optionally substituted lower alkyl, optionally substituted lower alkenyl, provided, however, that when R⁹ and/or R¹⁰ are optionally substituted lower alkenyl, no alkene carbon thereof is bound to the N of NR⁹R¹⁰, optionally substituted lower alkynyl, provided, however, that when R⁹ and/or R¹⁰ are optionally substituted lower alkynyl, no alkyne carbon thereof is bound to the N of NR⁹R¹⁰, optionally substituted cycloalkyl, optionally substituted heterocycloalkyl, optionally substituted aryl, and optionally substituted heteroaryl;
R¹¹ at each occurrence is independently selected from the group consisting of optionally substituted lower alkyl, optionally substituted lower alkenyl, provided, however, that when R¹¹ is optionally substituted lower alkenyl, no alkene carbon thereof is bound to the C(Z) of C(Z)R¹¹, optionally substituted lower alkynyl, provided, however, that when R¹¹ is optionally substituted lower alkynyl, no alkyne carbon thereof is bound to the C(Z) of C(Z)R¹¹, optionally substituted cycloalkyl, optionally substituted heterocycloalkyl, optionally substituted aryl, optionally substituted heteroaryl, and —OR¹⁴;
R¹² at each occurrence is independently selected from the group consisting of optionally substituted lower alkyl, optionally substituted lower alkenyl, provided, however, that when R¹² is optionally substituted lower alkenyl, no alkene carbon thereof is bound to the S(O)ₙ of S(O)ₙR¹², optionally substituted lower alkynyl, provided, however, that when R¹² is optionally substituted lower alkynyl, no alkyne carbon thereof is bound to the S(O)ₙ of S(O)ₙR¹², optionally substituted cycloalkyl, optionally substituted heterocycloalkyl, optionally substituted aryl, and optionally substituted heteroaryl;
R¹³ at each occurrence is independently selected from the group consisting of hydrogen, optionally substituted lower alkyl, optionally substituted lower alkenyl, provided, however, that when R¹³ is optionally substituted lower alkenyl, no alkene carbon thereof is bound to the O of OR¹³, optionally substituted lower alkynyl, provided, however, that when R¹³ is optionally substituted lower alkynyl, no alkyne carbon thereof is bound to the O of OR¹³, optionally substituted cycloalkyl, optionally substituted heterocycloalkyl, optionally substituted aryl, optionally substituted heteroaryl, —C(Z)R¹¹ and —C(Z)NR⁹R¹⁰;
R¹⁴ at each occurrence is independently selected from the group consisting of hydrogen, optionally substituted lower alkyl, optionally substituted lower alkenyl, provided, however, that when R¹⁴ is optionally substituted lower alkenyl, no alkene carbon thereof is bound to the S of SR¹⁴ or the O of OR¹⁴, optionally substituted lower alkynyl, provided, however, that when R¹⁴ is optionally substituted lower alkynyl, no alkyne carbon thereof is bound to the S of SR¹⁴ or the O of OR¹⁴, optionally substituted cycloalkyl, optionally substituted heterocycloalkyl, optionally substituted aryl, and optionally substituted heteroaryl;
R¹⁵ and R¹⁶ at each occurrence are independently selected from the group consisting of hydrogen, optionally substituted lower alkyl, optionally substituted lower alkenyl, provided, however, that when R¹⁵ and/or R¹⁶ are optionally substituted lower alkenyl, no alkene carbon thereof is bound to the N of NR¹⁵R¹⁶, optionally substituted lower alkynyl, provided, however, that when R¹⁵ and/or R¹⁶ are optionally substituted lower alkynyl, no alkyne carbon thereof is bound to the N of NR¹⁵R¹⁶, optionally substituted cycloalkyl, optionally substituted heterocycloalkyl, optionally substituted aryl, optionally substituted heteroaryl, —C(Z)R¹¹, —C(Z)NR⁹R¹⁰, —S(O)₂R¹², and —S(O)₂NR⁹R¹⁰;
R¹⁷ is selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁷ is lower alkyl, any substitution on the alkyl carbon bound to the O of OR¹⁷ is fluoro;
R¹⁸ and R¹⁹ are independently selected from the group consisting of hydrogen, lower alkyl, phenyl, 5-7 membered monocyclic heteroaryl, 3-7 membered monocyclic cycloalkyl, and 5-7 membered monocylic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁸ and/or R¹⁹ is lower alkyl, any substitution on the alkyl carbon bound to the N of NR¹⁸R¹⁹ is fluoro; or
R¹⁸ and R¹⁹ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R²⁹ is selected from the group consisting of hydrogen, optionally substituted lower alkyl, optionally substituted lower alkenyl, provided, however, that when R²⁹ is optionally substituted lower alkenyl, no alkene carbon thereof is bound to the N of NR²⁹, optionally substituted lower alkynyl, provided, however, that when R²⁹ is optionally substituted lower alkynyl, no alkyne carbon thereof is bound to the N of NR²⁹, optionally substituted cycloalkyl, optionally substituted heterocycloalkyl, optionally substituted aryl, and optionally substituted heteroaryl;
n is 1 or 2;
m is 0, 1, or 2; and
Z is O or S.

11. The compound of claim 10, wherein A is monocyclic aryl or monocyclic heteroaryl.

12. The compound of claim 11,
wherein:
R²⁸ is selected from the group consisting of optionally substituted cycloalkyl, optionally substituted heterocycloalkyl, optionally substituted aryl, optionally substituted heteroaryl, —OR¹³, —SR¹⁴, —NR¹⁵R¹⁶, —C(Z)NR⁹R¹⁰, —C(Z)R¹¹, —S(O)₂NR⁹R¹⁰, and —S(O)ₙR¹².

13. The compound of claim 12, wherein one of R⁹ and R¹⁰, one of R¹⁵ and R¹⁶, and R¹¹, R¹², R¹³ and R¹⁴ are selected from the group consisting of optionally substituted cycloalkyl, optionally substituted heterocycloalkyl, optionally substituted aryl, and optionally substituted heteroaryl.

14. The compound of claim 13, wherein R³ and R⁴ are H, and Q is —O—.

15. The compound of claim 13, wherein R³ and R⁴ are H, and Q is —CH₂—.

16. A composition comprising:
a pharmaceutically acceptable carrier; and
a compound according to claim 10.

17. A method for treating a subject suffering from or at risk of a disease or condition for which PPAR modulation provides a therapeutic benefit, comprising:
administering to said subject an effective amount of a PPAR modulator having the chemical structure according to claim 1.

18. A method for treating a subject suffering from or at risk of a disease or condition for which PPAR modulation provides a therapeutic benefit, comprising administering to said subject an effective amount of a PPAR modulator having the chemical structure according to claim 10.

19. The method of claim 17 or 18, wherein said compound is approved for administration to a human.

20. The method of claim 17 or 18, wherein said disease or condition is a PPAR-mediated disease or condition.

21. The method of claim 17 or 18, wherein said disease or condition is selected from the group consisting of obesity, overweight condition, bulimia, anorexia nervosa, hyperlipidemia, dyslipidemia, hypoalphalipoproteinemia, hypertriglyceridemia, hypercholesterolemia, low HDL, Metabolic Syndrome, Type II diabetes mellitus, Type I diabetes, hyperinsulinemia, impaired glucose tolerance, insulin resistance, a diabetic complication of neuropathy, nephropathy, retinopathy, diabetic foot ulcer or cataracts, hypertension, coronary heart disease, heart failure, congestive heart failure, atherosclerosis, arteriosclerosis, stroke, cerebrovascular disease, myocardial infarction, peripheral vascular disease, vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, hepatitis, eczema, psoriasis, dermatitis, impaired wound healing, Alzheimer's disease, Parkinson's disease, amyotrophic lateral sclerosis, spinal cord injury, acute disseminated encephalomyelitis, Guillain-Barre syndrome, thrombosis, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic or inflammatory pain, infertility, and cancer.

22. A kit comprising a compound according to claim 1.

23. A kit comprising a compound according to claim 10.

24. A kit comprising a composition according to claim 9.

25. A kit comprising a composition according to claim 16.
